SEQUENCE LISTING (97 sequences)

SEQ ID NO: 1 - DNA, 3180 bp, Homo sapiens
atggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggccaaccaagccctcgagcagttattcccatgtcctgtataaccaatggaagtggtgcaaacagaaaaccaagtcataccagtgctgtctcaattgcaggaaaagaaactctttcatctgctgctaaaagtggtacagaaaaaaagaaagaaaaaccacaaggacagagagaaaaaaaagaggaatctcattctaatgatcaaagtccacaaattcgagcatcaccttctccccagccctcttcacaacctctccaaatacacagacaaactccagaaagcaagaatgctactcccaccaaaagcataaaacgaccatcaccagctgaaaagtcacataattcttgggaaaattcagatgatagccgtaataaattgtcgaaaataccttcaacacccaaattaataccaaaagttaccaaaactgcagacaagcataaagatgtcatcatcaaccaagaaggagaatatattaaaatgtttatgcgcggtcggccaattaccatgttcattccttccgatgttgacaactatgatgacatcagaacggaactgcctcctgagaagctcaaactggagtgggcatatggttatcgaggaaaggactgtagagctaatgtttaccttcttccgaccggggaaatagtttatttcattgcatcagtagtagtactatttaattatgaggagagaactcagcgacactacctgggccatacagactgtgtgaaatgccttgctatacatcctgacaaaattaggattgcaactggacagatagctggcgtggataaagatggaaggcctctacaaccccacgtcagagtgtgggattctgttactctatccacactgcagattattggacttggcacttttgagcgtggagtaggatgcctggatttttcaaaagcagattcaggtgttcatttatgtgttattgatgactccaatgagcatatgcttactgtatgggactggcagaagaaagcaaaaggagcagaaataaagacaacaaatgaagttgttttggctgtggagtttcacccaacagatgcaaataccataattacatgcggtaaatctcatattttcttctggacctggagcggcaattcactaacaagaaaacagggaatttttgggaaatatgaaaagccaaaatttgtgcagtgtttagcattcttggggaatggagatgttcttactggagactcaggtggagtcatgcttatatggagcaaaactactgtagagcccacacctgggaaaggacctaaagtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctga

SEQ ID NO: 2 - DNA, 3933 bp, Homo sapiens
atggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggccaaccaagccctcgagcagttattcccatgtcctgtataaccaatggaagtggtgcaaacagaaaaccaagtcataccagtgctgtctcaattgcaggaaaagaaactctttcatctgctgctaaaagtggtacagaaaaaaagaaagaaaaaccacaaggacagagagaaaaaaaagaggaatctcattctaatgatcaaagtccacaaattcgagcatcaccttctccccagccctcttcacaacctctccaaatacacagacaaactccagaaagcaagaatgctactcccaccaaaagcataaaacgaccatcaccagctgaaaagtcacataattcttgggaaaattcagatgatagccgtaataaattgtcgaaaataccttcaacacccaaattaataccaaaagttaccaaaactgcagacaagcataaagatgtcatcatcaaccaagaaggagaatatattaaaatgtttatgcgcggtcggccaattaccatgttcattccttccgatgttgacaactatgatgacatcagaacggaactgcctcctgagaagctcaaactggagtgggcatatggttatcgaggaaaggactgtagagctaatgtttaccttcttccgaccggggaaatagtttatttcattgcatcagtagtagtactatttaattatgaggagagaactcagcgacactacctgggccatacagactgtgtgaaatgccttgctatacatcctgacaaaattaggattgcaactggacagatagctggcgtggataaagatggaaggcctctacaaccccacgtcagagtgtgggattctgttactctatccacactgcagattattggacttggcacttttgagcgtggagtaggatgcctggatttttcaaaagcagattcaggtgttcatttatgtgttattgatgactccaatgagcatatgcttactgtatgggactggcagaagaaagcaaaaggagcagaaataaagacaacaaatgaagttgttttggctgtggagtttcacccaacagatgcaaataccataattacatgcggtaaatctcatattttcttctggacctggagcggcaattcactaacaagaaaacagggaatttttgggaaatatgaaaagccaaaatttgtgcagtgtttagcattcttggggaatggagatgttcttactggagactcaggtggagtcatgcttatatggagcaaaactactgtagagcccacacctgggaaaggacctaaaggtgtatatcaaatcagcaaacaaatcaaagctcatgatggcagtgtgttcacactttgtcagatgagaaatgggatgttattaactggaggagggaaagacagaaaaataattctgtgggatcatgatctgaatcctgaaagagaaatagaggttcctgatcagtatggcacaatcagagctgtagcagaaggaaaggcagatcaatttttagtaggcacatcacgaaactttattttacgaggaacatttaatgatggcttccaaatagaagtacagggtcatacagatgagctttggggtcttgccacacatcccttcaaagatttgctcttgacatgtgctcaggacaggcaggtgtgcctgtggaactcaatggaacacaggctggaatggaccaggctggtagatgaaccaggacactgtgcagattttcatccaagtggcacagtggtggccataggaacgcactcaggcaggtggtttgttctggatgcagaaaccagagatctagtttctatccacacagacgggaatgaacagctctctgtgatgcgctactcaatagatggtaccttcctggctgtaggatctcatgacaactttatttacctctatgtagtctctgaaaatggaagaaaatatagcagatatggaaggtgcactggacattccagctacatcacacaccttgactggtccccagacaacaagtatataatgtctaactcgggagactatgaaatattgtacttgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctga

SEQ ID NO: 3 - DNA, 2358 bp, Homo sapiens
atggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggccaaccaagccctcgagcagttattcccatgtcctgtataaccaatggaagtggtgcaaacagaaaaccaagtcataccagtgctgtctcaattgcaggaaaagaaactctttcatctgctgctaaaagtggtacagaaaaaaagaaagaaaaaccacaaggacagagagaaaaaaaagaggaatctcattctaatgatcaaagtccacaaattcgagcatcaccttctccccagccctcttcacaacctctccaaatacacagacaaactccagaaagcaagaatgctactcccaccaaaagcataaaacgaccatcaccagctgaaaagtcacataattcttgggaaaattcagatgatagccgtaataaattgtcgaaaataccttcaacacccaaattaataccaaaagttaccaaaactgcagacaagcataaagatgtcatcatcaaccaagtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctga

SEQ ID NO: 4 - DNA, 2391 bp, Homo sapiens
atggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggccaaccaagccctcgagcagttattcccatgtcctgtataaccaatggaagtggtgcaaacagaaaaccaagtcataccagtgctgtctcaattgcaggaaaagaaactctttcatctgctgctaaaagtggtacagaaaaaaagaaagaaaaaccacaaggacagagagaaaaaaaagaggaatctcattctaatgatcaaagtccacaaattcgagcatcaccttctccccagccctcttcacaacctctccaaatacacagacaaactccagaaagcaagaatgctactcccaccaaaagcataaaacgaccatcaccagctgaaaagtcacataattcttgggaaaattcagatgatagccgtaataaattgtcgaaaataccttcaacacccaaattaataccaaaagttaccaaaactgcagacaagcataaagatgtcatcatcaaccaagcaaaaatgtcaactcgcgaaaaaaacagccaagtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctga

SEQ ID NO: 5 - DNA, 3294 bp, Homo sapiens
atggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggccaaccaagccctcgagcagttattcccatgtcctgtataaccaatggaagtggtgcaaacagaaaaccaagtcataccagtgctgtctcaattgcaggaaaagaaactctttcatctgctgctaaaagtggtacagaaaaaaagaaagaaaaaccacaaggacagagagaaaaaaaagaggaatctcattctaatgatcaaagtccacaaattcgagcatcaccttctccccagccctcttcacaacctctccaaatacacagacaaactccagaaagcaagaatgctactcccaccaaaagcataaaacgaccatcaccagctgaaaagtcacataattcttgggaaaattcagatgatagccgtaataaattgtcgaaaataccttcaacacccaaattaataccaaaagttaccaaaactgcagacaagcataaagatgtcatcatcaaccaagaaggagaatatattaaaatgtttatgcgcggtcggccaattaccatgttcattccttccgatgttgacaactatgatgacatcagaacggaactgcctcctgagaagctcaaactggagtgggcatatggttatcgaggaaaggactgtagagctaatgtttaccttcttccgaccggggaaatagtttatttcattgcatcagtagtagtactatttaattatgaggagagaactcagcgacactacctgggccatacagactgtgtgaaatgccttgctatacatcctgacaaaattaggattgcaactggacagatagctggcgtggataaagatggaaggcctctacaaccccacgtcagagtgtgggattctgttactctatccacactgcagattattggacttggcacttttgagcgtggagtaggatgcctggatttttcaaaagcagattcaggtgttcatttatgtgttattgatgactccaatgagcatatgcttactgtatgggactggcagaagaaagcaaaaggagcagaaataaagacaacaaatgaagttgttttggctgtggagtttcacccaacagatgcaaataccataattacatgcggtaaatctcatattttcttctggacctggagcggcaattcactaacaagaaaacagggaatttttgggaaatatgaaaagccaaaatttgtgcagtgtttagcattcttggggaatggagatgttcttactggagactcaggtggagtcatgcttatatggagcaaaactactgtagagcccacacctgggaaaggacctaaaggtgtatatcaaatcagcaaacaaatcaaagctcatgatggcagtgtgttcacactttgtcagatgagaaatgggatgttattaactggaggagggaaagacagaaaaataattctgtgggatcatgatctgaatcctgaaagagaaatagagatatgctggatgagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctga

SEQ ID NO: 6 - DNA, 1899 bp, Homo sapiens
atggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaagtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctga

SEQ ID NO: 7 - DNA, 2016 bp, Homo sapiens
atggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggttcagagctcaggggaggatatggagatccagggaggcttcctgtaggaagtggcctgtgtagtgcttcaagggccaggctgccaggccatgttgcagctgaccacccacctgcagtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctga

SEQ ID NO: 8 - DNA, 3747 bp, Homo sapiens
atggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggccaaccaagccctcgagcagttattcccatgtcctgtataaccaatggaagtggtgcaaacagaaaaccaagtcataccagtgctgtctcaattgcaggaaaagaaactctttcatctgctgctaaaagtggtacagaaaaaaagaaagaaaaaccacaaggacagagagaaaaaaaagaggaatctcattctaatgatcaaagtccacaaattcgagcatcaccttctccccagccctcttcacaacctctccaaatacacagacaaactccagaaagcaagaatgctactcccaccaaaagcataaaacgaccatcaccagctgaaaagtcacataattcttgggaaaattcagatgatagccgtaataaattgtcgaaaataccttcaacacccaaattaataccaaaagttaccaaaactgcagacaagcataaagatgtcatcatcaaccaagaaggagaatatattaaaatgtttatgcgcggtcggccaattaccatgttcattccttccgatgttgacaactatgatgacatcagaacggaactgcctcctgagaagctcaaactggagtgggcatatggttatcgaggaaaggactgtagagctaatgtttaccttcttccgaccggggaaatagtttatttcattgcatcagtagtagtactatttaattatgaggagagaactcagcgacactacctgggccatacagactgtgtgaaatgccttgctatacatcctgacaaaattaggattgcaactggacagatagctggcgtggataaagatggaaggcctctacaaccccacgtcagagtgtgggattctgttactctatccacactgcagattattggacttggcacttttgagcgtggagtaggatgcctggatttttcaaaagcagattcaggtgttcatttatgtgttattgatgactccaatgagcatatgcttactgtatgggactggcagaagaaagcaaaaggagcagaaataaagacaacaaatgaagttgttttggctgtggagtttcacccaacagatgcaaataccataattacatgcggtaaatctcatattttcttctggacctggagcggcaattcactaacaagaaaacagggaatttttgggaaatatgaaaagccaaaatttgtgcagtgtttagcattcttggggaatggagatgttcttactggagactcaggtggagtcatgcttatatggagcaaaactactgtagagcccacacctgggaaaggacctaaaggtgtatatcaaatcagcaaacaaatcaaagctcatgatggcagtgtgttcacactttgtcagatgagaaatgggatgttattaactggaggagggaaagacagaaaaataattctgtgggatcatgatctgaatcctgaaagagaaatagaggttcctgatcagtatggcacaatcagagctgtagcagaaggaaaggcagatcaatttttagtaggcacatcacgaaactttattttacgaggaacatttaatgatggcttccaaatagaagtacagggtcatacagatgagctttggggtcttgccacacatcccttcaaagatttgctcttgacatgtgctcaggacaggcaggtgtgcctgtggaactcaatggaacacaggctggaatggaccaggctggtagatgaaccaggacactgtgcagattttcatccaagtggcacagtggtggccataggaacgcactcaggcaggtggtttgttctggatgcagaaaccagagatctagtttctatccacacagacgggaatgaacagctctctgtgatgcgctactcaatagtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctga

SEQ ID NO: 9 - DNA, 2614 bp, Homo sapiens
cctccgcaagccgtctttctctagagttgtatatatagaacatcctggagtccaccatgaacggacagttggatctaagtgggaagctaatcatcaaagctcaacttggggaggatattcggcgaattcctattcataatgaagatattacttatgatgaattagtgctaatgatgcaacgagttttcagaggaaaacttctgagtaatgatgaagtaacaataaagtataaagatgaagatggagatcttataacaatttttgatagttctgacctttcctttgcaattcagtgcagtaggatactgaaactgacattatttgttaatggccagccaagaccccttgaatcaagtcaggtgaaatatctccgtcgagaactgatagaacttcgaaataaagtgaatcgtttattggatagcttggaaccacctggagaaccaggaccttccaccaatattcctgaaaatgtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtggaggagagagaggcaatggctccttcacaaaccagagaccaaatgtcacgttttgttttgtgccaacctattttgaagtaccaccaaaaaagctgtattttgaaaatgctttagaaaggttttgagcatgggttcatcctattctttcgaaagaagaaaatatcataaaaatgagtgataaatacaaggcccagatgtggttgcataaggtttttatgcatgtttgttgtatacttccttatgcttcttttaaattgtgtgtgctctgcttcaatgtagtcagaattagctgcttctatgtttcatagttggggtcatagatgtttccttgccttgttgatgtggacatgagccatttgaggggagagggaacggaaataaaggagttatttgtaatgactaaaa

SEQ ID NO: 10 - DNA, 4479 bp, Homo sapiens
tgcgagaaagatggcggacctggccgagtgcaacatcaaagtgatgtgtcgcttcagacctctcaacgagtctgaagtgaaccgcggcgacaagtacatcgccaagtttcagggagaagacacggtcgtgatcgcgtccaagccttatgcatttgatcgggtgttccagtcaagcacatctcaagagcaagtgtataatgactgtgcaaagaagattgttaaagatgtacttgaaggatataatggaacaatatttgcatatggacaaacatcctctgggaagacacacacaatggagggtaaacttcatgatccagaaggcatgggaattattccaagaatagtgcaagatatttttaattatatttactccatggatgaaaatttggaatttcatattaaggtttcatattttgaaatatatttggataagataagggacctgttagatgtttcaaagaccaacctttcagttcatgaagacaaaaaccgagttccctatgtaaaggggtgcacagagcgttttgtatgtagtccagatgaagttatggataccatagatgaaggaaaatccaacagacatgtagcagttacaaatatgaatgaacatagctctaggagtcacagtatatttcttattaatgtcaaacaagagaacacacaaacggaacaaaagctgagtggaaaactttatctggttgatttagctggtagtgaaaaggttagtaaaactggagctgaaggtgctgtgctggatgaagctaaaaacatcaacaagtcactttctgctcttggaaatgttatttctgctttggctgagggtagtacatatgttccatatcgagatagtaaaatgacaagaatccttcaagattcattaggtggcaactgtagaaccactattgtaatttgctgctctccatcatcatacaatgagtctgaaacaaaatctacactcttatttggccaaagggccaaaacaattaagaacacagtttgtgtcaatgtggagttaactgcagaacagtggaaaaagaagtatgaaaaagaaaaagaaaaaaataagatcctgcggaacactattcagtggcttgaaaatgagctcaacagatggcgtaatggggagacggtgcctattgatgaacagtttgacaaagagaaagccaacttggaagctttcacagtggataaagatattactcttaccaatgataaaccagcaaccgcaattggagttataggaaattttactgatgctgaaagaagaaagtgtgaagaagaaattgctaaattatacaaacagcttgatgacaaggatgaagaaattaaccagcaaagtcaactggtagagaaactgaagacgcaaatgttggatcaggaggagcttttggcatctaccagaagggatcaagacaatatgcaagctgagctgaatcgccttcaagcagaaaatgatgcctctaaagaagaagtgaaagaagttttacaggccctagaagaacttgctgtcaattatgatcagaagtctcaggaagttgaagacaaaactaaggaatatgaattgcttagtgatgaattgaatcagaaatcggcaactttagcgagtatagatgctgagcttcagaaacttaaggaaatgaccaaccaccagaaaaaacgagcagctgagatgatggcatctttactaaaagaccttgcagaaataggaattgctgtgggaaataatgatgtaaagcagcctgagggaactggcatgatagatgaagagttcactgttgcaagactctacattagcaaaatgaagtcagaagtaaaaaccatggtgaaacgttgcaagcagttagaaagcacacaaactgagagcaacaaaaaaatggaagaaaatgaaaaggagttagcagcatgtcagcttcgtatctctcaacatgaagccaaaatcaagtcattgactgaataccttcaaaatgtggaacaaaagaaaagacagttggaggaatctgtcgatgccctcagtgaagaactagtccagcttcgagcacaagagaaagtccatgaaatggaaaaggagcacttaaataaggttcagactgcaaatgaagttaagcaagctgttgaacagcagatccagagccatagagaaactcatcaaaaacagatcagtagtttgagagatgaagtagaagcaaaagcaaaacttattactgatcttcaagaccaaaaccagaaaatgatgttagagcaggaacgtctaagagtagaacatgagaagttgaaagccacagatcaggaaaagagcagaaaactacatgaacttacggttatgcaagatagacgagaacaagcaagacaagacttgaagggtttggaagagacagtggcaaaagaacttcagactttacacaacctgcgcaaactctttgttcaggacctggctacaagagttaaaaagagtgctgagattgattctgatgacaccggaggcagcgctgctcagaagcaaaaaatctcctttcttgaaaataatcttgaacagctcactaaagtgcacaaacagttggtacgtgataatgcagatctccgctgtgaacttcctaagttggaaaagcgacttcgagctacagctgagagagtgaaagctttggaatcagcactgaaagaagctaaagaaaatgcatctcgtgatcgcaaacgctatcagcaagaagtagatcgcataaaggaagcagtcaggtcaaagaatatggccagaagagggcattctgcacagattgtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactca

SEQ ID NO: 11 - DNA, 6267 bp, Homo sapiens
agctgcaagtggcgggcgcccaggcagatgcgatccagcggctctgggggcggcagcggtggtagcagctggtacctcccgccgcctctgttcggagggtcgcggggcaccgaggtgctttccggccgccctctggtcggccacccaaagccgcgggcgctgatgatgggtgaggagggggcggcaagatttcgggcgcccctgccctgaacgccctcagctgctgccgccggggccgctccagtgcctgcgaactctgaggagccgaggcgccggtgagagcaaggacgctgcaaacttgcgcagcgcgggggctgggattcacgcccagaagttcagcaggcagacagtccgaagccttcccgcagcggagagatagcttgagggtgcgcaagacggcagcctccgccctcggttcccgcccagaccgggcagaagagcttggaggagccaaaaggaacgcaaaaggcggccaggacagcgtgcagcagctgggagccgccgttctcagccttaaaagttgcagagattggaggctgccccgagaggggacagaccccagctccgactgcggggggcaggagaggacggtacccaactgccacctcccttcaaccatagtagttcctctgtaccgagcgcagcgagctacagacgggggcgcggcactcggcgcggagagcgggaggctcaaggtcccagccagtgagcccagtgtgcttgagtgtctctggactcgcccctgagcttccaggtctgtttcatttagactcctgctcgcctccgtgcagttgggggaaagcaagagacttgcgcgcacgcacagtcctctggagatcaggtggaaggagccgctgggtaccaaggactgttcagagcctcttcccatctcggggagagcgaagggtgaggctgggcccggagagcagtgtaaacggcctcctccggcgggatgggagccatcgggctcctgtggctcctgccgctgctgctttccacggcagctgtgggctccgggatggggaccggccagcgcgcgggctccccagctgcggggccgccgctgcagccccgggagccactcagctactcgcgcctgcagaggaagagtctggcagttgacttcgtggtgccctcgctcttccgtgtctacgcccgggacctactgctgccaccatcctcctcggagctgaaggctggcaggcccgaggcccgcggctcgctagctctggactgcgccccgctgctcaggttgctggggccggcgccgggggtctcctggaccgccggttcaccagccccggcagaggcccggacgctgtccagggtgctgaagggcggctccgtgcgcaagctccggcgtgccaagcagttggtgctggagctgggcgaggaggcgatcttggagggttgcgtcgggccccccggggaggcggctgtggggctgctccagttcaatctcagcgagctgttcagttggtggattcgccaaggcgaagggcgactgaggatccgcctgatgcccgagaagaaggcgtcggaagtgggcagagagggaaggctgtccgcggcaattcgcgcctcccagccccgccttctcttccagatcttcgggactggtcatagctccttggaatcaccaacaaacatgccttctccttctcctgattattttacatggaatctcacctggataatgaaagactccttccctttcctgtctcatcgcagccgatatggtctggagtgcagctttgacttcccctgtgagctggagtattcccctccactgcatgacctcaggaaccagagctggtcctggcgccgcatcccctccgaggaggcctcccagatggacttgctggatgggcctggggcagagcgttctaaggagatgcccagaggctcctttctccttctcaacacctcagctgactccaagcacaccatcctgagtccgtggatgaggagcagcagtgagcactgcacactggccgtctcggtgcacaggcacctgcagccctctggaaggtacattgcccagctgctgccccacaacgaggctgcaagagagatcctcctgatgcccactccagggaagcatggttggacagtgctccagggaagaatcgggcgtccagacaacccatttcgagtggccctggaatacatctccagtggaaaccgcagcttgtctgcagtggacttctttgccctgaagaactgcagtgaaggaacatccccaggctccaagatggccctgcagagctccttcacttgttggaatgggacagtcctccagcttgggcaggcctgtgacttccaccaggactgtgcccagggagaagatgagagccagatgtgccggaaactgcctgtgggtttttactgcaactttgaagatggcttctgtggctggacccaaggcacactgtcaccccacactcctcaatggcaggtcaggaccctaaaggatgcccggttccaggaccaccaagaccatgctctattgctcagtaccactgatgtccccgcttctgaaagtgctacagtgaccagtgctacgtttcctgcaccgatcaagagctctccatgtgagctccgaatgtcctggctcattcgtggagtcttgaggggaaacgtgtccttggtgctagtggagaacaaaaccgggaaggagcaaggcaggatggtctggcatgtcgccgcctatgaaggcttgagcctgtggcagtggatggtgttgcctctcctcgatgtgtctgacaggttctggctgcagatggtcgcatggtggggacaaggatccagagccatcgtggcttttgacaatatctccatcagcctggactgctacctcaccattagcggagaggacaagatcctgcagaatacagcacccaaatcaagaaacctgtttgagagaaacccaaacaaggagctgaaacccggggaaaattcaccaagacagacccccatctttgaccctacagttcattggctgttcaccacatgtggggccagcgggccccatggccccacccaggcacagtgcaacaacgcctaccagaactccaacctgagcgtggaggtggggagcgagggccccctgaaaggcatccagatctggaaggtgccagccaccgacacctacagcatctcgggctacggagctgctggcgggaaaggcgggaagaacaccatgatgcggtcccacggcgtgtctgtgctgggcatcttcaacctggagaaggatgacatgctgtacatcctggttgggcagcagggagaggacgcctgccccagtacaaaccagttaatccagaaagtctgcattggagagaacaatgtgatagaagaagaaatccgtgtgaacagaagcgtgcatgagtgggcaggaggcggaggaggagggggtggagccacctacgtatttaagatgaaggatggagtgccggtgcccctgatcattgcagccggaggtggtggcagggcctacggggccaagacagacacgttccacccagagagactggagaataactcctcggttctagggctaaacggcaattccggagccgcaggtggtggaggtggctggaatgataacacttccttgctctgggccggaaaatctttgcaggagggtgccaccggaggacattcctgcccccaggccatgaagaagtgggggtgggagacaagagggggtttcggagggggtggaggggggtgctcctcaggtggaggaggcggaggatatataggcggcaatgcagcctcaaacaatgaccccgaaatggatggggaagatggggtttccttcatcagtccactgggcatcctgtacaccccagctttaaaagtgatggaaggccacggggaagtgaatattaagcattatctaaactgcagtcactgtgaggtagacgaatgtcacatggaccctgaaagccacaaggtcatctgcttctgtgaccacgggacggtgctggctgaggatggcgtctcctgcattgtgtcacccaccccggagccacacctgccactctcgctgatcctctctgtggtgacctctgccctcgtggccgccctggtcctggctttctccggcatcatgattgtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagagatctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtggaggagagagaggcaatggctccttcacaaaccagagaccaaatgtcacgttttgttttgtgccaacctattttgaagtaccaccaaaaaagctgtattttgaaaatgctttagaaaggttttgagcatgggttcatcctattctttcgaaagaagaaaatatcataaaaatgagtgataaatacaaggcccagatgtggttgcataaggtttttatgcatgtttgttgtatacttccttatgcttctttcaaattgtgtgtgctctgcttcaatgtagtcagaattagctgcttctatgtttcatagttggggtcatagatgtttccttgccttgttgatgtggacatgagccatttgaggggagagggaacggaaataaaggagttatttgtaatgactaaaa

SEQ ID NO: 12 - DNA, 5565 bp, Homo sapiens
ggcgcggcgctcgcggctgctgcctgggagggaggccgggcaggcggctgagcggcgcggctctcaacgtgacggggaagtggttcgggcggccgcggcttactaccccagggcgaacggacggacgacggaggcgggagccggtagccgagccgggcgacctagagaacgagcgggtcaggctcagcgtcggccactctgtcggtccgctgaatgaagtgcccgcccctctaagcccggagcccggcgctttccccgcaagatggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggccaaccaagccctcgagcagttattcccatgtcctgtataaccaatggaagtggtgcaaacagaaaaccaagtcataccagtgctgtctcaattgcaggaaaagaaactctttcatctgctgctaaaagtggtacagaaaaaaagaaagaaaaaccacaaggacagagagaaaaaaaagaggaatctcattctaatgatcaaagtccacaaattcgagcatcaccttctccccagccctcttcacaacctctccaaatacacagacaaactccagaaagcaagaatgctactcccaccaaaagcataaaacgaccatcaccagctgaaaagtcacataattcttgggaaaattcagatgatagccgtaataaattgtcgaaaataccttcaacacccaaattaataccaaaagttaccaaaactgcagacaagcataaagatgtcatcatcaaccaagaaggagaatatattaaaatgtttatgcgcggtcggccaattaccatgttcattccttccgatgttgacaactatgatgacatcagaacggaactgcctcctgagaagctcaaactggagtgggcatatggttatcgaggaaaggactgtagagctaatgtttaccttcttccgaccggggaaatagtttatttcattgcatcagtagtagtactatttaattatgaggagagaactcagcgacactacctgggccatacagactgtgtgaaatgccttgctatacatcctgacaaaattaggattgcaactggacagatagctggcgtggataaagatggaaggcctctacaaccccacgtcagagtgtgggattctgttactctatccacactgcagattattggacttggcacttttgagcgtggagtaggatgcctggatttttcaaaagcagattcaggtgttcatttatgtgttattgatgactccaatgagcatatgcttactgtatgggactggcagaagaaagcaaaaggagcagaaataaagacaacaaatgaagttgttttggctgtggagtttcacccaacagatgcaaataccataattacatgcggtaaatctcatattttcttctggacctggagcggcaattcactaacaagaaaacagggaatttttgggaaatatgaaaagccaaaatttgtgcagtgtttagcattcttggggaatggagatgttcttactggagactcaggtggagtcatgcttatatggagcaaaactactgtagagcccacacctgggaaaggacctaaaggtgtatatcaaatcagcaaacaaatcaaagctcatgatggcagtgtgttcacactttgtcagatgagaaatgggatgttattaactggaggagggaaagacagaaaaataattctgtgggatcatgatctgaatcctgaaagagaaatagaggttcctgatcagtatggcacaatcagagctgtagcagaaggaaaggcagatcaatttttagtaggcacatcacgaaactttattttacgaggaacatttaatgatggcttccaaatagaagtacagggtcatacagatgagctttggggtcttgccacacatcccttcaaagatttgctcttgacatgtgctcaggacaggcaggtgtgcctgtggaactcaatggaacacaggctggaatggaccaggctggtagatgaaccaggacactgtgcagattttcatccaagtggcacagtggtggccataggaacgcactcaggcaggtggtttgttctggatgcagaaaccagagatctagtttctatccacacagacgggaatgaacagctctctgtgatgcgctactcaatagatggtaccttcctggctgtaggatctcatgacaactttatttacctctatgtagtctctgaaaatggaagaaaatatagcagatatggaaggtgcactggacattccagctacatcacacaccttgactggtccccagacaacaagtatataatgtctaactcgggagactatgaaatattgtactgggacattccaaatggctgcaaactaatcaggaatcgatcggattgtaaggacattgattggacgacatatacctgtgtgctaggatttcaagtatttggtgtctggccagaaggatctgatgggacagatatcaatgcactggtgcgatcccacaatagaaaggtgatagctgttgccgatgacttttgtaaagtccatctgtttcagtatccctgctccaaagcaaaggctcccagtcacaagtacagtgcccacagcagccatgtcaccaatgtcagttttactcacaatgacagtcacctgatatcaactggtggaaaagacatgagcatcattcagtggaaacttgtggaaaagttatctttgcctcagaatgagactgtagcggatactactctaaccaaagcccccgtctcttccactgaaagtgtcatccaatctaatactcccacaccgcctccttctcagcccttaaatgagacagctgaagaggaaagtagaataagcagttctcccacacttctggagaacagcctggaacaaactgtggagccaagtgaagaccacagcgaggaggagagtgaagagggcagcggagaccttggtgagcctctttatgaagagccatgcaacgagataagcaaggagcaggccaaagccacccttctggaggaccagcaagacccttcgccctcgtcctaacaccctggcttcagtgcaactcttttccttcagctgcatgtgattttgtgataaagttcaggtaacaggatgggcagtgatggagaatcactgttgattgagattttggtttccatgtgatttgttttcttcaatagtcttattttcagtctctcaaatacagccaacttaaagttttagtttggtgtttattgaaaattaaccaaacttaatactaggagaagactgaatcattaatgatgtctcacaaattactgtgtacctaagtggtgtgatgtaaatactggaaacaaaaacagcagttgcattgattttgaaaacaaacccccttgttatctgaacatgttttcttcaggaacaaccagaggtatcacaaacactgttactcatctactggctcagactgtactacttttttttttttttttcctgaaaaagaaaccagaaaaaaatgtactcttactgagataccctctcaccccaaatgtgtaatggaaaatttttaattaagaaaaacttcagttttgccaagtgcaatggtgttgccttctttaaaaaatgccgttttcttacactaccagtggatgtccagacatgctcttagtctactagagaggtgctgccttttctaagtcataatgaggaacagtcccttaatttcttgtgtgcaactctgttttatcctagaactaagagagcattggtttgttaaagagctttcaatgtatattaaaaccttcaatactcagaaatgatggattcctccaaggagtcctttactagcctaaacattctcaaatgtttgagattcaagtgaatggaaggaaaaccacatgcctttaaaactaaactgtaataattacctggctaatttcagctaagccttcatcataatttgttccctcagtaataggagaaatataaatacagtaagtttagattattgaattggtgcttgaaatttattggttttgttgtaattttatacagattatatgagggataagatactcatcaaattgcaaattcttttttttacagaagtgtgggtaacagtcacagcagttttttttaccaacagcatacttaacagacttgctgtgtagcagtttttttctggtggagttgctgtaagtcttgtaagtctaatgtggctatcctactcttttgggcaatgcatgtattatgcattggaaaggtattttttttaagttctgttggctagctatggttttcagtacatttcctactttaagagtaattactgacaaatatgtatttcctatatgtttatactttgattataaaaaagtattttgttttgattttttaacttgctgcattgttttgatactttctatttttttggtcaaatcatgtttagaaactttggatgagttaagaagtcttaagtatgcaggcgtttacgtgattgtgccattccaaagtgcatcagaactgtcattcccttctaatatcttctcaggagtaatacaaatcaggtatttcatcatcatttggtaatatgaaaactccagtgaactcccaaggacatttacaacatttatattcacacgctgtatggaagggtgtgggtgtgtgtgaaggggcgagtggagacactgtgtgtatctctagataagaagatatgcaccacgttgaaaatactcagtgtagatctctatgtgtataggtatctgtatatctttccttttgtttacaactgttaaaaaacctcaaaatagttctcttcaaaagaagagagattccaagcaacccatctttcttcagtatgtatgttctgtacatacttatcggagcgcgccagtaagtatcaggcatatatatctgtctgttagcaatgattattacatcatcagatcagcatgtgctatactccctgcaagaaatatactgacatgaacaggcagttcttggagaagaaagagcatttctttaagtacctggggaatacagctctcagtgatcagcagggagtttatttgaggacatcagtcacctttggggttgccatgtacaatgagatttataatcatgatactcttcggtggtagtttcaaaagacactactaatacgcaggaagcgttccagctatttaatgctggcaactactgtttaatggtcagttaaatctgtgataatggttggaagtgggtggggttatgaaattgtagatgtttttagaaaaacttgtgaatgaaaatgaatccaagtgtttcatgtgaagatgttgagccattgctatcatgcattcctgtctcatggcagaaaattttgaagattaaaaaataaaataatcaaaatgtttcctctttctaaaaaaaaaaaaaaaaaa

SEQ ID NO: 13 - DNA, 2175 bp, Homo sapiens
atggctccctggcctgaattgggagatgcccagcccaaccccgataagtacctcgaaggggccgcaggtcagcagcccactgcccctgataaaagcaaagagaccaacaaaacagataacactgaggcacctgtaaccaagattgaacttctgccgtcctactccacggctacactgatagatgagcccactgaggtggatgacccctggaacctacccactcttcaggactcggggatcaagtggtcagagagagacaccaaagggaagattctctgtttcttccaagggattgggagattgattttacttctcggatttctctactttttcgtgtgctccctggatattcttagtagcgccttccagctggttggagctggagtcccaaataaaccaggcattcccaaattactagaagggagtaaaaattcaatacagtgggagaaagctgaagataatggatgtagaattacatactatatccttgagataagaaagagcacttcaaataatttacagaaccagaatttaaggtggaagatgacatttaatggatcctgcagtagtgtttgcacatggaagtccaaaaacctgaaaggaatatttcagttcagagtagtagctgcaaataatctagggtttggtgaatatagtggaatcagtgagaatattatattagttggagatgatttttggataccagaaacaagtttcatacttactattatagttggaatatttctggttgttacaatcccactgacctttgtctggcatagaagattaaagaatcaaaaaagtgccaaggaaggggtgacagtgcttataaacgaagacaaagagttggctgagctgcgaggtctggcagccggagtaggcctggctaatgcctgctatgcaatacatactcttccaacccaagaggagattgaaaatcttcctgccttccctcgggaaaaactgactctgcgtctcttgctgggaagtggagcctttggagaagtgtatgaaggaacagcagtggacatcttaggagttggaagtggagaaatcaaagtagcagtgaagactttgaagaagggttccacagaccaggagaagattgaattcctgaaggaggcacatctgatgagcaaatttaatcatcccaacattctgaagcagcttggagtttgtctgctgaatgaaccccaatacattatcctggaactgatggagggaggagaccttcttacttatttgcgtaaagcccggatggcaacgttttatggtcctttactcaccttggttgaccttgtagacctgtgtgtagatatttcaaaaggctgtgtctacttggaacggatgcatttcattcacagggatctggcagctagaaattgccttgtttccgtgaaagactataccagtccacggatagtgaagattggagactttggactcgccagagacatctataaaaatgattactatagaaagagaggggaaggcctgctcccagttcggtggatggctccagaaagtttgatggatggaatcttcactactcaatctgatgtatggtcttttggaattctgatttgggagattttaactcttggtcatcagccttatccagctcattccaaccttgatgtgttaaactatgtgcaaacaggagggagactggagccaccaagaaattgtcctgatgatctgtggaatttaatgacccagtgctgggctcaagaacccgaccaaagacctacttttcatagaattcaggaccaacttcagttattcagaaattttttcttaaatagcatttataagtccagagatgaagcaaacaacagtggagtcataaatgaaagctttgaaggtgaagatggcgatgtgatttgtttgaattcagatgacattatgccagttgctttaatggaaacgaagaaccgagaagggttaaactatatggtacttgctacagaatgtggccaaggtgaagaaaagtctgagggtcctctaggctcccaggaatctgaatcttgtggtctgaggaaagaagagaaggaaccacatgcagacaaagatttctgccaagaaaaacaagtggcttactgcccttctggcaagcctgaaggcctgaactatgcctgtctcactcacagtggatatggagatgggtctgattaa

SEQ ID NO: 14 - DNA, 1865 bp, Homo sapiens
atggctccctggcctgaattgggagatgcccagcccaaccccgataagtacctcgaaggggccgcaggtagcagcccactgcccctgataaaagcaaagagaccaacaaaacagataacactgaggcacctgtaaccaagattgaacttctgccgtcctactccacggctacactgatagatgagcccactgaggtggatgacccctggaacctacccactcttcaggactcggggatcaagtggtcagagagagacaccaaagggaagattctctgtttcttccaagggattgggagattgattttacttctcggatttctctactttttcgtgtgctccctggatattcttagtagcgccttccagctggttggagatgatttttggataccagaaacaagtttcatacttactattatagttggaatatttctggttgttacaatcccactgacctttgtctggcatagaagattaaagaatcaaaaaagtgccaaggaaggggtgacagtgcttataaacgaagacaaagagttggctgagctgcgaggtctggcagccggagtaggcctggctaatgcctgctatgcaatacatactcttccaacccaagaggagattgaaaatcttcctgccttccctcgggaaaaactgactctgcgtctcttgctgggaagtggagcctttggagaagtgtatgaaggaacagcagtggacatcttaggagttggaagtggagaaatcaaagtagcagtgaagactttgaagaagggttccacagaccaggagaagattgaattcctgaaggaggcacatctgatgagcaaatttaatcatcccaacattctgaagcagcttggagtttgtctgctgaatgaaccccaatacattatcctggaactgatggagggaggagaccttcttacttatttgcgtaaagcccggatggcaacgttttatggtcctttactcaccttggttgaccttgtagacctgtgtgtagatatttcaaaaggctgtgtctacttggaacggatgcatttcattcacagggatctggcagctagaaattgccttgtttccgtgaaagactataccagtccacggatagtgaagattggagactttggactcgccagagacatctataaaaatgattactatagaaagagaggggaaggcctgctcccagttcggtggatggctccagaaagtttgatggatggaatcttcactactcaatctgatgtatggtcttttggaattctgatttgggagattttaactcttggtcatcagccttatccagctcattccaaccttgatgtgttaaactatgtgcaaacaggagggagactggagccaccaagaaattgtcctgatgatctgtggaatttaatgacccagtgctgggctcaagaacccgaccaaagacctacttttcatagaattcaggaccaacttcagttattcagaaattttttcttaaatagcatttataagtccagagatgaagcaaacaacagtggagtcataaatgaaagctttgaaggtgaagatggcgatgtgatttgtttgaattcagatgacattatgccagttgctttaatggaaacgaagaaccgagaagggttaaactatatggtacttgctacagaatgtggccaaggtgaagaaaagtctgagggtcctctaggctcccaggaatctgaatcttgtggtctgaggaaagaagagaaggaaccacatgcagacaaagatttctgccaagaaaaacaagtggcttactgcccttctggcaagcctgaaggcctgaactatgcctgtctcactcacagtggatatggagatgggtctgattaa

SEQ ID NO: 15 - DNA, 2112 bp, Homo sapiens
atgcacaggaggagaagcaggagctgtcgggaagatcagaagccagtcatggatgaccagcgcgaccttatctccaacaatgagcaactgcccatgctgggccggcgccctggggccccggagagcaagtgcagccgcggagccctgtacacaggcttttccatcctggtgactctgctcctcgctggccaggccaccaccgcctacttcctgtaccagcagcagggccggctggacaaactgacagtcacctcccagaacctgcagctggagaacctgcgcatgaagcttcccaagcctcccaagcctgtgagcaagatgcgcatggccaccccgctgctgatgcaggcgctgcccatgggagccctgccccaggggcccatgcagaatgccaccaagtatggcaacatgacagaggaccatgtgatgcacctgctccagaatgctgaccccctgaaggtgtacccgccactgaaggggagcttcccggagaacctgagacaccttaagaacaccatggagaccatagactggaaggtctttgagagctggatgcaccattggctcctgtttgaaatgagcaggcactccttggagcaaaagcccactgacgctccaccgaaagatgatttttggataccagaaacaagtttcatacttactattatagttggaatatttctggttgttacaatcccactgacctttgtctggcatagaagattaaagaatcaaaaaagtgccaaggaaggggtgacagtgcttataaacgaagacaaagagttggctgagctgcgaggtctggcagccggagtaggcctggctaatgcctgctatgcaatacatactcttccaacccaagaggagattgaaaatcttcctgccttccctcgggaaaaactgactctgcgtctcttgctgggaagtggagcctttggagaagtgtatgaaggaacagcagtggacatcttaggagttggaagtggagaaatcaaagtagcagtgaagactttgaagaagggttccacagaccaggagaagattgaattcctgaaggaggcacatctgatgagcaaatttaatcatcccaacattctgaagcagcttggagtttgtctgctgaatgaaccccaatacattatcctggaactgatggagggaggagaccttcttacttatttgcgtaaagcccggatggcaacgttttatggtcctttactcaccttggttgaccttgtagacctgtgtgtagatatttcaaaaggctgtgtctacttggaacggatgcatttcattcacagggatctggcagctagaaattgccttgtttccgtgaaagactataccagtccacggatagtgaagattggagactttggactcgccagagacatctataaaaatgattactatagaaagagaggggaaggcctgctcccagttcggtggatggctccagaaagtttgatggatggaatcttcactactcaatctgatgtatggtcttttggaattctgatttgggagattttaactcttggtcatcagccttatccagctcattccaaccttgatgtgttaaactatgtgcaaacaggagggagactggagccaccaagaaattgtcctgatgatctgtggaatttaatgacccagtgctgggctcaagaacccgaccaaagacctacttttcatagaattcaggaccaacttcagttattcagaaattttttcttaaatagcatttataagtccagagatgaagcaaacaacagtggagtcataaatgaaagctttgaaggtgaagatggcgatgtgatttgtttgaattcagatgacattatgccagttgctttaatggaaacgaagaaccgagaagggttaaactatatggtacttgctacagaatgtggccaaggtgaagaaaagtctgagggtcctctaggctcccaggaatctgaatcttgtggtctgaggaaagaagagaaggaaccacatgcagacaaagatttctgccaagaaaaacaagtggcttactgcccttctggcaagcctgaaggcctgaactatgcctgtctcactcacagtggatatggagatgggtctgattaa

SEQ ID NO: 16 - DNA, 7368 bp, Homo sapiens
caagctttcaagcattcaaaggtctaaatgaaaaaggctaagtattatttcaaaaggcaagtatatcctaatatagcaaaacaaacaaagcaaaatccatcagctactcctccaattgaagtgatgaagcccaaataattcatatagcaaaatggagaaaattagaccggccatctaaaaatctgccattggtgaagtgatgaagaacatttactgtcttattccgaagcttgtcaattttgcaactcttggctgcctatggatttctgtggtgcagtgtacagttttaaatagctgcctaaagtcgtgtgtaactaatctgggccagcagcttgaccttggcacaccacataatctgagtgaaccgtgtatccaaggatgtcacttttggaactctgtagatcagaaaaactgtgctttaaagtgtcgggagtcgtgtgaggttggctgtagcagcgcggaaggtgcatatgaagaggaagtactggaaaatgcagacctaccaactgctccctttgcttcttccattggaagccacaatatgacattacgatggaaatctgcaaacttctctggagtaaaatacatcattcagtggaaatatgcacaacttctgggaagctggacttatactaagactgtgtccagaccgtcctatgtggtcaagcccctgcaccccttcactgagtacattttccgagtggtttggatcttcacagcgcagctgcagctctactcccctccaagtcccagttacaggactcatcctcatggagttcctgaaactgcacctttgattaggaatattgagagctcaagtcccgacactgtggaagtcagctgggatccacctcaattcccaggtggacctattttgggttataacttaaggctgatcagcaaaaatcaaaaattagatgcagggacacagagaaccagtttccagttttactccactttaccaaatactatctacaggttttctattgcagcagtaaatgaagttggtgagggtccagaagcagaatctagtattaccacttcatcttcagcagttcaacaagaggaacagtggctctttttatccagaaaaacttctctaagaaagagatctttaaaacatttagtagatgaagcacattgccttcggttggatgctatataccataatattacaggaatatctgttgatgtccaccagcaaattgtttatttctctgaaggaactctcatatgggcgaagaaggctgccaacatgtctgatgtatctgacctgagaattttttacagaggttcaggattaatttcttctatctccatagattggctttatcaaagaatgtatttcatcatggatgaactggtatgtgtctgtgatttagagaactgctcaaacatcgaggaaattactccaccctctattagtgcacctcaaaaaattgtggctgattcatacaatgggtatgtcttttacctcctgagagatggcatttatagagcagaccttcctgtaccatctggccggtgtgcagaagctgtgcgtattgtggagagttgcacgttaaaggactttgcaatcaagccacaagccaagcgaatcatttacttcaatgacactgcccaagtcttcatgtcaacatttctggatggctctgcttcccatctcatcctacctcgcatcccctttgctgatgtgaaaagttttgcttgtgaaaacaatgactttcttgtcacagatggcaaggtcattttccaacaggatgctttgtcttttaatgaattcatcgtgggatgtgacctgagtcacatagaagaatttgggtttggtaacttggtcatctttggctcatcctcccagctgcaccctctgccaggccgcccgcaggagctttcggtgctgtttggctctcaccaggctcttgttcaatggaagcctcctgcccttgccataggagccaatgtcatcctgatcagtgatattattgaactctttgaattaggcccttctgcctggcagaactggacctatgaggtgaaagtatccacccaagaccctcctgaagtcactcatattttcttgaacataagtggaaccatgctgaatgtacctgagctgcagagtgctatgaaatacaaggtttctgtgagagcaagttctccaaagaggccaggcccctggtcagagccctcagtgggtactaccctggtgccagctagtgaaccaccatttatcatggctgtgaaagaagatgggctttggagtaaaccattaaatagctttggcccaggagagttcttatcctctgatataggaaatgtgtcagacatggattggtataacaacagcctctactacagtgacacgaaaggcgacgtttttgtgtggctgctgaatgggacggatatctcagagaattatcacctacccagcattgcaggagcaggggctttagcttttgagtggctgggtcactttctctactgggctggaaagacatatgtgatacaaaggcagtctgtgttgacgggacacacagacattgttacccacgtgaagctattggtgaatgacatggtggtggattcagttggtggatatctctactggaccacactctattcagtggaaagcaccagactaaatggggaaagttcccttgtactacagacacagccttggttttctgggaaaaaggtaattgctctaactttagacctcagtgatgggctcctgtattggttggttcaagacagtcaatgtattcacctgtacacagctgttcttcggggacagagcactggggataccaccatcacagaatttgcagcctggagtacttctgaaatttcccagaatgcactgatgtactatagtggtcggctgttctggatcaatggctttaggattatcacaactcaagaaataggtcagaaaaccagtgtctctgttttggaaccagccagatttaatcagttcacaattattcagacatcccttaagcccctgccagggaacttttcctttacccctaaggttattccagattctgttcaagagtcttcatttaggattgaaggaaatgcttcaagttttcaaatcctgtggaatggtccccctgcggtagactggggtgtagttttctacagtgtagaatttagtgctcattctaagttcttggctagtgaacaacactctttacctgtatttactgtggaaggactggaaccttatgccttatttaatctttctgtcactccttatacctactggggaaagggccccaaaacatctctgtcacttcgagcacctgaaacagttccatcagcaccagagaaccccagaatatttatattaccaagtggaaaatgctgcaacaagaatgaagttgtggtggaatttaggtggaacaaacctaagcatgaaaatggggtgttaacaaaatttgaaattttctacaatatatccaatcaaagtattacaaacaaaacatgtgaagactggattgctgtcaatgtcactccctcagtgatgtcttttcaacttgaaggcatgagtcccagatgctttattgccttccaggttagggcctttacatctaaggggccaggaccatatgctgacgttgtaaagtctacaacatcagaaatcaacccatttcctcacctcataactcttcttggtaacaagatagtttttttagatatggatcaaaatcaagttgtgtggacgttttcagcagaaagagttatcagtgccgtttgctacacagctgataatgagatgggatattatgctgaaggggactcactctttcttctgcacttgcacaatcgctctagctctgagcttttccaagattcactggtttttgatatcacagttattacaattgactggatttcaaggcacctctactttgcactgaaagaatcacaaaatggaatgcaagtatttgatgttgatcttgaacacaaggtgaaatatcccagagaggtgaagattcacaataggaattcaacaataatttctttttctgtatatcctcttttaagtcgcttgtattggacagaagtttccaattttggctaccagatgttctactacagtattatcagtcacaccttgcaccgaattctgcaacccacagctacaaaccaacaaaacaaaaggaatcaatgttcttgtaatgtgactgaatttgagttaagtggagcaatggctattgatacctctaacctagagaaaccattgatatactttgccaaagcacaagagatctgggcaatggatctggaaggctgtcagtgttggagagttatcacagtacctgctatgctcgcaggaaaaacccttgttagcttaactgtggatggagatcttatatactggatcatcacagcaaaggacagcacacagatttatcaggcaaagaaaggaaatggggccatcgtttcccaggtgaaggccctaaggagtaggcatatcttggcttacagttcagttatgcagccttttccagataaagcgtttctgtctctagcttcagacactgtggaaccaactatacttaatgccactaacactagcctcacaatcagattacctctggccaagacaaacctcacatggtatggcatcaccagccctactccaacatacctggtttattatgcagaagttaatgacaggaaaaacagctctgacttgaaatatagaattctggaatttcaggacagtatagctcttattgaagatttacaaccattttcaacatacatgatacagatagctgtaaaaaattattattcagatcctttggaacatttaccaccaggaaaagagatttggggaaaaactaaaaatggagtaccagaggcagtgcagctcattaatacaactgtgcggtcagacaccagcctcattatatcttggagagaatctcacaagccaaatggacctaaagaatcagtccgttatcagttggcaatctcacacctggccctaattcctgaaactcctctaagacaaagtgaatttccaaatggaaggctcactctccttgttactagactgtctggtggaaatatttatgtgttaaaggttcttgcctgccactctgaggaaatgtggtgtacagagagtcatcctgtcactgtggaaatgtttaacacaccagagaaaccttattccttggttccagagaacactagtttgcaatttaattggaaggctccattgaatgttaacctcatcagattttgggttgagctacagaagtggaaatacaatgagttttaccatgttaaaacttcatgcagccaaggtcctgcttatgtctgtaatatcacaaatctacaaccttatacttcatataatgtcagagtagtggtggtttataagacgggagaaaatagcacctcacttccagaaagctttaagacaaaagctggagtcccaaataaaccaggcattcccaaattactagaagggagtaaaaattcaatacagtgggagaaagctgaagataatggatgtagaattacatactatatccttgagataagaaagagcacttcaaataatttacagaaccagaatttaaggtggaagatgacatttaatggatcctgcagtagtgtttgcacatggaagtccaaaaacctgaaaggaatatttcagttcagagtagtagctgcaaataatctagggtttggtgaatatagtggaatcagtgagaatattatattagttggagatgatttttggataccagaaacaagtttcatacttactattatagttggaatatttctggttgttacaatcccactgacctttgtctggcatagaagattaaagaatcaaaaaagtgccaaggaaggggtgacagtgcttataaacgaagacaaagagttggctgagctgcgaggtctggcagccggagtaggcctggctaatgcctgctatgcaatacatactcttccaacccaagaggagattgaaaatcttcctgccttccctcgggaaaaactgactctgcgtctcttgctgggaagtggagcctttggagaagtgtatgaaggaacagcagtggacatcttaggagttggaagtggagaaatcaaagtagcagtgaagactttgaagaagggttccacagaccaggagaagattgaattcctgaaggaggcacatctgatgagcaaatttaatcatcccaacattctgaagcagcttggagtttgtctgctgaatgaaccccaatacattatcctggaactgatggagggaggagaccttcttacttatttgcgtaaagcccggatggcaacgttttatggtcctttactcaccttggttgaccttgtagacctgtgtgtagatatttcaaaaggctgtgtctacttggaacggatgcatttcattcacagggatctggcagctagaaattgccttgtttccgtgaaagactataccagtccacggatagtgaagattggagactttggactcgccagagacatctataaaaatgattactatagaaagagaggggaaggcctgctcccagttcggtggatggctccagaaagtttgatggatggaatcttcactactcaatctgatgtatggtcttttggaattctgatttgggagattttaactcttggtcatcagccttatccagctcattccaaccttgatgtgttaaactatgtgcaaacaggagggagactggagccaccaagaaattgtcctgatgatctgtggaatttaatgacccagtgctgggctcaagaacccgaccaaagacctacttttcatagaattcaggaccaacttcagttattcagaaattttttcttaaatagcatttataagtccagagatgaagcaaacaacagtggagtcataaatgaaagctttgaaggtgaagatggcgatgtgatttgtttgaattcagatgacattatgccagttgctttaatggaaacgaagaaccgagaagggttaaactatatggtacttgctacagaatgtggccaaggtgaagaaaagtctgagggtcctctaggctcccaggaatctgaatcttgtggtctgaggaaagaagagaaggaaccacatgcagacaaagatttctgccaagaaaaacaagtggcttactgcccttctggcaagcctgaaggcctgaactatgcctgtctcactcacagtggatatggagatgggtctgattaatagcgttgtttgggaaatagagagttgagataaacactctcattcagtagttactgaaagaaaactctgctagaatgataaatgtcatggtggtctataactccaaataaacaatgcaacgttcc

SEQ ID NO: 17 - DNA, 50 bp, Artificial sequence
  nucleic acid probe
cttgcagctcctggtgcttccggcggtacactttacttgagactgatttt

SEQ ID NO: 18 - DNA, 50 bp, Artificial sequence
  nucleic acid probe
ctggtgcttccggcggtacactattgagtagcgcatcacagagagctgtt

SEQ ID NO: 19 - DNA, 50 bp, Artificial sequence
  nucleic acid probe
tcagggctcatccagcatatctctatttctctttcaggattcagatcatg

SEQ ID NO: 20 - DNA, 50 bp, Artificial sequence
  nucleic acid probe
cctggtgcttccggcggtacacttggttgatgatgacatctttatgcttg

SEQ ID NO: 21 - DNA, 50 bp, Artificial sequence
  nucleic acid probe
ctggtgcttccggcggtacaagtacaatatttcatagtctcccgagttag

SEQ ID NO: 22 - DNA, 50 bp, Artificial sequence
  nucleic acid probe
ttctggtatccaaaaatcatccagctgcttctgatgcttctcctcccggg

SEQ ID NO: 23 - DNA, 50 bp, Artificial sequence
  nucleic acid probe
gcacagtgatttcatcttcttgttgctgaactcgtgactcaagagctgac

SEQ ID NO: 24 - DNA, 50 bp, Artificial sequence
  nucleic acid probe
ctttaatcttctatgccagacattgctctcaatgtgcccattggcctgag

SEQ ID NO: 25 - DNA, 50 bp, Artificial sequence
  nucleic acid probe
ctggtgcttccggcggtacacattttcaggaatattggtggaaggtcctg

SEQ ID NO: 26 - DNA, 50 bp, Artificial sequence
  nucleic acid probe
ctggtgcttccggcggtacacaatctgtgcagaatgccctcttctggcca

SEQ ID NO: 27 - DNA, 50 bp, Artificial sequence
  nucleic acid probe
cctggtttatttgggactccagctccaaccagctggaaggcgctactaag

SEQ ID NO: 28 - DNA, 50 bp, Artificial sequence
  nucleic acid probe
cctggtttatttgggactccagctgccaggacctccgttctctcaaagat

SEQ ID NO: 29 - DNA, 50 bp, Artificial sequence
  nucleic acid probe
ctggtgcttccggcggtacactttaggtcctttcccaggtgtgggctcta

SEQ ID NO: 30 - DNA, 50 bp, Artificial sequence
  nucleic acid probe
ctggtttatttgggactccagccagatctccagagccagacagctcaaag

SEQ ID NO: 31 - DNA, 50 bp, Artificial sequence
  nucleic acid probe
ctttaatcttctatgccagacttctccgcctgagcctcaagagacttgag

SEQ ID NO: 32 - DNA, 50 bp, Artificial sequence
  nucleic acid probe
caactgcacggaggcgagcaggagtctaaatgaaacagacctggaagctc

SEQ ID NO: 33 - DNA, 50 bp, Artificial sequence
  nucleic acid probe
tatttccgttccctctcccctcaaatggctcatgtccacatcaacaaggc

SEQ ID NO: 34 - DNA, 50 bp, Artificial sequence
  nucleic acid probe
ttcccgagggaaggcaggaagattttcaatctcctcttgggttggaagag

SEQ ID NO: 35 - DNA, 50 bp, Artificial sequence
  nucleic acid probe
cgttgccatccgggctttacgcaaataagtaagaaggtctcctccctcca

SEQ ID NO: 36 - DNA, 50 bp, Artificial sequence
  nucleic acid probe
gaaacttgtttctggtatccaaaaatcatctttcggtggagcgtcagtgg

SEQ ID NO: 37 - DNA, 50 bp, Artificial sequence
  nucleic acid probe
gaatgcctggtttatttgggactccagcctgagcctctctgctaatggtt

SEQ ID NO: 38 - DNA, 50 bp, Artificial sequence
  nucleic acid probe
gcctccctggatctccatatcctcccctgagctctgaacctttacttgag

SEQ ID NO: 39 - DNA, 50 bp, Artificial sequence
  nucleic acid probe
agctcctggtgcttccggcggtacacttggctgtttttttcgcgagttga

SEQ ID NO: 40 - DNA, 50 bp, Artificial sequence
  nucleic acid probe
gataagggccctgccctacttcctccaaatcgaggtgcaccaaaccctcg

SEQ ID NO: 41 - DNA, 50 bp, Artificial sequence
  nucleic acid probe
gtgagagccagtgatgcagctagattgtgacccagggctcatggataagc

SEQ ID NO: 42 - DNA, 50 bp, Artificial sequence
  nucleic acid probe
gaccaggcgcccaatacgaccaaatccgttgactccgaccttcaccttcc

SEQ ID NO: 43 - DNA, 50 bp, Artificial sequence
  nucleic acid probe
ggtcccacgatgatcccacttccataaggacatatctggcggaaggcctc

SEQ ID NO: 44 - DNA, 50 bp, Artificial sequence
  nucleic acid linker
gcagcgcacgtgctcagccgtagtgaaaatcagtctcaagtaaagtgtac

SEQ ID NO: 45 - DNA, 50 bp, Artificial sequence
  nucleic acid linker
tggctgtagaacacgcgagcggttcaacagctctctgtgatgcgctactc

SEQ ID NO: 46 - DNA, 50 bp, Artificial sequence
  nucleic acid linker
ctggcagccacggacgcggaacgagcatgatctgaatcctgaaagagaaa

SEQ ID NO: 47 - DNA, 50 bp, Artificial sequence
  nucleic acid linker
cgaagagatgcataacgcggcgcgccaagcataaagatgtcatcatcaac

SEQ ID NO: 48 - DNA, 50 bp, Artificial sequence
  nucleic acid linker
ggaagagctggccgacggactgacgctaactcgggagactatgaaatatt

SEQ ID NO: 49 - DNA, 50 bp, Artificial sequence
  nucleic acid linker
ggtactagcatgtggttaactggatcccgggaggagaagcatcagaagca

SEQ ID NO: 50 - DNA, 50 bp, Artificial sequence
  nucleic acid linker
ggctatgaacctcggccaacgctaagtcagctcttgagtcacgagttcag

SEQ ID NO: 51 - DNA, 50 bp, Artificial sequence
  nucleic acid linker
agttgccgggcgttccagaccgagactcaggccaatgggcacattgagag

SEQ ID NO: 52 - DNA, 50 bp, Artificial sequence
  nucleic acid linker
gccaccgaccgaagacttacatgatcaggaccttccaccaatattcctga

SEQ ID NO: 53 - DNA, 50 bp, Artificial sequence
  nucleic acid linker
gccacgtaggcaccggaggactcagtggccagaagagggcattctgcaca

SEQ ID NO: 54 - DNA, 50 bp, Artificial sequence
  nucleic acid linker
caaggactctaccggatcatatgcgcttagtagcgccttccagctggttg

SEQ ID NO: 55 - DNA, 50 bp, Artificial sequence
  nucleic acid linker
aacacgtacggagccggccctgtcaatctttgagagaacggaggtcctgg

SEQ ID NO: 56 - DNA, 50 bp, Artificial sequence
  nucleic acid linker
aggagctccgcgagggacatggtagtagagcccacacctgggaaaggacc

SEQ ID NO: 57 - DNA, 50 bp, Artificial sequence
  nucleic acid linker
acctgataaccacagtttctcccgcctttgagctgtctggctctggagat

SEQ ID NO: 58 - DNA, 50 bp, Artificial sequence
  nucleic acid linker
gaacacataccagggcgacagtcgcctcaagtctcttgaggctcaggcgg

SEQ ID NO: 59 - DNA, 50 bp, Artificial sequence
  nucleic acid linker
gatgatttaggttgcgccgcacgaggagcttccaggtctgtttcatttag

SEQ ID NO: 60 - DNA, 50 bp, Artificial sequence
  nucleic acid linker
aaacccacatagggacgcagcggatgccttgttgatgtggacatgagcca

SEQ ID NO: 61 - DNA, 50 bp, Artificial sequence
  nucleic acid linker
ccagttgaagctatcgcgaagccgactcttccaacccaagaggagattga

SEQ ID NO: 62 - DNA, 50 bp, Artificial sequence
  nucleic acid linker
cttctttcaccacgggctggttcgatggagggaggagaccttcttactta

SEQ ID NO: 63 - DNA, 50 bp, Artificial sequence
  nucleic acid linker
acaatgtggttcggagtgccgttccccactgacgctccaccgaaagatga

SEQ ID NO: 64 - DNA, 50 bp, Artificial sequence
  nucleic acid linker
tctgatcttccaccgctcccgaaagaaccattagcagagaggctcaggct

SEQ ID NO: 65 - DNA, 50 bp, Artificial sequence
  nucleic acid linker
cagggatcaatcttcccatacgcgcctcaagtaaaggttcagagctcagg

SEQ ID NO: 66 - DNA, 50 bp, Artificial sequence
  nucleic acid linker
cagggttgctacggattgtggcagatcaactcgcgaaaaaaacagccaag

SEQ ID NO: 67 - DNA, 50 bp, Artificial sequence
  nucleic acid linker
gcggactgtggtaccatgccgaccgcgagggtttggtgcacctcgatttg

SEQ ID NO: 68 - DNA, 50 bp, Artificial sequence
  nucleic acid linker
ggacgccgtccggtcctcacgtggagcttatccatgagccctgggtcaca

SEQ ID NO: 69 - DNA, 50 bp, Artificial sequence
  nucleic acid linker
gcgctcccacaacgctcgaccggcgggaaggtgaaggtcggagtcaacgg

SEQ ID NO: 70 - DNA, 50 bp, Artificial sequence
  nucleic acid linker
cgtcagtgaggaagagcgcgatgtggaggccttccgccagatatgtcctt

SEQ ID NO: 71 - DNA, 50 bp, Artificial sequence
  nucleic acid linker
cgccggaagcaccaggagctgcaagtgctctccttcactgtttggaggtg

SEQ ID NO: 72 - DNA, 50 bp, Artificial sequence
  nucleic acid linker
aatagtgtaccgccggaagcaccagtgctctccttcactgtttggaggtg

SEQ ID NO: 73 - DNA, 50 bp, Artificial sequence
  nucleic acid linker
tagagatatgctggatgagccctgatgctctccttcactgtttggaggtg

SEQ ID NO: 74 - DNA, 50 bp, Artificial sequence
  nucleic acid linker
caagtgtaccgccggaagcaccaggtgctctccttcactgtttggaggtg

SEQ ID NO: 75 - DNA, 50 bp, Artificial sequence
  nucleic acid linker
gtacttgtaccgccggaagcaccagtgctctccttcactgtttggaggtg

SEQ ID NO: 76 - DNA, 50 bp, Artificial sequence
  nucleic acid linker
gctggatgatttttggataccagaatgctctccttcactgtttggaggtg

SEQ ID NO: 77 - DNA, 50 bp, Artificial sequence
  nucleic acid linker
caacaagaagatgaaatcactgtgctgctctccttcactgtttggaggtg

SEQ ID NO: 78 - DNA, 50 bp, Artificial sequence
  nucleic acid linker
caatgtctggcatagaagattaaagtgctctccttcactgtttggaggtg

SEQ ID NO: 79 - DNA, 50 bp, Artificial sequence
  nucleic acid linker
aaatgtgtaccgccggaagcaccagtgctctccttcactgtttggaggtg

SEQ ID NO: 80 - DNA, 50 bp, Artificial sequence
  nucleic acid linker
gattgtgtaccgccggaagcaccagtgctctccttcactgtttggaggtg

SEQ ID NO: 81 - DNA, 50 bp, Artificial sequence
  nucleic acid linker
gagctggagtcccaaataaaccaggtgctctccttcactgtttggaggtg

SEQ ID NO: 82 - DNA, 50 bp, Artificial sequence
  nucleic acid linker
cagctggagtcccaaataaaccaggtgctctccttcactgtttggaggtg

SEQ ID NO: 83 - DNA, 50 bp, Artificial sequence
  nucleic acid linker
taaagtgtaccgccggaagcaccagtgctctccttcactgtttggaggtg

SEQ ID NO: 84 - DNA, 50 bp, Artificial sequence
  nucleic acid linker
ctggctggagtcccaaataaaccagtgctctccttcactgtttggaggtg

SEQ ID NO: 85 - DNA, 50 bp, Artificial sequence
  nucleic acid linker
agaagtctggcatagaagattaaagtgctctccttcactgtttggaggtg

SEQ ID NO: 86 - DNA, 50 bp, Artificial sequence
  nucleic acid linker
actcctgctcgcctccgtgcagttgtgctctccttcactgtttggaggtg

SEQ ID NO: 87 - DNA, 50 bp, Artificial sequence
  nucleic acid linker
tttgaggggagagggaacggaaatatgctctccttcactgtttggaggtg

SEQ ID NO: 88 - DNA, 50 bp, Artificial sequence
  nucleic acid linker
aaatcttcctgccttccctcgggaatgctctccttcactgtttggaggtg

SEQ ID NO: 89 - DNA, 50 bp, Artificial sequence
  nucleic acid linker
tttgcgtaaagcccggatggcaacgtgctctccttcactgtttggaggtg

SEQ ID NO: 90 - DNA, 50 bp, Artificial sequence
  nucleic acid linker
tttttggataccagaaacaagtttctgctctccttcactgtttggaggtg

SEQ ID NO: 91 - DNA, 50 bp, Artificial sequence
  nucleic acid linker
ggagtcccaaataaaccaggcattctgctctccttcactgtttggaggtg

SEQ ID NO: 92 - DNA, 50 bp, Artificial sequence
  nucleic acid linker
ggaggatatggagatccagggaggctgctctccttcactgtttggaggtg

SEQ ID NO: 93 - DNA, 50 bp, Artificial sequence
  nucleic acid linker
tgtaccgccggaagcaccaggagcttgctctccttcactgtttggaggtg

SEQ ID NO: 94 - DNA, 50 bp, Artificial sequence
  nucleic acid linker
atttggtcgtattgggcgcctggtctgctctccttcactgtttggaggtg

SEQ ID NO: 95 - DNA, 50 bp, Artificial sequence
  nucleic acid linker
gaggaagtagggcagggcccttatctgctctccttcactgtttggaggtg

SEQ ID NO: 96 - DNA, 50 bp, Artificial sequence
  nucleic acid linker
atggaagtgggatcatcgtgggacctgctctccttcactgtttggaggtg

SEQ ID NO: 97 - DNA, 50 bp, Artificial sequence
  nucleic acid linker
atctagctgcatcactggctctcactgctctccttcactgtttggaggtg

(19) United States
(12) Patent Application Publication
(10) Pub. No.: US 2013/0288915 A1
(43) Pub. Date: Oct. 31, 2013

(54) COMPOSITIONS AND METHODS FOR ALK MOLECULAR TESTING

(71) Applicant: HTG MOLECULAR DIAGNOSTICS, INC., TUCSON, AZ (US)

(72) Inventors: BRUCE E. SELIGMANN, TUCSON, AZ (US); BJ KERNS, MADISON, WI (US); MARK SCHWARTZ, TUCSON, AZ (US); JOHN W. LUECKE, MADISON, WI (US)

(73) Assignee: HTG MOLECULAR DIAGNOSTICS, INC., TUCSON, AZ (US)

(21) Appl. No.: 13/856,390

(22) Filed: Apr. 3, 2013

Related U.S. Application Data
(60) Provisional application No. 61/639,503, filed on Apr. 27, 2012.

(51) Int. Cl. C12Q 1/68

(52) U.S. Cl. CPC C12Q 1/6886
USPC 506/9; 506/16

(57) ABSTRACT

Disclosed herein are methods of predicting response of a tumor to an ALK inhibitor and methods of determining diagnosis or prognosis of a subject with a tumor. The methods can include detecting presence of an ALK gene fusion (such as EML4-ALK, TFG-ALK, or KIF5B-ALK) in a sample from a subject. Also disclosed herein are arrays for detecting the presence of ALK and/or ROS1 gene fusions in a sample. In some embodiments, the array includes one or more oligonucleotides complementary to an ALK or ROS1 gene fusion.

### CROSS-REFERENCE TO RELATED APPLICATION

This application claims priority to U.S. Provisional Application No. 61/639,503 filed Apr. 27, 2012, herein incorporated by reference in its entirety.

### FIELD

This disclosure relates to methods, arrays, and kits for detecting expression of ALK, ROS1, or gene fusions including ALK or ROS1, or combinations thereof, and methods of predicting treatment responsiveness of a tumor and methods of determining diagnosis or prognosis of a subject with a tumor.

### BACKGROUND

Many cancers are characterized by disruptions in cellular signaling pathways that lead to aberrant control of cellular processes, or to uncontrolled growth and proliferation of cells. These disruptions are often caused by genetic changes (also called mutations) that affect the activity of particular signaling proteins. Among other known examples, tyrosine kinase genes, which encode important enzymes directly regulating cell growth, have been reported to contain oncogenic mutations.

In particular, chronic myelogenous leukemia (CML) is driven by the mutant kinase fusion protein BCR/ABL, which displays constitutive activation of the ABL kinase, whereas gastrointestinal stromal tumor (GIST) is caused by activating point mutations in the c-Kit or platelet derived growth factor receptor (PDGFR) kinases. In some cases of human malignant lymphoma and inflammatory myofibroblastic tumors, the anaplastic lymphoma kinase (ALK) gene is fused with another gene (such as echinoderm microtubule associated protein like 4; EML4) as a result of chromosomal translocation or inversion and forms a fusion type tyrosine kinase. In some examples, the fusion results in loss of control of the tyrosine kinase activity of ALK and may lead to tumor formation.

The clinical success of the small molecule kinase inhibitor imatinib mesylate in CML and GIST has established a paradigm for the targeted treatment of tumors whose growth is dependent on specific kinases. Of utmost importance for the next generation of kinase inhibitor therapies is the need to define the relevant patient population for clinical trials and receipt of therapy through molecular characterization of the tumor (Sawyers, Nature 432(18):294-297, 2004). Overcoming this barrier will require the development and widespread adoption of appropriate molecular diagnostic assays.

Another complicating aspect of human cancers, especially solid tumors, is the pronounced heterogeneity of both neoplastic and normal cells on the histological, genetic, and/or gene expression levels (Heppner, Cancer Res. 44:2259-2265, 1984; Loeb et al., Proc. Natl. Acad. Sci. USA 100:776-781, 2003). Tumor heterogeneity presents challenges in, among other things, the study of the mechanisms of cancer development and the development of therapeutics to eradicate cancer cells. As a result, the field of molecular diagnostics has turned, in some cases, to the discovery of combinations of biomarkers that make up a molecular “signature” of a given disease phenotype. Such signatures may range from combinations of 2 or 3 biomarkers to combinations of 10, 25, 50 or even more biomarkers.

To realize the broader potential of targeted cancer therapy, there is a need for diagnostic tests and methods to detect oncogenic mutations and molecular signatures implicated in the onset and progression of human cancers. Such methods and diagnostic tests will, among other things, facilitate the screening of new drugs that inhibit such mutant/fusion proteins as well as new methods to select patients for therapy and monitor the responsiveness of patients and their tumors to such therapy.

### SUMMARY

Disclosed herein are methods of predicting response of a tumor to an ALK inhibitor. Also disclosed are methods of diagnosing a subject with a tumor or determining the prognosis of a subject with a tumor. The methods include detecting presence of an ALK gene fusion (such as EML4-ALK, TFG-ALK, KIF5B-ALK, or combinations thereof) in a sample from a subject. In some examples, presence of an ALK gene fusion indicates that the tumor is predicted to respond to an ALK inhibitor. In other examples, presence of an ALK gene fusion indicates the presence of a tumor (such as a lung tumor, a head and neck tumor, a breast tumor, a gastric tumor, or a lymphoma) in the subject. In further examples, presence of an ALK gene fusion indicates that the subject has a poor prognosis. In particular embodiments, presence of an ALK gene fusion in the sample is detected with a quantitative nuclease protection assay and microarray.

Also disclosed herein are arrays for detecting the presence of ALK and/or ROS gene fusions in a sample. In some embodiments, the array can include a surface having spatially discrete regions, each region including an anchor attached to the surface (e.g., stably, covalently, reversibly, or irreversibly attached to the surface) and a bifunctional linker which has a first portion complementary to the anchor and a second portion complementary to a target nucleic acid. In some embodiments, the target nucleic acid includes one or more ALK or ROS gene fusions.

The foregoing and other features of the disclosure will become more apparent from the following detailed description, which proceeds with reference to the accompanying FIGURE.

### BRIEF DESCRIPTION OF THE DRAWING

FIG. 1 is a schematic diagram showing full-length wild type EML4 and ALK genes, an exemplary EML4-ALK fusion gene, and exemplary ALK flanking probes and an exemplary fusion probe. The EML4-ALK fusion gene includes a 5′ portion of EML4 and a 3′ portion of ALK. The flanking 5′-ALK probe and 3′-ALK probe hybridize to full-length ALK and are detected following nuclease treatment. The flanking 3′-ALK probe also hybridizes to the fusion gene and is detected following nuclease treatment; however the flanking 5′-ALK probe does not hybridize to the fusion gene and is hydrolyzed by nuclease treatment. A fusion probe spanning the fusion point can also optionally be included in the assay. When the EML4-ALK gene fusion is present in a sample, the fusion probe hybridizes and is detected following nuclease treatment (solid line). When the gene fusion is not present in a sample, the fusion probe only partially hybridizes to EML4 and ALK and at least the non-hybridized portion is hydrolyzed by the nuclease treatment (dotted lines).

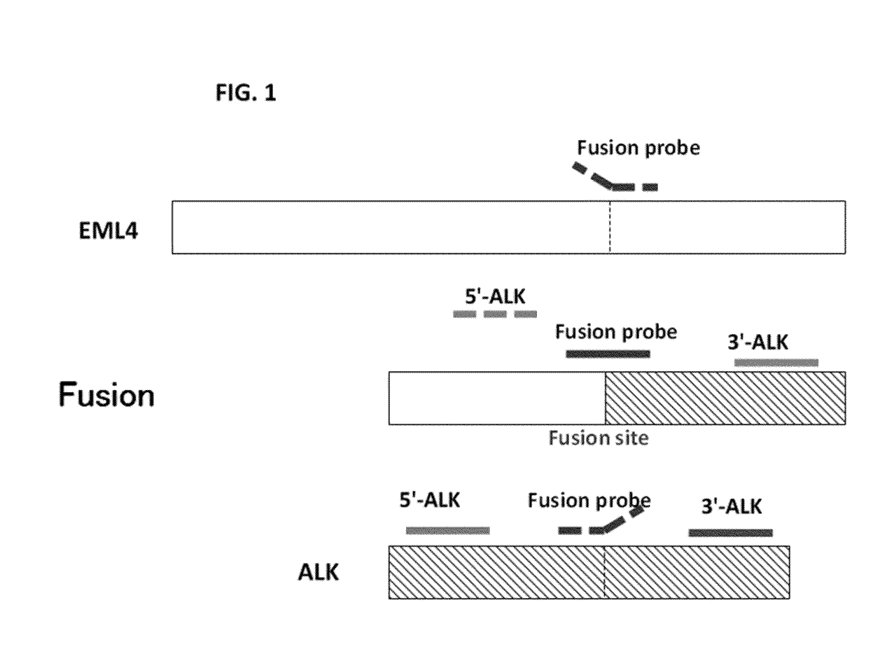

### SEQUENCES

Any nucleic acid and amino acid sequences listed herein are shown using standard letter abbreviations for nucleotide bases, and three letter code for amino acids, as defined in 37 C.F.R. 1.822. In at least some cases, only one strand of each nucleic acid sequence is shown, but the complementary strand is understood as included by any reference to the displayed strand. The Sequence Listing is submitted as an ASCII text file in the form of the file named “Sequence.txt” (˜88 kb), which was created on Apr. 3, 2013, and which is incorporated by reference herein. In the provided sequences:

SEQ ID NOs: 1-8 are exemplary EML4-ALK gene fusion variant nucleic acid sequences.

SEQ ID NO: 9 is an exemplary TFG-ALK gene fusion nucleic acid sequence.

SEQ ID NO: 10 is an exemplary KIF5B-ALK gene fusion nucleic acid sequence.

SEQ ID NO: 11 is an exemplary full-length ALK nucleic acid sequence.

SEQ ID NO: 12 is an exemplary full-length EML4 nucleic acid sequence.

SEQ ID NO: 13 is an exemplary SLC34A2(e4)—ROS(e32) gene fusion nucleic acid sequence.

SEQ ID NO: 14 is an exemplary SLC34A2(e13)—ROS(e32) gene fusion nucleic acid.

SEQ ID NO: 15 is an exemplary CD74(e6)—ROS(e34) gene fusion nucleic acid sequence.

SEQ ID NO: 16 is an exemplary full-length ROS1 nucleic acid sequence.

SEQ ID NOs: 17-39 are exemplary ALK and ROS fusion probe and flanking probe nucleic acid sequences.

SEQ ID NOs: 40-43 are exemplary control gene probe nucleic acid sequences.

SEQ ID NOs: 44-66 are exemplary ALK and ROS array programming linker nucleic acid sequences.

SEQ ID NOs: 67-70 are exemplary control gene programming linker nucleic acid sequences.

SEQ ID NOs: 71-93 are exemplary ALK and ROS array detection linker nucleic acid sequences.

SEQ ID NOs: 94-97 are exemplary control detection linker nucleic acid sequences.

### DETAILED DESCRIPTION

### I. Abbreviations

ALK: anaplastic lymphoma kinase

CD74: CD74 antigen

EML4: echinoderm microtubule associated protein like 4

EZR: ezrin

FFPE: formalin-fixed paraffin-embedded

KIF5B: kinesin family member 5B

ROS (or ROS1): c-ros oncogene 1

SDC4: syndecan 4

SLC34A2: Solute carrier family 34 member 2

TFG: TRK-fused gene

TPM3: tropomyosin 3

### II. Terms

Unless otherwise noted, technical terms are used according to conventional usage. Definitions of common terms in molecular biology may be found in Benjamin Lewin, Genes VII, published by Oxford University Press, 2000 (ISBN 019879276X); Kendrew et al. (eds.), The Encyclopedia of Molecular Biology, published by Blackwell Publishers, 1994 (ISBN 0632021829); Robert A. Meyers (ed.), Molecular Biology and Biotechnology: a Comprehensive Desk Reference, published by Wiley, John & Sons, Inc., 1995 (ISBN 0471186341); and George P. Rédei, Encyclopedic Dictionary of Genetics, Genomics, and Proteomics, 2nd Edition, 2003 (ISBN: 0-471-26821-6).

The following explanations of terms and methods are provided to better describe the present disclosure and to guide those of ordinary skill in the art to practice the present disclosure. The singular forms “a,” “an,” and “the” refer to one or more than one, unless the context clearly dictates otherwise. For example, the term “comprising a cell” includes single or plural cells and is considered equivalent to the phrase “comprising at least one cell.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

All publications, patent applications, patents, and other references mentioned herein are incorporated by reference in their entirety for all purposes. All sequences associated with the GenBank Accession Nos. mentioned herein are incorporated by reference in their entirety as were present on Apr. 27, 2012, to the extent permissible by applicable rules and/or law. In case of conflict, the present specification, including explanations of terms, will control.

Although methods and materials similar or equivalent to those described herein can be used to practice or test the disclosed technology, suitable methods and materials are described below. The materials, methods, and examples are illustrative only and not intended to be limiting.

To facilitate review of the various embodiments of this disclosure, the following explanations of specific terms are provided:

Anaplastic lymphoma kinase (ALK): A receptor tyrosine kinase belonging to the insulin receptor superfamily. The ALK protein includes an extracellular domain, a transmembrane domain and an intracellular kinase domain.

Nucleic acid and protein sequences for ALK are publicly available. For example, GenBank Accession No. NM_{—}004304 discloses an exemplary human ALK nucleic acid sequence, and GenBank Accession No. NP_{—}004295 discloses an exemplary ALK protein sequence, both of which are incorporated by reference as provided by GenBank on Apr. 27, 2012.

CD74 antigen (CD74): An integral membrane protein that functions as a MHC Class II chaperone. It is also a receptor for macrophage migration inhibitory factor.

Nucleic acid and protein sequences for CD74 are publicly available. For example, GenBank Accession Nos. NM_{—}001025158, NM_{—}004355, and NM_{—}001025159 disclose exemplary human CD74 nucleic acid sequences, and GenBank Accession Nos. NP_{—}001020329, NP_{—}004346, and NP_{—}001020330 disclose exemplary CD74 protein sequences, each of which are incorporated by reference as provided by GenBank on Apr. 27, 2012.

Complementary: Able to form base pairs between nucleic acids. Oligonucleotides and their analogs hybridize by hydrogen bonding, which includes Watson-Crick, Hoogsteen, or reversed Hoogsteen hydrogen bonding, between complementary bases. Generally, nucleic acid molecules consist of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as “base pairing.” More specifically, A will hydrogen bond to T or U, and G will bond to C. “Complementary” refers to the base pairing that occurs between two distinct nucleic acids or two distinct regions of the same nucleic acid.

“Specifically hybridizable” and “specifically complementary” are terms that indicate a sufficient degree of complementarity such that stable and specific binding occurs between a probe (or its analog) and a nucleic acid target (e.g., DNA or RNA). The probe or analog may, but need not have 100% complementarity to its target sequence to be specifically hybridizable. A probe or analog is specifically hybridizable when there is a sufficient degree of complementarity to avoid non-specific binding of the probe or analog to non-target sequences under conditions where specific binding is desired, for example in the methods disclosed herein. Such binding is referred to as specific hybridization.

Contact: Placement in direct physical association; includes both in solid and liquid form. For example, contacting can occur in vitro with a nucleic acid probe and biological sample in solution or on a surface.

Detect: To determine if an agent (such as a signal, particular nucleotide, amino acid, nucleic acid molecule, polypeptide, and/or organism) is present or absent, for example a gene fusion nucleic acid. In some examples, this can further include quantification. For example, use of the disclosed methods and probes in particular examples permits detection of a gene fusion in a sample.

Detectable label: A compound or composition that is conjugated directly or indirectly to another molecule (such as a nucleic acid molecule, for example a fusion probe, a flanking probe, or a detection probe) to facilitate detection of that molecule. Specific, non-limiting examples of labels include fluorescent and fluorogenic moieties, chromogenic moieties, haptens, affinity tags, and radioactive isotopes. The label can be directly detectable (e.g., optically detectable) or indirectly detectable (for example, via interaction with one or more additional molecules that are in turn detectable). Exemplary labels in the context of the probes disclosed herein include haptens (such as biotin, digoxigenin, and dinitrophenyl), enzymes (such as horseradish peroxidase and alkaline phosphatase), and fluorophores (such as fluorescein and phycoerythrin). Methods for labeling nucleic acids, and guidance in the choice of labels useful for various purposes, are discussed, e.g., in Sambrook and Russell, in Molecular Cloning: A Laboratory Manual, 3^{rd} Ed., Cold Spring Harbor Laboratory Press (2001) and Ausubel et al., in Current Protocols in Molecular Biology, Greene Publishing Associates and Wiley-Intersciences (1987, and including updates).

Echinoderm microtubule associated protein like 4 (EML4): A microtubule-associated WD-repeat protein belonging to the family of EMAP-like proteins. EML4 colocalizes with and stabilizes microtubules.

Nucleic acid and protein sequences for EML4 are publicly available. For example, GenBank Accession Nos. NM_{—}019063 and NM_{—}001145076 disclose exemplary human EML4 nucleic acid sequences, and GenBank Accession Nos. NP_{—}061936 and NP_{—}001138548 disclose exemplary EML4 protein sequences, each of which are incorporated by reference as provided by GenBank on Apr. 27, 2012.

Ezrin (EZR): A cytoplasmic peripheral membrane protein. It is a protein-tyrosine kinase substrate and is an intermediate between the plasma membrane and the actin cytoskeleton.

Nucleic acid and protein sequences for EZR are publicly available. For example, GenBank Accession Nos. NM_{—}001111077 and NM_{—}003379 disclose exemplary human EZR nucleic acid sequences, and GenBank Accession Nos. NP_{—}001104547 and NP_{—}003370 disclose exemplary EZR protein sequences, each of which are incorporated by reference as provided by GenBank on Apr. 27, 2012.

Gene Fusion: A hybrid gene formed from two or more previously separate genes. Gene fusions can occur as the result of a chromosomal rearrangement, such as a translocation, interstitial deletion, or chromosomal inversion. The “fusion point” or “breakpoint” of a gene fusion is the point of transition between the sequence from the first gene in the fusion to the sequence from the second gene in the fusion.

The terms “gene fusion” and “fusion gene” are used interchangeably herein and indicate the products of a chromosomal rearrangement, including but not limited to DNA (such as genomic DNA or cDNA), RNA, (including mRNA), or protein. In particular examples a gene fusion includes one or more RNAs.

Hybridization: The ability of complementary single-stranded DNA, RNA, or DNA/RNA hybrids to form a duplex molecule (also referred to as a hybridization complex). Nucleic acid hybridization techniques can be used to form hybridization complexes between a nucleic acid probe, and the gene it is designed to target.

Hybridization conditions resulting in particular degrees of stringency will vary depending upon the nature of the hybridization method and the composition and length of the hybridizing nucleic acid sequences. Generally, the temperature of hybridization and the ionic strength (such as the Na⁺ concentration) of the hybridization buffer will determine the stringency of hybridization. Calculations regarding hybridization conditions for attaining particular degrees of stringency are discussed in Sambrook et al., (1989) Molecular Cloning, second edition, Cold Spring Harbor Laboratory, Plainview, N.Y. (chapters 9 and 11).

Inhibitor: Any chemical compound, nucleic acid molecule, or peptide (such as an antibody), specific for a gene product that can reduce activity of a gene product (such as ALK).

Kinesin family member 5B (KIF5B): An N-kinesin (Plus-end motor) belonging to the superfamily of kinesin-1 molecular motor proteins. KIF5B is implicated in lysosomal and mitochondrial transport.

Nucleic acid and protein sequences for KIF5B are publicly available. For example, GenBank Accession No. NM_{—}004521 discloses an exemplary human KIF5B nucleic acid sequence, and GenBank Accession No. NP_{—}004512 discloses an exemplary KIF5B protein sequence, both of which are incorporated by reference as provided by GenBank on Apr. 27, 2012.

Leucine-rich repeats and immunoglobulin-like domains 1 (LRIG1): A transmembrane protein widely expressed in human tissues that has been shown to interact with receptor tyrosine kinases such as EGFR, MET, and RET.

Nucleic acid and protein sequences for LRIG1 are publicly available. For example, GenBank Accession No. NM_{—}015541 discloses an exemplary human LRIG1 nucleic acid sequence, and GenBank Accession No. NP_{—}056356 discloses an exemplary LRIG1 protein sequence, both of which are incorporated by reference as provided by GenBank on Apr. 27, 2012.

Nuclease: An enzyme that cleaves a phosphodiester bond. An endonuclease is an enzyme that cleaves an internal phosphodiester bond in a nucleotide chain (in contrast to exonucleases, which cleave a phosphodiester bond at the end of a nucleotide chain). Some nucleases have both endonuclease and exonuclease activities. Endonucleases include restriction endonucleases or other site-specific endonucleases (which cleave DNA at sequence specific sites), DNase I, Bal 31 nuclease, S1 nuclease, Mung bean nuclease, Ribonuclease A, Ribonuclease T1, RNase I, RNase PhyM, RNase U2, RNase CLB, micrococcal nuclease, and apurinic/apyrimidinic endonucleases. Exonucleases include exonuclease III and exonuclease VII. In particular examples, a nuclease is specific for single-stranded nucleic acids, such as S1 nuclease, Mung bean nuclease, Ribonuclease A, or Ribonuclease T1.

Nucleic acid: A deoxyribonucleotide or ribonucleotide polymer in either single or double stranded form, and unless otherwise limited, encompassing analogs of natural nucleotides that hybridize to nucleic acids in a manner similar to naturally occurring nucleotides. The term “nucleotide” includes, but is not limited to, a monomer that includes a base (such as a pyrimidine, purine or synthetic analogs thereof) linked to a sugar (such as ribose, deoxyribose or synthetic analogs thereof), or a base linked to an amino acid, as in a peptide nucleic acid (PNA). A “nucleotide” also includes a locked nucleic acid (LNA). A nucleotide is one monomer in a polynucleotide. A nucleotide sequence refers to the sequence of bases in a polynucleotide.

Probe: A nucleic acid molecule that is capable of hybridizing with a target nucleic acid molecule (e.g., genomic DNA, cDNA, RNA, or mRNA target nucleic acid molecule) and, after hybridization to the target, is capable of being detected either directly or indirectly. Thus probes permit the detection, and in some examples quantification, of a target nucleic acid molecule, such as a gene fusion nucleic acid molecule or a nucleic acid molecule that is involved in a gene fusion event. In some examples, a probe includes a detectable label. In some examples, probes can include one or more peptide nucleic acids and/or one or more locked nucleic acids.

A probe is capable of hybridizing with sequences including one or more variations from a “wild type” sequence or portion of a sequence (for example in a gene fusion). For example, a probe may include a sequence having at least 90% identity (such as 90%, 91%, 92%, 93%, 94%, 95%, 96%, 97%, 98%, 99%, or more identity) with a “wild type” gene sequence.

In some examples, a “fusion probe” is a probe that includes nucleic acid sequences capable of hybridizing with sequences from two separate genes when the two genes are part of a gene fusion. A fusion probe includes a 5′ portion capable of hybridizing with a first nucleic acid (for example from a first gene) and a 3′ portion capable of hybridizing with a second nucleic acid (for example, from a second gene), wherein the fusion probe spans the point where the first gene and the second gene are fused (the “fusion point”).

In other examples, a “flanking probe” is a probe that includes nucleic acid sequences capable of hybridizing with a single nucleic acid and located 5′ or 3′ to a fusion point. A 5′ flanking probe includes a probe capable of hybridizing with a portion of a nucleic acid 5′ to a fusion point and a 3′ flanking probe includes a probe capable of hybridizing with a portion of a nucleic acid 3′ to a fusion point.

ROS1: A proto-oncogene, also known as c-ros oncogene 1, ROS and MCF3, which belongs to the sevenless subfamily of tyrosine kinase insulin receptor genes. The ROS1 protein is a type I integral membrane protein with tyrosine kinase activity.

Nucleic acid and protein sequences for ROS1 are publicly available. For example, GenBank Accession Nos. NM_{—}002944, M34353, M13880 and X51619 disclose exemplary human ROS1 nucleic acid sequences, and GenBank Accession Nos. NP_{—}002935, P08922, and AAA60278 disclose exemplary ROS1 protein sequences, each of which are incorporated by reference as provided by GenBank on Apr. 27, 2012.

Sample: A biological specimen containing DNA (for example, genomic DNA or cDNA), RNA (including mRNA), protein, or combinations thereof, obtained from a subject. Examples include, but are not limited to cells, cell lysates, chromosomal preparations, peripheral blood, urine, saliva, tissue biopsy (such as a tumor biopsy or lymph node biopsy), surgical specimen, bone marrow, amniocentesis samples, and autopsy material. In one example, a sample includes RNA, such as mRNA. In particular examples, samples are used directly (e.g., fresh or frozen), or can be manipulated prior to use, for example, by fixation (e.g., using formalin) and/or embedding in wax (such as formalin-fixed paraffin-embedded (FFPE) tissue samples).

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Homologs or orthologs of nucleic acid or amino acid sequences possess a relatively high degree of sequence identity/similarity when aligned using standard methods.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn, and tblastx. Blastn is used to compare nucleic acid sequences, while blastp is used to compare amino acid sequences. Additional information can be found at the NCBI web site.

Once aligned, the number of matches is determined by counting the number of positions where an identical nucleotide or amino acid residue is present in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100.

One indication that two nucleic acid molecules are closely related is that the two molecules hybridize to each other under stringent conditions. Stringent conditions are sequence-dependent and are different under different environmental parameters. The nucleic acid probes disclosed herein are not limited to the exact sequences shown, as those skilled in the art will appreciate that changes can be made to a sequence, and not substantially affect the ability of a probe to function as desired. For example, sequences having at least 80%, at least 85%, at least 90%, at least 91%, at least 92%, at least 93%, at least 94%, at least 95%, at least 96%, at least 97%, at least 98%, or at least 99%, such as 100% sequence identity to the disclosed probes are provided herein. One of skill in the art will appreciate that these sequence identity ranges are provided for guidance only; it is possible that probes can be used that fall outside these ranges.

Solute carrier family 34 member 2 (SLC34A2): A pH-sensitive sodium-dependent phosphate transporter.

Nucleic acid and protein sequences for SLC34A2 are publicly available. For example, GenBank Accession Nos. NM_{—}006424, NM_{—}001177998, and NM_{—}001177999 disclose exemplary human SLC34A2 nucleic acid sequences, and GenBank Accession Nos. NP_{—}006415, NP_{—}001171469, and NP_{—}001171470 disclose exemplary SLC34A2 protein sequences, each of which are incorporated by reference as provided by GenBank on Apr. 27, 2012.

Subject: Living multi-cellular vertebrate organisms, a category that includes human and non-human mammals, such as veterinary subjects. In one example, a subject is known or suspected of having a tumor associated with a gene fusion, such as ALK or ROS.

Syndecan 4 (SDC4): A transmembrane (type I) heparan sulfate proteoglycan that functions as a receptor in intracellular signaling.

Nucleic acid and protein sequences for SDC4 are publicly available. For example, GenBank Accession No. NM_{—}002999 discloses an exemplary human SDC4 nucleic acid sequence, and GenBank Accession Nos. NP002990 discloses an exemplary SDC4 protein sequence, each of which are incorporated by reference as provided by GenBank on Apr. 27, 2012.

Therapeutically effective amount: A quantity of an agent or compound sufficient to achieve a desired effect in a subject or a cell being treated. For instance, this can be the amount necessary to ameliorate a sign or symptom of a disease or disorder, such as cancer.

TRK-fused gene (TFG): A protein including SH2 and SH3 domains and an N-terminal coiled-coiled domain. The C. elegans homolog of TFG suppresses apoptosis and is involved in cell-size control.

Nucleic acid and protein sequences for TFG are publicly available. For example, GenBank Accession Nos. NM_{—}006070 and NM_{—}001007565 disclose exemplary human TFG nucleic acid sequences, and GenBank Accession Nos. NP_{—}006061 and NP_{—}001007566 disclose exemplary TFG protein sequences, each of which are incorporated by reference as provided by GenBank on Apr. 27, 2012.

Tropomyosin 3 (TPM3): A member of the tropomyosin family of actin-binding proteins involved in contractile system of striated and smooth muscle and the cytoskeleton of non-muscle cells.

Nucleic acid and protein sequences for TPM3 are publicly available. For example, GenBank Accession Nos. NM_{—}001043351, NM_{—}001043353, NM_{—}153649, NM_{—}152263, and NM_{—}001043352 disclose exemplary human TPM3 nucleic acid sequences, and GenBank Accession Nos. NP_{—}001036816, NP_{—}001036818, NP_{—}705935, NP_{—}689476, and NP_{—}001036817 disclose exemplary TPM3 protein sequences, each of which are incorporated by reference as provided by GenBank on Apr. 27, 2012.

### III. Methods of Determining Tumor Responsiveness, Diagnosis, or Prognosis

Disclosed herein are methods of detecting one or more ALK or ROS fusions or wild type genes (such as EML4-ALK variant 1, EML4-ALK variant 2, EML4-ALK variant 3a, EML4-ALK variant 3b, EML4-ALK variant 4, EML4-ALK variant 5a, EML4-ALK variant 5b, EML4-ALK variant 6, EML4 wild type, ALK, TFG-ALK, KIF5B-ALK, EZR(e9)-ROS(e34), LRIG1(e16)-ROS(e35), SLC34A2(e4)-ROS(e32), SLC34A2(e13)-ROS(e32), CD74(e6)-ROS(e32), CD74(e6)-ROS(e34), SDC(e2)-ROS(e32), TPM(e8)-ROS(e35), or ROS1) in a biological sample, for example utilizing the arrays and methods disclosed below. Also disclosed herein are methods of determining diagnosis or prognosis of a subject with a tumor or predicting response of a tumor in a subject to treatment with a therapeutically effective amount of an anaplastic lymphoma kinase (ALK) inhibitor. The methods can include detecting presence of one or more ALK gene fusions (such as an EML4-ALK, TFG-ALK, or KIF5B-ALK gene fusion) in a sample from the subject.

The samples of use in the disclosed methods include any specimen that includes nucleic acid (such as genomic DNA, cDNA, viral DNA or RNA, rRNA, tRNA, mRNA, oligonucleotides, nucleic acid fragments, modified nucleic acids, synthetic nucleic acids, or the like). In particular examples, the sample includes mRNA. In some examples, the disclosed methods include obtaining a sample (e.g., obtaining the sample from the subject) prior to processing and/or analysis of the sample. In some examples, the disclosed methods include selecting a subject having a tumor (such as a lung tumor, a gastric tumor, a breast tumor, a head and neck tumor, or a lymphoma).

Appropriate samples include any conventional biological samples, including clinical samples obtained from a human or veterinary subject. Exemplary samples include, without limitation, cells, cell lysates, blood smears, cytocentrifuge preparations, cytology smears, bodily fluids (e.g., blood, plasma, serum, saliva, sputum, urine, bronchoalveolar lavage, semen, etc.), tissue biopsies (e.g., tumor biopsies), fine-needle aspirates, and/or tissue sections (e.g., cryostat tissue sections and/or paraffin-embedded tissue sections). In other examples, the sample includes circulating tumor cells. In particular examples, samples are used directly (e.g., fresh or frozen), or can be manipulated prior to use, for example, by fixation (e.g., using formalin) and/or embedding in wax (such as FFPE tissue samples).

Methods for detecting the presence of one or more gene fusions (such as one or more of an EML4-ALK, TFG-ALK, and KIF5B-ALK gene fusion) are known to one of skill in the art and include in situ hybridization (such as fluorescence in situ hybridization, colorimetric in situ hybridization, and silver in situ hybridization), sequencing, and PCR-based methods (such as RT-PCR or real-time RT-PCR). Additional methods include microarray or bead-based assays.

In some embodiments, the methods can include contacting a sample from a subject (such as a sample including nucleic acids, for example a tumor sample) with a fusion probe that has a 5′ portion complementary to a first nucleic acid (including but not limited to EML4, TFG, or KIF5B) and a 3′ portion complementary to a second nucleic acid (including but not limited to ALK) wherein the fusion probe spans a fusion point of the first nucleic acid and the second nucleic acid. The fusion probe is incubated with the sample under conditions sufficient for the fusion probe to specifically hybridize to a gene fusion. The sample is contacted with a nuclease specific for single-stranded nucleic acids (for example, S1 nuclease), and the presence of the fusion probe detected. The fusion gene is identified as present in the sample when the fusion probe is detected. In particular examples, the first nucleic acid and the second nucleic acid are mRNA (for example, the gene fusion nucleic acid detected is mRNA). Particular gene fusions and exemplary fusion probes are described in Section IV, below.

In additional embodiments, the methods can include contacting a sample from a subject with a first probe complementary to a first nucleic acid (such as ALK) 5′ to a fusion point between the first nucleic acid and a second nucleic acid under conditions sufficient for the first probe to specifically hybridize to the first nucleic acid, contacting the sample with a second probe complementary to the first nucleic acid 3′ to the fusion point between the first and second nucleic acids under conditions sufficient for the second probe to specifically hybridize to the first nucleic acid, contacting the sample with a nuclease specific for single-stranded nucleic acids (for example, S1 nuclease), detecting presence of the first probe and the second probe, and determining a ratio of the first probe to the second probe (or the ratio of the second probe to the first probe). The fusion gene is identified as present in the sample when the ratio of the first probe to the second probe (or the ratio of the second probe to the first probe) is different from one (for example, statistically significantly different from one).

In some examples, the gene fusion is detected and does not include a 3′ portion of the first nucleic acid if the ratio of the first probe to the second probe is greater than one (for example, statistically significantly greater than one). In other examples, the gene fusion is detected and does not include a 5′ portion of the first nucleic acid if the ratio of the first probe to the second probe is less than one (for example, statistically significantly less than one). In further examples, the gene fusion is detected and does not include a 5′ portion of the first nucleic acid if the ratio of the second probe to the first probe is greater than one (for example, statistically significantly greater than one). In other examples, the gene fusion is detected and does not include a 3′ portion of the first nucleic acid if the ratio of the second probe to the first probe is less than one (for example, statistically significantly less than one). In particular examples, the first nucleic acid and the second nucleic acid are mRNA (for example, the gene fusion nucleic acid detected is mRNA). Particular wild type genes and exemplary flanking probes are described in Section IV, below.

In particular embodiments of the disclosed methods, the presence of gene fusions are detected in the sample utilizing a quantitative nuclease protection assay and array (such as an array described in Section V, below). The quantitative nuclease protection assay is described in International Patent Publications WO 99/032663; WO 00/037683; WO 00/037684; WO 00/079008; WO 03/002750; and WO 08/121,927; and U.S. Pat. Nos. 6,238,869; 6,458,533; and 7,659,063, each of which is incorporated herein by reference in their entirety. See also, Martel et al, Assay and Drug Development Technologies. 2002, 1 (1-1):61-71; Martel et al, Progress in Biomedical Optics and Imaging, 2002, 3:35-43; Martel et al, Gene Cloning and Expression Technologies, Q. Lu and M. Weiner, Eds., Eaton Publishing, Natick (2002); Seligmann, B. PharmacoGenomics, 2003, 3:36-43; Martel et al, “Array Formats” in “Microarray Technologies and Applications,” U. R. Muller and D. Nicolau, Eds, Springer-Verlag, Heidelberg; Sawada et al, Toxicology in Vitro, 20:1506-1513; Bakir, et al, Biorg. & Med. Chem. Lett, 17: 3473-3479; Kris, et al, Plant Physiol. 144: 1256-1266; Roberts, et al, Laboratory Investigation, 87: 979-997; Rimsza, et al, Blood, 2008 October 15, 112 (8): 3425-3433; Pechhold, et al, Nature Biotechnology, 27, 1038-1042. All of these are fully incorporated by reference herein.

The samples described herein can be prepared using any method now known or hereafter developed in the art. In some examples, cells in the sample are lysed or permeabilized in an aqueous solution (for example using a lysis buffer). The aqueous solution or lysis buffer includes detergent (such as sodium dodecyl sulfate (SDS)) and one or more chaotropic agents (such as formamide, guanidinium HCl, guanidinium isothiocyanate, or urea). The solution may also contain a buffer (for example SSC). In some examples, the lysis buffer includes about 15% to 25% formamide (v/v), about 0.01% to 0.1% SDS, and about 0.5-6×SSC. The buffer may optionally include tRNA (for example, about 0.001 to about 2.0 mg/ml) or a ribonuclease. The lysis buffer may also include a pH indicator, such as Phenol Red. In a particular example, the lysis buffer includes 20% formamide, 3×SSC (79.5%), 0.05% SDS, 1 μg/ml tRNA, and 1 mg/ml Phenol Red. Cells are incubated in the aqueous solution for a sufficient period of time (such as about 1 minute to about 60 minutes, for example about 5 minutes to about 20 minutes, or about 10 minutes) and at a sufficient temperature (such as about 22° C. to about 115° C., for example, about 37° C. to about 105° C., or about 90° C. to about 110° C.) to lyse or permeabilize the cell. In some examples, lysis is performed at about 95° C., if the gene fusion nucleic acid to be detected is RNA. In other examples, lysis is performed at about 105° C., if the gene fusion nucleic acid to be detected is DNA.

In some examples, nucleic acid (e.g., a flanking or fusion probe, such as a nucleic acid probe comprising any one of SEQ ID NOs: 17-43) can be added to a sample at a concentration ranging from about 10 μM to about 10 nM (such as about 30 μM to 5 nM, about 100 μM to about 1 nM), in a buffer such as, for example, 6×SSPE-T (0.9 M NaCl, 60 mM NaH₂PO₄, 6 mM EDTA, and 0.05% Triton X-100) or lysis buffer (described above). In one example, the probe is added to the sample at a final concentration of about 30 μM. In another example, the probe is added to the sample at a final concentration of about 167 μM. In a further example, the probe is added to the sample at a final concentration of about 1 nM.

The nucleic acids in the sample are denatured (for example at about 95° C. to about 105° C. for about 5-15 minutes) and hybridized to a probe for between about 10 minutes and about 24 hours (for example, at least about 1 hour to 20 hours, or about 6 hours to 16 hours) at a temperature ranging from about 4° C. to about 70° C. (for example, about 37° C. to about 65° C., about 45° C. to about 60° C., or about 50° C. to about 60° C.). In some examples, the probes are incubated with the sample at a temperature of at least about 40° C., at least about 45° C., at least about 50° C., at least about 55° C., at least about 60° C., at least about 65° C., or at least about 70° C. In one example, the probes are incubated with the sample at about 60° C. In another example, the probes are incubated with the sample at about 50° C. These hybridization temperatures are exemplary, and one of skill in the art can select appropriate hybridization temperature depending on factors such as the length and nucleotide composition of the probes.

In some embodiments, the methods do not include nucleic acid purification (for example, nucleic acid purification is not performed prior to contacting the sample with the probes and/or nucleic acid purification is not performed following contacting the sample with the probes). In some examples, no pre-processing of the sample is required except for cell lysis. In some examples, cell lysis and contacting the sample with the probes occur sequentially, in some non-limiting examples without any intervening steps. In other examples, cell lysis and contacting the sample with the probes occur concurrently.

Following hybridization of the one or more probes and nucleic acids in the sample, the sample is subjected to a nuclease protection procedure. Probes which have hybridized to a full-length nucleic acid or a gene fusion are not hydrolyzed by the nuclease and can be subsequently detected.

Treatment with one or more nucleases will destroy nucleic acid molecules other than the probes which have hybridized to a full-length or gene fusion nucleic acid molecules present in the sample. For example, if the sample includes a cellular extract or lysate, unwanted nucleic acids, such as genomic DNA, cDNA, tRNA, rRNA and mRNAs other than the gene or gene fusion of interest, can be substantially destroyed in this step. Any of a variety of nucleases can be used, including, pancreatic RNAse, mung bean nuclease, S1 nuclease, RNAse A, Ribonuclease T1, Exonuclease III, Exonuclease VII, RNAse CLB, RNAse PhyM, RNAse U2, or the like, depending on the nature of the hybridized complexes and of the undesirable nucleic acids present in the sample. In a particular example, the nuclease is specific for single-stranded nucleic acids, for example S1 nuclease. An advantage of using a nuclease specific for single-stranded nucleic acids in some method embodiments disclosed here is to remove such single-stranded (“sticky”) molecules from subsequent reaction steps where they may lead to unnecessary background or cross-reactivity. S1 nuclease is commercially available from for example, Promega, Madison, Wis. (cat. no. M5761); Life Technologies/Invitrogen, Carlsbad, Calif. (cat. no. 18001-016); Fermentas, Glen Burnie, Md. (cat. no. EN0321), and others. Reaction conditions for these enzymes are well-known in the art and can be optimized empirically.

In some examples, S1 nuclease diluted in an appropriate buffer (such as a buffer including sodium acetate, sodium chloride, zinc sulfate, and detergent, for example, 0.25 M sodium acetate, pH 4.5, 1.4 M NaCl, 0.0225 M ZnSO₄, 0.05% KATHON) is added to the hybridized probe mixture and incubated at about 50° C. for about 30-120 minutes (for example, about 60-90 minutes) to digest non-hybridized nucleic acid and unbound probe.

The samples optionally are treated to otherwise remove non-hybridized material and/or to inactivate or remove residual enzymes (e.g., by phenol extraction, precipitation, column filtration, etc.). In some examples, the samples are optionally treated to dissociate the target nucleic acid (such as target gene fusion or target full length or wild type gene) from the probe (e.g., using base hydrolysis and heat). After hybridization, the hybridized target can be degraded, e.g., by nucleases or by chemical treatments, leaving the probes in direct proportion to how much probe had been hybridized to target. Alternatively, the sample can be treated so as to leave the (single strand) hybridized portion of the target, or the duplex formed by the hybridized target and the probe, to be further analyzed.

The presence of the probes in the sample is then detected. In some examples, presence of a fusion probe indicates presence of the corresponding gene fusion in the sample. In other examples, a ratio of probes flanking a fusion point in a full-length gene is determined (for example a ratio of ALK 3′ and 5′ probes). The presence of a gene fusion in the sample is detected if the ratio of the 5′ flanking probe to the 3′ flanking probe or the ratio of the 3′ flanking probe to the 5′ flanking probe is different from one (for example, statistically significantly different from one).

In some examples, the first and second probes are complementary to the 3′ gene in the fusion (for example, ALK). In this example, the gene fusion is detected and does not include a 5′ portion of the nucleic acid if the ratio of the 3′ probe to the 5′ probe is greater than one (for example, statistically significantly greater than one). In some examples, the gene fusion is present and does not include a 5′ portion of ALK if the ratio of 3′-ALK probe to 5′-ALK probe is at least 1.1, such as at least 1.5, at least 1.8, at least 2, at least 2.5, at least 3, at least 4, at least 5, at least 10 or at least 20, for example 1.1 to 20 or 1.1 to 60, such as about 1.1, 1.2, 1.3, 1.4, 1.5, 1.6, 1.7, 1.8, 1.9, 2.0, 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 18, 19, 20, 30, 40, 50, 60, or more. In other examples, the gene fusion is detected and does not include a 3′ portion of ALK if the ratio of the 3′-ALK probe to 5′-ALK probe is less than one (for example, statistically significantly less than one). In some examples, the gene fusion is present and does not include a 3′ portion of the nucleic acid if the ratio is no more than 0.95, such as no more than 0.9, no more than 0.8, no more than 0.7, no more than 0.6, no more than 0.5, or no more than 0.1, for example 0.05 to 0.95, such as about 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.05, or less.

In some embodiments, the gene fusion is present if the ratio of the flanking probes (for example, the ratio of a 5′ flanking probe to a 3′ flanking probe or the ratio of a 3′ flanking probe to a 5′ flanking probe) differs from a control (such as an average ratio in a wild-type sample) by at least two standard deviations (for example, at least 2, 3, 4, 5, or more standard deviations). In some examples, the control is the ratio (for example the average ratio) of flanking probes in a sample or a population of samples that does not include a gene fusion (such as a sample that includes only full-length or wild-type gene, for example, ALK).

A. Predicting Tumor Responsiveness

Disclosed herein are methods of predicting response of a tumor in a subject to treatment with a therapeutically effective amount of an ALK inhibitor. The methods can include detecting presence of one or more ALK gene fusions (such as an EML4-ALK, TFG-ALK, or KIF5B-ALK gene fusion) in a sample from the subject; and identifying the tumor as responsive to an ALK inhibitor if an ALK fusion (such as EML4-ALK, TFG-ALK, KIF5B-ALK, or a combination of two or more thereof) is present in the sample. In some embodiments, the presence of one or more ALK gene fusions is determined utilizing the methods and arrays disclosed herein.

In some embodiments, the tumor is predicted to be responsive to an ALK inhibitor (such as a di(arylamino) aryl ALK inhibitor or a diamino heterocyclic carboxamide ALK inhibitor) if presence of an ALK fusion is detected in a sample from a subject (such as a tumor sample). In some examples, the ALK inhibitor is selected from the compounds disclosed in U.S. Pat. Publication No. 2010/0099658 or International PCT Publication No. WO 10/128,659, both of which are incorporated by reference herein in their entirety. In a particular example, the ALK inhibitor is ASP3026 (Astellas Pharma, Inc.).

In particular examples, the disclosed methods can be used to predict the response to an ALK inhibitor of a lung tumor (for example, non-small cell lung carcinoma or small cell lung carcinoma), a head and neck tumor, a breast tumor, a gastric tumor, or a lymphoma. Presence of an ALK fusion indicates that the tumor is predicted to respond to an ALK inhibitor.

In some embodiments, the disclosed methods can further include administering an ALK inhibitor, such as a di(arylamino) aryl ALK inhibitor or a diamino heterocyclic carboxamide ALK inhibitor (for example, ASP3026) to a subject if an ALK gene fusion is detected in a sample (such as a tumor sample) from the subject. Methods and dosages of ALK inhibitors that can be used are known in the art and can be routinely determined by a skilled clinician (see, e.g., U.S. Pat. Publ. No. 2010/0099658, 2008/0300273 and PCT Publ. No. WO 10/128,659).

B. Diagnosis and Prognosis

Disclosed herein are methods of determining a diagnosis or a prognosis of a subject with a tumor. In some examples, the disclosed methods include determining a diagnosis or prognosis of a subject with a lung tumor (for example, non-small cell lung carcinoma or small cell lung carcinoma), a head and neck tumor, a breast tumor, a gastric tumor, or a lymphoma. The methods can include detecting presence of one or more ALK gene fusions (such as an EML4-ALK, TFG-ALK, or KIF5B-ALK gene fusion, or a combination of two or more thereof) in a sample from the subject. In some embodiments, the presence of one or more ALK gene fusions is determined utilizing the methods and arrays disclosed herein.

In some embodiments of the disclosed methods, presence of an ALK fusion (such as EML4-ALK, TFG-ALK, KIF5B-ALK, or a combination of two or more thereof) in the sample from a subject indicates presence of a malignant tumor in the subject. In other examples, absence of an ALK gene fusion in the sample from the subject indicates a benign (e.g., non-malignant) tumor is present or no tumor is present in the subject.

In other embodiments of the disclosed methods, presence of an ALK gene fusion in a sample from a subject (for example, a tumor sample from the subject) indicates a poor prognosis. In particular examples, presence of an EML4-ALK, TFG-ALK, and/or KIF5B-ALK gene fusion indicates a poor prognosis. For example, presence of an ALK gene fusion in the sample from the subject indicates a poor prognosis, such as a decreased chance of survival (for example decreased overall survival, relapse-free survival, or metastasis-free survival). In an example, a decreased chance of survival includes a survival time of equal to or less than 60 months, such as 50 months, 40 months, 30 months, 20 months, 12 months, 6 months, or 3 months from time of diagnosis or first treatment. In other examples, absence of an ALK gene fusion in the sample indicates a good prognosis (such as increased chance of survival, for example increased overall survival, relapse-free survival, or metastasis-free survival). In an example, an increased survival, relapse-free survival, or metastasis-free survival includes a survival time, relapse-free survival time, or metastasis-free survival time of at least at least 5 years, at least 7 years, or at least 10 years, from time of diagnosis or first treatment.

Poor prognosis can refer to any negative clinical outcome, such as, but not limited to, a decrease in likelihood of survival (such as overall survival, relapse-free survival, or metastasis-free survival), a decrease in the time of survival (e.g., less than 5 years, or less than one year), presence of a malignant tumor, an increase in the severity of disease, a decrease in response to therapy, an increase in tumor recurrence, an increase in metastasis, or the like. In particular examples, a poor prognosis is a decreased chance of survival (for example, a survival time of equal to or less than 60 months, such as 50 months, 40 months, 30 months, 20 months, 12 months, 6 months or 3 months from time of diagnosis or first treatment).

### IV. Gene Fusions and Probes

In some embodiments, the disclosed methods and arrays include detecting presence of one or more EML4-ALK, TFG-ALK, or KIF5B-ALK gene fusions in a sample from a subject (see, e.g., Rikova et al., Cell 131:1190-1203, 2007). Exemplary nucleic acid sequences of ALK fusions detected in at least some embodiments are as follows:

EML4-ALK variant 1 (3180nt)
EML4 exons1-13 + ALK exons 20-30
(SEQ ID NO: 1)
ATGGACGGTTTCGCCGGCAGTCTCGATGATAGTATTTCTGCTGCAAGTACTTCTGATGTTCAAGATCGCC
TGTCAGCTCTTGAGTCACGAGTTCAGCAACAAGAAGATGAAATCACTGTGCTAAAGGCGGCTTTGGCTGA
TGTTTTGAGGCGTCTTGCAATCTCTGAAGATCATGTGGCCTCAGTGAAAAAATCAGTCTCAAGTAAAGGC
CAACCAAGCCCTCGAGCAGTTATTCCCATGTCCTGTATAACCAATGGAAGTGGTGCAAACAGAAAACCAA
GTCATACCAGTGCTGTCTCAATTGCAGGAAAAGAAACTCTTTCATCTGCTGCTAAAAGTGGTACAGAAAA
AAAGAAAGAAAAACCACAAGGACAGAGAGAAAAAAAAGAGGAATCTCATTCTAATGATCAAAGTCCACAA
ATTCGAGCATCACCTTCTCCCCAGCCCTCTTCACAACCTCTCCAAATACACAGACAAACTCCAGAAAGCA
AGAATGCTACTCCCACCAAAAGCATAAAACGACCATCACCAGCTGAAAAGTCACATAATTCTTGGGAAAA
TTCAGATGATAGCCGTAATAAATTGTCGAAAATACCTTCAACACCCAAATTAATACCAAAAGTTACCAAA
ACTGCAGACAAGCATAAAGATGTCATCATCAACCAAGAAGGAGAATATATTAAAATGTTTATGCGCGGTC
GGCCAATTACCATGTTCATTCCTTCCGATGTTGACAACTATGATGACATCAGAACGGAACTGCCTCCTGA
GAAGCTCAAACTGGAGTGGGCATATGGTTATCGAGGAAAGGACTGTAGAGCTAATGTTTACCTTCTTCCG
ACCGGGGAAATAGTTTATTTCATTGCATCAGTAGTAGTACTATTTAATTATGAGGAGAGAACTCAGCGAC
ACTACCTGGGCCATACAGACTGTGTGAAATGCCTTGCTATACATCCTGACAAAATTAGGATTGCAACTGG
ACAGATAGCTGGCGTGGATAAAGATGGAAGGCCTCTACAACCCCACGTCAGAGTGTGGGATTCTGTTACT
CTATCCACACTGCAGATTATTGGACTTGGCACTTTTGAGCGTGGAGTAGGATGCCTGGATTTTTCAAAAG
CAGATTCAGGTGTTCATTTATGTGTTATTGATGACTCCAATGAGCATATGCTTACTGTATGGGACTGGCA
GAAGAAAGCAAAAGGAGCAGAAATAAAGACAACAAATGAAGTTGTTTTGGCTGTGGAGTTTCACCCAACA
GATGCAAATACCATAATTACATGCGGTAAATCTCATATTTTCTTCTGGACCTGGAGCGGCAATTCACTAA
CAAGAAAACAGGGAATTTTTGGGAAATATGAAAAGCCAAAATTTGTGCAGTGTTTAGCATTCTTGGGGAA
TGGAGATGTTCTTACTGGAGACTCAGGTGGAGTCATGCTTATATGGAGCAAAACTACTGTAGAGCCCACA

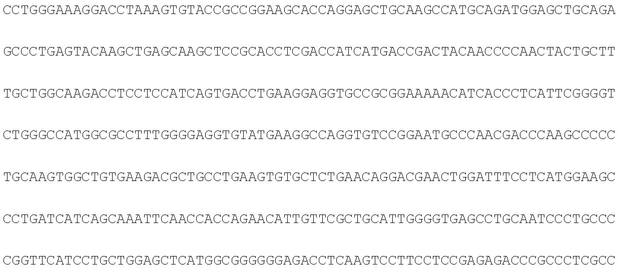

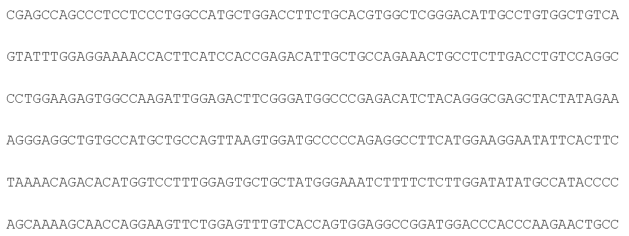

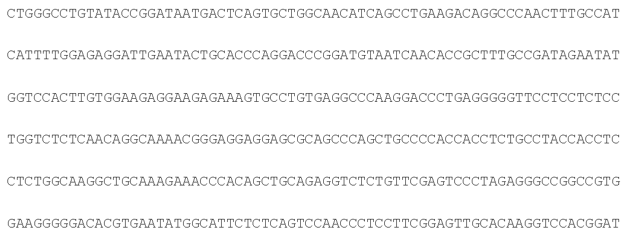

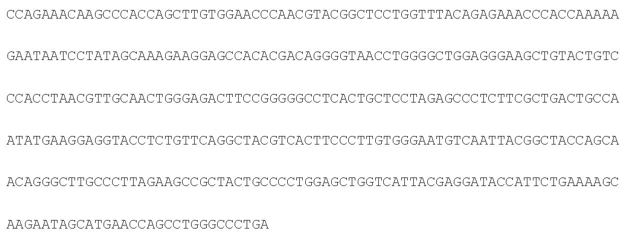

EML4-ALK, variant 2 (3933nt)
EML4 exons 1-20 + ALK exons 20-30
(SEQ ID NO: 2)
ATGGACGGTTTCGCCGGCAGTCTCGATGATAGTATTTCTGCTGCAAGTACTTCTGATGTTCAAGATCGCC
TGTCAGCTCTTGAGTCACGAGTTCAGCAACAAGAAGATGAAATCACTGTGCTAAAGGCGGCTTTGGCTGA
TGTTTTGAGGCGTCTTGCAATCTCTGAAGATCATGTGGCCTCAGTGAAAAAATCAGTCTCAAGTAAAGGC
CAACCAAGCCCTCGAGCAGTTATTCCCATGTCCTGTATAACCAATGGAAGTGGTGCAAACAGAAAACCAA
GTCATACCAGTGCTGTCTCAATTGCAGGAAAAGAAACTCTTTCATCTGCTGCTAAAAGTGGTACAGAAAA
AAAGAAAGAAAAACCACAAGGACAGAGAGAAAAAAAAGAGGAATCTCATTCTAATGATCAAAGTCCACAA
ATTCGAGCATCACCTTCTCCCCAGCCCTCTTCACAACCTCTCCAAATACACAGACAAACTCCAGAAAGCA
AGAATGCTACTCCCACCAAAAGCATAAAACGACCATCACCAGCTGAAAAGTCACATAATTCTTGGGAAAA
TTCAGATGATAGCCGTAATAAATTGTCGAAAATACCTTCAACACCCAAATTAATACCAAAAGTTACCAAA
ACTGCAGACAAGCATAAAGATGTCATCATCAACCAAGAAGGAGAATATATTAAAATGTTTATGCGCGGTC
GGCCAATTACCATGTTCATTCCTTCCGATGTTGACAACTATGATGACATCAGAACGGAACTGCCTCCTGA
GAAGCTCAAACTGGAGTGGGCATATGGTTATCGAGGAAAGGACTGTAGAGCTAATGTTTACCTTCTTCCG
ACCGGGGAAATAGTTTATTTCATTGCATCAGTAGTAGTACTATTTAATTATGAGGAGAGAACTCAGCGAC
ACTACCTGGGCCATACAGACTGTGTGAAATGCCTTGCTATACATCCTGACAAAATTAGGATTGCAACTGG
ACAGATAGCTGGCGTGGATAAAGATGGAAGGCCTCTACAACCCCACGTCAGAGTGTGGGATTCTGTTACT
CTATCCACACTGCAGATTATTGGACTTGGCACTTTTGAGCGTGGAGTAGGATGCCTGGATTTTTCAAAAG
CAGATTCAGGTGTTCATTTATGTGTTATTGATGACTCCAATGAGCATATGCTTACTGTATGGGACTGGCA
GAAGAAAGCAAAAGGAGCAGAAATAAAGACAACAAATGAAGTTGTTTTGGCTGTGGAGTTTCACCCAACA
GATGCAAATACCATAATTACATGCGGTAAATCTCATATTTTCTTCTGGACCTGGAGCGGCAATTCACTAA
CAAGAAAACAGGGAATTTTTGGGAAATATGAAAAGCCAAAATTTGTGCAGTGTTTAGCATTCTTGGGGAA
TGGAGATGTTCTTACTGGAGACTCAGGTGGAGTCATGCTTATATGGAGCAAAACTACTGTAGAGCCCACA
CCTGGGAAAGGACCTAAAGGTGTATATCAAATCAGCAAACAAATCAAAGCTCATGATGGCAGTGTGTTCA
CACTTTGTCAGATGAGAAATGGGATGTTATTAACTGGAGGAGGGAAAGACAGAAAAATAATTCTGTGGGA
TCATGATCTGAATCCTGAAAGAGAAATAGAGGTTCCTGATCAGTATGGCACAATCAGAGCTGTAGCAGAA
GGAAAGGCAGATCAATTTTTAGTAGGCACATCACGAAACTTTATTTTACGAGGAACATTTAATGATGGCT
TCCAAATAGAAGTACAGGGTCATACAGATGAGCTTTGGGGTCTTGCCACACATCCCTTCAAAGATTTGCT
CTTGACATGTGCTCAGGACAGGCAGGTGTGCCTGTGGAACTCAATGGAACACAGGCTGGAATGGACCAGG
CTGGTAGATGAACCAGGACACTGTGCAGATTTTCATCCAAGTGGCACAGTGGTGGCCATAGGAACGCACT
CAGGCAGGTGGTTTGTTCTGGATGCAGAAACCAGAGATCTAGTTTCTATCCACACAGACGGGAATGAACA
GCTCTCTGTGATGCGCTACTCAATAGATGGTACCTTCCTGGCTGTAGGATCTCATGACAACTTTATTTAC
CTCTATGTAGTCTCTGAAAATGGAAGAAAATATAGCAGATATGGAAGGTGCACTGGACATTCCAGCTACA
TCACACACCTTGACTGGTCCCCAGACAACAAGTATATAATGTCTAACTCGGGAGACTATGAAATATTGTA

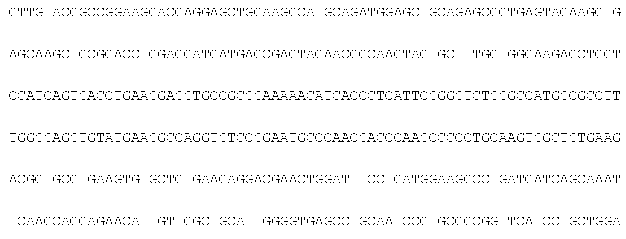

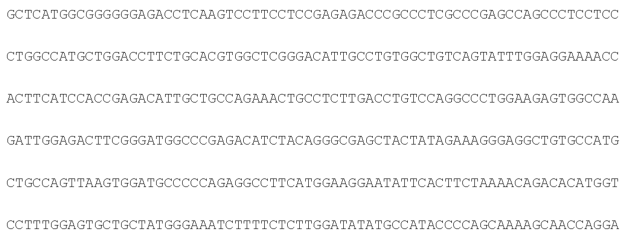

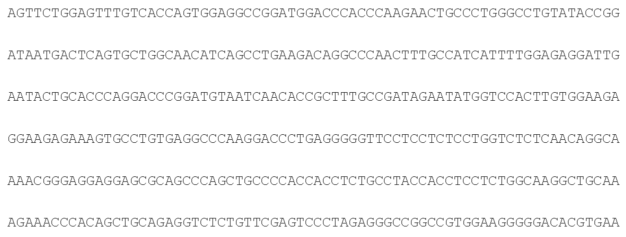

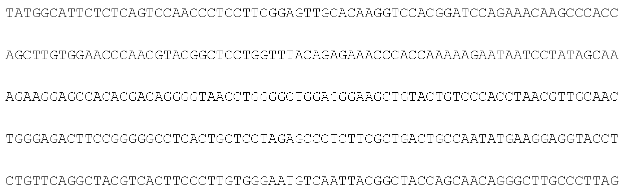

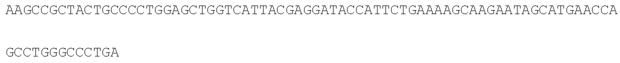

EML4-ALK variant 3a (2358nt)
EML4 exons 1-6 + ALK exons 20-30
(SEQ ID NO: 3)
ATGGACGGTTTCGCCGGCAGTCTCGATGATAGTATTTCTGCTGCAAGTACTTCTGATGTTCAAGATCGCC
TGTCAGCTCTTGAGTCACGAGTTCAGCAACAAGAAGATGAAATCACTGTGCTAAAGGCGGCTTTGGCTGA
TGTTTTGAGGCGTCTTGCAATCTCTGAAGATCATGTGGCCTCAGTGAAAAAATCAGTCTCAAGTAAAGGC
CAACCAAGCCCTCGAGCAGTTATTCCCATGTCCTGTATAACCAATGGAAGTGGTGCAAACAGAAAACCAA
GTCATACCAGTGCTGTCTCAATTGCAGGAAAAGAAACTCTTTCATCTGCTGCTAAAAGTGGTACAGAAAA
AAAGAAAGAAAAACCACAAGGACAGAGAGAAAAAAAAGAGGAATCTCATTCTAATGATCAAAGTCCACAA
ATTCGAGCATCACCTTCTCCCCAGCCCTCTTCACAACCTCTCCAAATACACAGACAAACTCCAGAAAGCA
AGAATGCTACTCCCACCAAAAGCATAAAACGACCATCACCAGCTGAAAAGTCACATAATTCTTGGGAAAA
TTCAGATGATAGCCGTAATAAATTGTCGAAAATACCTTCAACACCCAAATTAATACCAAAAGTTACCAAA

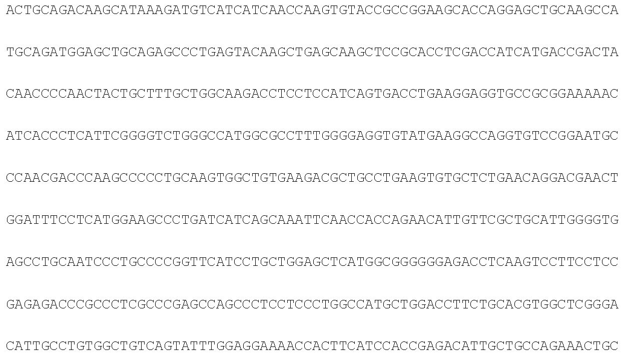

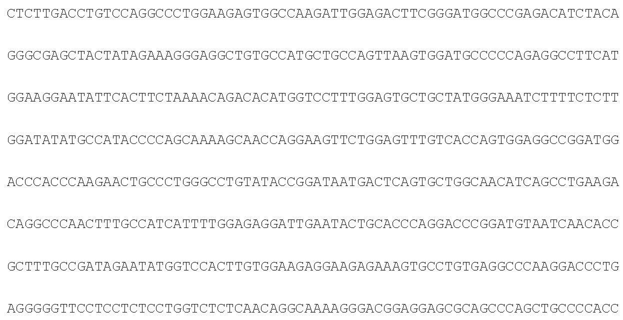

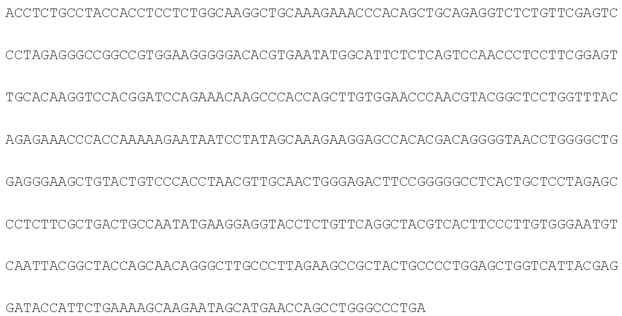

EML4-ALK variant 3b (2391nt)
EML4 exons 1-6 + cryptic exon(33 nt) + ALK exons 20-30
(SEQ ID NO: 4)
ATGGACGGTTTCGCCGGCAGTCTCGATGATAGTATTTCTGCTGCAAGTACTTCTGATGTTCAAGATCGCC
TGTCAGCTCTTGAGTCACGAGTTCAGCAACAAGAAGATGAAATCACTGTGCTAAAGGCGGCTTTGGCTGA
TGTTTTGAGGCGTCTTGCAATCTCTGAAGATCATGTGGCCTCAGTGAAAAAATCAGTCTCAAGTAAAGGC
CAACCAAGCCCTCGAGCAGTTATTCCCATGTCCTGTATAACCAATGGAAGTGGTGCAAACAGAAAACCAA
GTCATACCAGTGCTGTCTCAATTGCAGGAAAAGAAACTCTTTCATCTGCTGCTAAAAGTGGTACAGAAAA
AAAGAAAGAAAAACCACAAGGACAGAGAGAAAAAAAAGAGGAATCTCATTCTAATGATCAAAGTCCACAA
ATTCGAGCATCACCTTCTCCCCAGCCCTCTTCACAACCTCTCCAAATACACAGACAAACTCCAGAAAGCA
AGAATGCTACTCCCACCAAAAGCATAAAACGACCATCACCAGCTGAAAAGTCACATAATTCTTGGGAAAA
TTCAGATGATAGCCGTAATAAATTGTCGAAAATACCTTCAACACCCAAATTAATACCAAAAGTTACCAAA
ACTGCAGACAAGCATAAAGATGTCATCATCAACCAAGcaaaaatgtcaactcgcgaaaaaaacagccaag

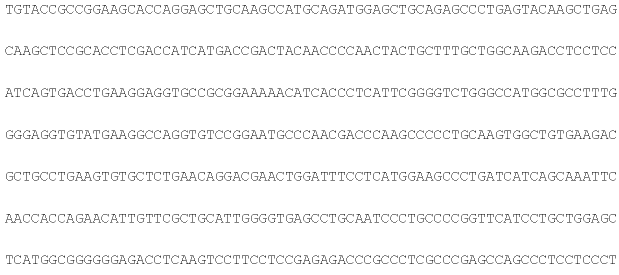

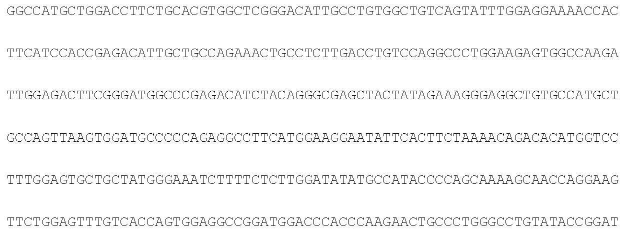

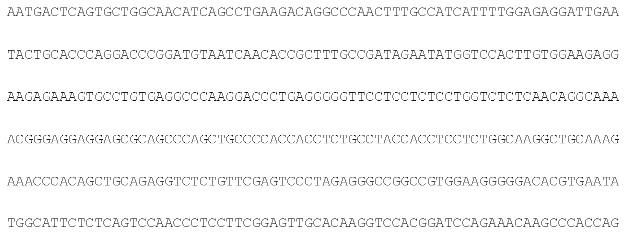

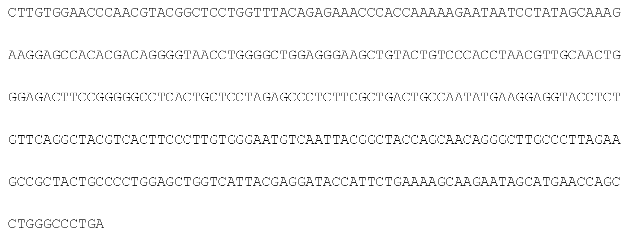

EML4-ALK variant 4 (3294 nt)
EML4 exons 1-14 + unknown 11 nt + ALK exons 20 (−49nt) −30
(SEQ ID NO: 5)
ATGGACGGTTTCGCCGGCAGTCTCGATGATAGTATTTCTGCTGCAAGTACTTCTGATGTTCAAGATCGCC
TGTCAGCTCTTGAGTCACGAGTTCAGCAACAAGAAGATGAAATCACTGTGCTAAAGGCGGCTTTGGCTGA
TGTTTTGAGGCGTCTTGCAATCTCTGAAGATCATGTGGCCTCAGTGAAAAAATCAGTCTCAAGTAAAGGC
CAACCAAGCCCTCGAGCAGTTATTCCCATGTCCTGTATAACCAATGGAAGTGGTGCAAACAGAAAACCAA
GTCATACCAGTGCTGTCTCAATTGCAGGAAAAGAAACTCTTTCATCTGCTGCTAAAAGTGGTACAGAAAA
AAAGAAAGAAAAACCACAAGGACAGAGAGAAAAAAAAGAGGAATCTCATTCTAATGATCAAAGTCCACAA
ATTCGAGCATCACCTTCTCCCCAGCCCTCTTCACAACCTCTCCAAATACACAGACAAACTCCAGAAAGCA
AGAATGCTACTCCCACCAAAAGCATAAAACGACCATCACCAGCTGAAAAGTCACATAATTCTTGGGAAAA
TTCAGATGATAGCCGTAATAAATTGTCGAAAATACCTTCAACACCCAAATTAATACCAAAAGTTACCAAA
ACTGCAGACAAGCATAAAGATGTCATCATCAACCAAGAAGGAGAATATATTAAAATGTTTATGCGCGGTC
GGCCAATTACCATGTTCATTCCTTCCGATGTTGACAACTATGATGACATCAGAACGGAACTGCCTCCTGA
GAAGCTCAAACTGGAGTGGGCATATGGTTATCGAGGAAAGGACTGTAGAGCTAATGTTTACCTTCTTCCG
ACCGGGGAAATAGTTTATTTCATTGCATCAGTAGTAGTACTATTTAATTATGAGGAGAGAACTCAGCGAC
ACTACCTGGGCCATACAGACTGTGTGAAATGCCTTGCTATACATCCTGACAAAATTAGGATTGCAACTGG
ACAGATAGCTGGCGTGGATAAAGATGGAAGGCCTCTACAACCCCACGTCAGAGTGTGGGATTCTGTTACT
CTATCCACACTGCAGATTATTGGACTTGGCACTTTTGAGCGTGGAGTAGGATGCCTGGATTTTTCAAAAG
CAGATTCAGGTGTTCATTTATGTGTTATTGATGACTCCAATGAGCATATGCTTACTGTATGGGACTGGCA
GAAGAAAGCAAAAGGAGCAGAAATAAAGACAACAAATGAAGTTGTTTTGGCTGTGGAGTTTCACCCAACA
GATGCAAATACCATAATTACATGCGGTAAATCTCATATTTTCTTCTGGACCTGGAGCGGCAATTCACTAA
CAAGAAAACAGGGAATTTTTGGGAAATATGAAAAGCCAAAATTTGTGCAGTGTTTAGCATTCTTGGGGAA
TGGAGATGTTCTTACTGGAGACTCAGGTGGAGTCATGCTTATATGGAGCAAAACTACTGTAGAGCCCACA
CCTGGGAAAGGACCTAAAGGTGTATATCAAATCAGCAAACAAATCAAAGCTCATGATGGCAGTGTGTTCA
CACTTTGTCAGATGAGAAATGGGATGTTATTAACTGGAGGAGGGAAAGACAGAAAAATAATTCTGTGGGA

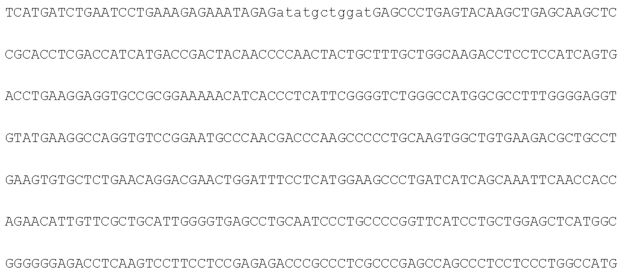

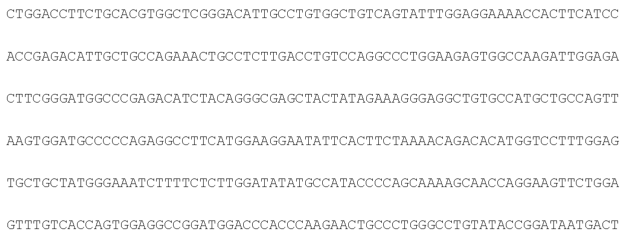

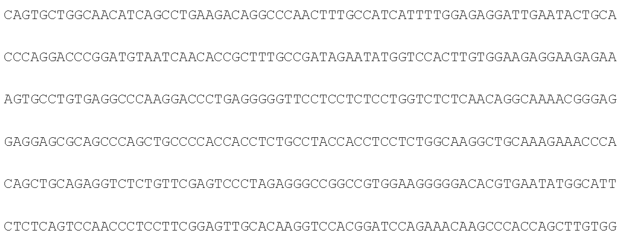

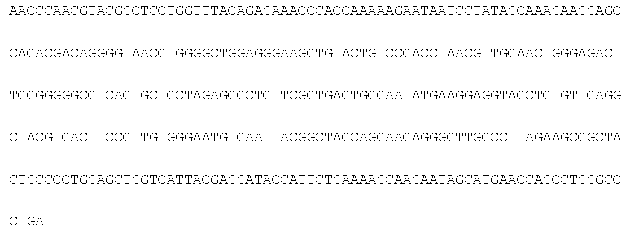

EML4-ALK variant 5a (1899 nt)
EML4 exons 1-2 + ALK exons 20-30
(SEQ ID NO: 6)
ATGGACGGTTTCGCCGGCAGTCTCGATGATAGTATTTCTGCTGCAAGTACTTCTGATGTTCAAGATCGCC
TGTCAGCTCTTGAGTCACGAGTTCAGCAACAAGAAGATGAAATCACTGTGCTAAAGGCGGCTTTGGCTGA

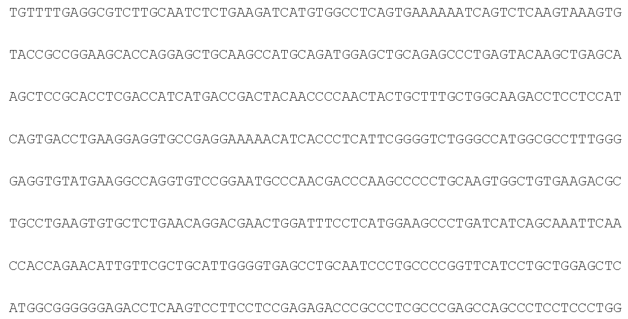

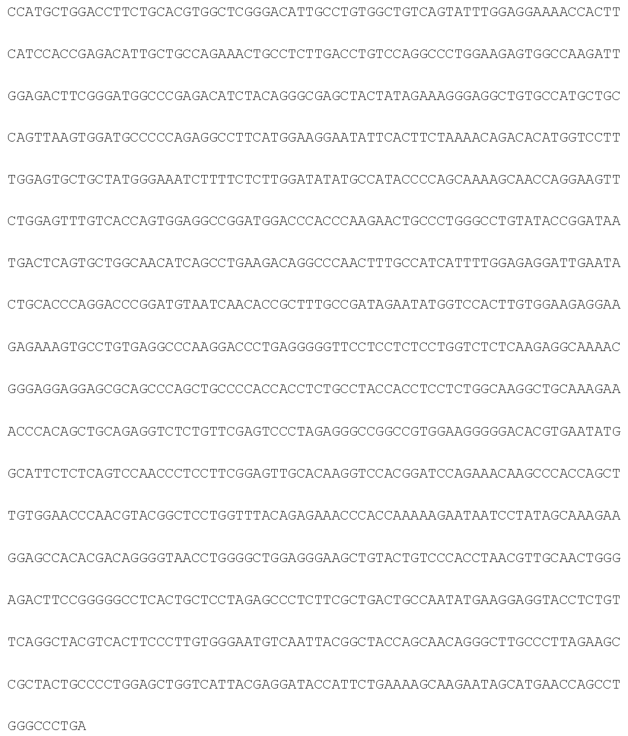

EML4-ALK variant 5b (2016 nt)
EML4 exons 1-2 + unknown 117 nt + ALK exons 20-30
(SEQ ID NO: 7)
ATGGACGGTTTCGCCGGCAGTCTCGATGATAGTATTTCTGCTGCAAGTACTTCTGATGTTCAAGATCGCC
TGTCAGCTCTTGAGTCACGAGTTCAGCAACAAGAAGATGAAATCACTGTGCTAAAGGCGGCTTTGGCTGA
TGTTTTGAGGCGTCTTGCAATCTCTGAAGATCATGTGGCCTCAGTGAAAAAATCAGTCTCAAGTAAAGgt
tcagagctcaggggaggatatggagatccagggaggcttcctgtaggaagtggcctgtgtagtgcttcaa

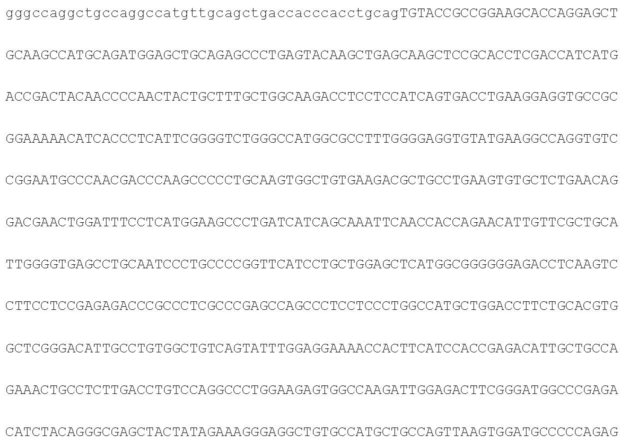

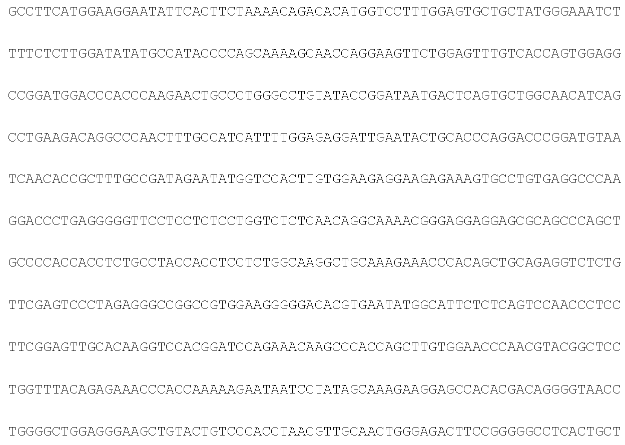

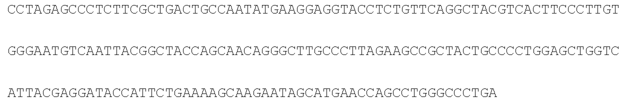

EML4-ALK variant 6 (3747 nt)
EML4 exons 1-18 + ALK exons 20-30
(SEQ ID NO:8)
ATGGACGGTTTCGCCGGCAGTCTCGATGATAGTATTTCTGCTGCAAGTACTTCTGATGTTCAAGATCGCC
TGTCAGCTCTTGAGTCACGAGTTCAGCAACAAGAAGATGAAATCACTGTGCTAAAGGCGGCTTTGGCTGA
CAACCAAGCCCTCGAGCAGTTATTCCCATGTCCTGTATAACCAATGGAAGTGGTGCAAACAGAAAACCAA
GTCATACCAGTGCTGTCTCAATTGCAGGAAAAGAAACTCTTTCATCTGCTGCTAAAAGTGGTACAGAAAA
AAAGAAAGAAAAACCACAAGGACAGAGAGAAAAAAAAGAGGAATCTCATTCTAATGATCAAAGTCCACAA
ATTCGAGCATCACCTTCTCCCCAGCCCTCTTCACAACCTCTCCAAATACACAGACAAACTCCAGAAAGCA
TTCAGATGATAGCCGTAATAAATTGTCGAAAATACCTTCAACACCCAAATTAATACCAAAAGTTACCAAA
ACTGCAGACAAGCATAAAGATGTCATCATCAACCAAGAAGGAGAATATATTAAAATGTTTATGCGCGGTC
GGCCAATTACCATGTTCATTCCTTCCGATGTTGACAACTATGATGACATCAGAACGGAACTGCCTCCTGA
GAAGCTCAAACTGGAGTGGGCATATGGTTATCGAGGAAAGGACTGTAGAGCTAATGTTTACCTTCTTCCG
ACCGGGGAAATAGTTTATTTCATTGCATCAGTAGTAGTACTATTTAATTATGAGGAGAGAACTCAGCGAC
ACTACCTGGGCCATACAGACTGTGTGAAATGCCTTGCTATACATCCTGACAAAATTAGGATTGCAACTGG
ACAGATAGCTGGCGTGGATAAAGATGGAAGGCCTCTACAACCCCACGTCAGAGTGTGGGATTCTGTTACT
CTATCCACACTGCAGATTATTGGACTTGGCACTTTTGAGCGTGGAGTAGGATGCCTGGATTTTTCAAAAG
CAGATTCAGGTGTTCATTTATGTGTTATTGATGACTCCAATGAGCATATGCTTACTGTATGGGACTGGCA
GAAGAAAGCAAAAGGAGCAGAAATAAAGACAACAAATGAAGTTGTTTTGGCTGTGGAGTTTCACCCAACA
GATGCAAATACCATAATTACATGCGGTAAATCTCATATTTTCTTCTGGACCTGGAGCGGCAATTCACTAA
CAAGAAAACAGGGAATTTTTGGGAAATATGAAAAGCCAAAATTTGTGCAGTGTTTAGCATTCTTGGGGAA
TGGAGATGTTCTTACTGGAGACTCAGGTGGAGTCATGCTTATATGGAGCAAAACTACTGTAGAGCCCACA
CCTGGGAAAGGACCTAAAGGTGTATATCAAATCAGCAAACAAATCAAAGCTCATGATGGCAGTGTGTTCA
TCATGATCTGAATCCTGAAAGAGAAATAGAGGTTCCTGATCAGTATGGCACAATCAGAGCTGTAGCAGAA
GGAAAGGCAGATCAATTTTTAGTAGGCACATCACGAAACTTTATTTTACGAGGAACATTTAATGATGGCT
TCCAAATAGAAGTACAGGGTCATACAGATGAGCTTTGGGGTCTTGCCACACATCCCTTCAAAGATTTGCT
CTTGACATGTGCTCAGGACAGGCAGGTGTGCCTGTGGAACTCAATGGAACACAGGCTGGAATGGACCAGG
CTGGTAGATGAACCAGGACACTGTGCAGATTTTCATCCAAGTGGCACAGTGGTGGCCATAGGAACGCACT
CAGGCAGGTGGTTTGTTCTGGATGCAGAAACCAGAGATCTAGTTTCTATCCACACAGACGGGAATGAACA

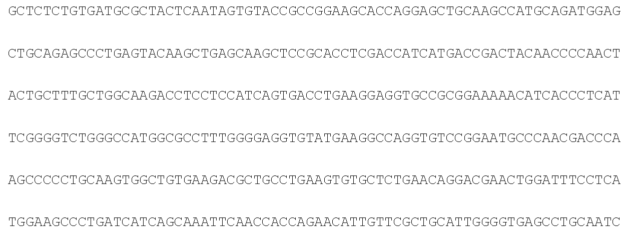

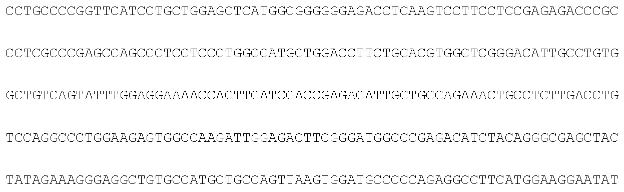

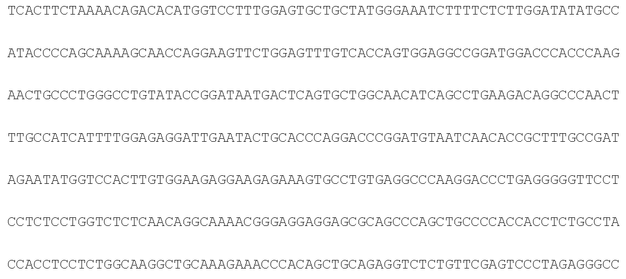

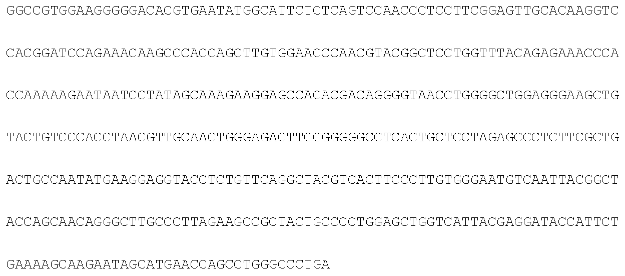

In SEQ ID NOs: 1-8, upper case, non-highlighted sequence is EML4 sequence, highlighted sequence is ALK sequence, and lower case sequence is cryptic or intronic sequence.

TFG-ALK (2614 nt; GenBank Accession No. AF125093)
(SEQ ID NO: 9)
CCTCCGCAAGCCGTCTTTCTCTAGAGTTGTATATATAGAACATCCTGGAGTCCACCATGAACGGACAGTT
GGATCTAAGTGGGAAGCTAATCATCAAAGCTCAACTTGGGGAGGATATTCGGCGAATTCCTATTCATAAT
GAAGATATTACTTATGATGAATTAGTGCTAATGATGCAACGAGTTTTCAGAGGAAAACTTCTGAGTAATG
ATGAAGTAACAATAAAGTATAAAGATGAAGATGGAGATCTTATAACAATTTTTGATAGTTCTGACCTTTC
CTTTGCAATTCAGTGCAGTAGGATACTGAAACTGACATTATTTGTTAATGGCCAGCCAAGACCCCTTGAA
TCAAGTCAGGTGAAATATCTCCGTCGAGAACTGATAGAACTTCGAAATAAAGTGAATCGTTTATTGGATA
GCTTGGAACCACCTGGAGAACCAGGACCTTCCACCAATATTCCTGAAAATGTGTACCGCCGGAAGCACCA
GGAGCTGCAAGCCATGCAGATGGAGCTGCAGAGCCCTGAGTACAAGCTGAGCAAGCTCCGCACCTCGACC
ATCATGACCGACTACAACCCCAACTACTGCTTTGCTGGCAAGACCTCCTCCATCAGTGACCTGAAGGAGG
TGCCGCGGAAAAACATCACCCTCATTCGGGGTCTGGGCCATGGCGCCTTTGGGGAGGTGTATGAAGGCCA
GGTGTCCGGAATGCCCAACGACCCAAGCCCCCTGCAAGTGGCTGTGAAGACGCTGCCTGAAGTGTGCTCT
GAACAGGACGAACTGGATTTCCTCATGGAAGCCCTGATCATCAGCAAATTCAACCACCAGAACATTGTTC
GCTGCATTGGGGTGAGCCTGCAATCCCTGCCCCGGTTCATCCTGCTGGAGCTCATGGCGGGGGGAGACCT
CAAGTCCTTCCTCCGAGAGACCCGCCCTCGCCCGAGCCAGCCCTCCTCCCTGGCCATGCTGGACCTTCTG
CACGTGGCTCGGGACATTGCCTGTGGCTGTCAGTATTTGGAGGAAAACCACTTCATCCACCGAGACATTG
CTGCCAGAAACTGCCTCTTGACCTGTCCAGGCCCTGGAAGAGTGGCCAAGATTGGAGACTTCGGGATGGC
CCGAGACATCTACAGGGCGAGCTACTATAGAAAGGGAGGCTGTGCCATGCTGCCAGTTAAGTGGATGCCC
CCAGAGGCCTTCATGGAAGGAATATTCACTTCTAAAACAGACACATGGTCCTTTGGAGTGCTGCTATGGG
AAATCTTTTCTCTTGGATATATGCCATACCCCAGCAAAAGCAACCAGGAAGTTCTGGAGTTTGTCACCAG
TGGAGGCCGGATGGACCCACCCAAGAACTGCCCTGGGCCTGTATACCGGATAATGACTCAGTGCTGGCAA
CATCAGCCTGAAGACAGGCCCAACTTTGCCATCATTTTGGAGAGGATTGAATACTGCACCCAGGACCCGG
ATGTAATCAACACCGCTTTGCCGATAGAATATGGTCCACTTGTGGAAGAGGAAGAGAAAGTGCCTGTGAG
GCCCAAGGACCCTGAGGGGGTTCCTCCTCTCCTGGTCTCTCAACAGGCAAAACGGGAGGAGGAGCGCAGC
CCAGCTGCCCCACCACCTCTGCCTACCACCTCCTCTGGCAAGGCTGCAAAGAAACCCACAGCTGCAGAGG
TCTCTGTTCGAGTCCCTAGAGGGCCGGCCGTGGAAGGGGGACACGTGAATATGGCATTCTCTCAGTCCAA
CCCTCCTTCGGAGTTGCACAAGGTCCACGGATCCAGAAACAAGCCCACCAGCTTGTGGAACCCAACGTAC
GGCTCCTGGTTTACAGAGAAACCCACCAAAAAGAATAATCCTATAGCAAAGAAGGAGCCACACGACAGGG
GTAACCTGGGGCTGGAGGGAAGCTGTACTGTCCCACCTAACGTTGCAACTGGGAGACTTCCGGGGGCCTC
ACTGCTCCTAGAGCCCTCTTCGCTGACTGCCAATATGAAGGAGGTACCTCTGTTCAGGCTACGTCACTTC
CCTTGTGGGAATGTCAATTACGGCTACCAGCAACAGGGCTTGCCCTTAGAAGCCGCTACTGCCCCTGGAG
CTGGTCATTACGAGGATACCATTCTGAAAAGCAAGAATAGCATGAACCAGCCTGGGCCCTGAGCTCGGTC
GCACACTCACTTCTCTTCCTTGGGATCCCTAAGACCGTGGAGGAGAGAGAGGCAATGGCTCCTTCACAAA
CCAGAGACCAAATGTCACGTTTTGTTTTGTGCCAACCTATTTTGAAGTACCACCAAAAAAGCTGTATTTT
GAAAATGCTTTAGAAAGGTTTTGAGCATGGGTTCATCCTATTCTTTCGAAAGAAGAAAATATCATAAAAA
TGAGTGATAAATACAAGGCCCAGATGTGGTTGCATAAGGTTTTTATGCATGTTTGTTGTATACTTCCTTA
TGCTTCTTTTAAATTGTGTGTGCTCTGCTTCAATGTAGTCAGAATTAGCTGCTTCTATGTTTCATAGTTG
GGGTCATAGATGTTTCCTTGCCTTGTTGATGTGGACATGAGCCATTTGAGGGGAGAGGGAACGGAAATAA
AGGAGTTATTTGTAATGACTAAAA
KIF5B-ALK (4479 nt; GenBank Accession No. AB462413)
(SEQ ID NO: 10)
TGCGAGAAAGATGGCGGACCTGGCCGAGTGCAACATCAAAGTGATGTGTCGCTTCAGACCTCTCAACGAG
TCTGAAGTGAACCGCGGCGACAAGTACATCGCCAAGTTTCAGGGAGAAGACACGGTCGTGATCGCGTCCA
AGCCTTATGCATTTGATCGGGTGTTCCAGTCAAGCACATCTCAAGAGCAAGTGTATAATGACTGTGCAAA
GAAGATTGTTAAAGATGTACTTGAAGGATATAATGGAACAATATTTGCATATGGACAAACATCCTCTGGG
AAGACACACACAATGGAGGGTAAACTTCATGATCCAGAAGGCATGGGAATTATTCCAAGAATAGTGCAAG
ATATTTTTAATTATATTTACTCCATGGATGAAAATTTGGAATTTCATATTAAGGTTTCATATTTTGAAAT
ATATTTGGATAAGATAAGGGACCTGTTAGATGTTTCAAAGACCAACCTTTCAGTTCATGAAGACAAAAAC
CGAGTTCCCTATGTAAAGGGGTGCACAGAGCGTTTTGTATGTAGTCCAGATGAAGTTATGGATACCATAG
ATGAAGGAAAATCCAACAGACATGTAGCAGTTACAAATATGAATGAACATAGCTCTAGGAGTCACAGTAT
ATTTCTTATTAATGTCAAACAAGAGAACACACAAACGGAACAAAAGCTGAGTGGAAAACTTTATCTGGTT
GATTTAGCTGGTAGTGAAAAGGTTAGTAAAACTGGAGCTGAAGGTGCTGTGCTGGATGAAGCTAAAAACA
TCAACAAGTCACTTTCTGCTCTTGGAAATGTTATTTCTGCTTTGGCTGAGGGTAGTACATATGTTCCATA
TCGAGATAGTAAAATGACAAGAATCCTTCAAGATTCATTAGGTGGCAACTGTAGAACCACTATTGTAATT
TGCTGCTCTCCATCATCATACAATGAGTCTGAAACAAAATCTACACTCTTATTTGGCCAAAGGGCCAAAA
CAATTAAGAACACAGTTTGTGTCAATGTGGAGTTAACTGCAGAACAGTGGAAAAAGAAGTATGAAAAAGA
AAAAGAAAAAAATAAGATCCTGCGGAACACTATTCAGTGGCTTGAAAATGAGCTCAACAGATGGCGTAAT
GGGGAGACGGTGCCTATTGATGAACAGTTTGACAAAGAGAAAGCCAACTTGGAAGCTTTCACAGTGGATA
AAGATATTACTCTTACCAATGATAAACCAGCAACCGCAATTGGAGTTATAGGAAATTTTACTGATGCTGA
AAGAAGAAAGTGTGAAGAAGAAATTGCTAAATTATACAAACAGCTTGATGACAAGGATGAAGAAATTAAC
CAGCAAAGTCAACTGGTAGAGAAACTGAAGACGCAAATGTTGGATCAGGAGGAGCTTTTGGCATCTACCA
GAAGGGATCAAGACAATATGCAAGCTGAGCTGAATCGCCTTCAAGCAGAAAATGATGCCTCTAAAGAAGA
AGTGAAAGAAGTTTTACAGGCCCTAGAAGAACTTGCTGTCAATTATGATCAGAAGTCTCAGGAAGTTGAA
GACAAAACTAAGGAATATGAATTGCTTAGTGATGAATTGAATCAGAAATCGGCAACTTTAGCGAGTATAG
ATGCTGAGCTTCAGAAACTTAAGGAAATGACCAACCACCAGAAAAAACGAGCAGCTGAGATGATGGCATC
TTTACTAAAAGACCTTGCAGAAATAGGAATTGCTGTGGGAAATAATGATGTAAAGCAGCCTGAGGGAACT
GGCATGATAGATGAAGAGTTCACTGTTGCAAGACTCTACATTAGCAAAATGAAGTCAGAAGTAAAAACCA
TGGTGAAACGTTGCAAGCAGTTAGAAAGCACACAAACTGAGAGCAACAAAAAAATGGAAGAAAATGAAAA
GGAGTTAGCAGCATGTCAGCTTCGTATCTCTCAACATGAAGCCAAAATCAAGTCATTGACTGAATACCTT
CAAAATGTGGAACAAAAGAAAAGACAGTTGGAGGAATCTGTCGATGCCCTCAGTGAAGAACTAGTCCAGC
TTCGAGCACAAGAGAAAGTCCATGAAATGGAAAAGGAGCACTTAAATAAGGTTCAGACTGCAAATGAAGT
TAAGCAAGCTGTTGAACAGCAGATCCAGAGCCATAGAGAAACTCATCAAAAACAGATCAGTAGTTTGAGA
GATGAAGTAGAAGCAAAAGCAAAACTTATTACTGATCTTCAAGACCAAAACCAGAAAATGATGTTAGAGC
AGGAACGTCTAAGAGTAGAACATGAGAAGTTGAAAGCCACAGATCAGGAAAAGAGCAGAAAACTACATGA
ACTTACGGTTATGCAAGATAGACGAGAACAAGCAAGACAAGACTTGAAGGGTTTGGAAGAGACAGTGGCA
AAAGAACTTCAGACTTTACACAACCTGCGCAAACTCTTTGTTCAGGACCTGGCTACAAGAGTTAAAAAGA
GTGCTGAGATTGATTCTGATGACACCGGAGGCAGCGCTGCTCAGAAGCAAAAAATCTCCTTTCTTGAAAA
TAATCTTGAACAGCTCACTAAAGTGCACAAACAGTTGGTACGTGATAATGCAGATCTCCGCTGTGAACTT
CCTAAGTTGGAAAAGCGACTTCGAGCTACAGCTGAGAGAGTGAAAGCTTTGGAATCAGCACTGAAAGAAG
CTAAAGAAAATGCATCTCGTGATCGCAAACGCTATCAGCAAGAAGTAGATCGCATAAAGGAAGCAGTCAG
GTCAAAGAATATGGCCAGAAGAGGGCATTCTGCACAGATTGTGTACCGCCGGAAGCACCAGGAGCTGCAA
GCCATGCAGATGGAGCTGCAGAGCCCTGAGTACAAGCTGAGCAAGCTCCGCACCTCGACCATCATGACCG
ACTACAACCCCAACTACTGCTTTGCTGGCAAGACCTCCTCCATCAGTGACCTGAAGGAGGTGCCGCGGAA
AAACATCACCCTCATTCGGGGTCTGGGCCATGGCGCCTTTGGGGAGGTGTATGAAGGCCAGGTGTCCGGA
ATGCCCAACGACCCAAGCCCCCTGCAAGTGGCTGTGAAGACGCTGCCTGAAGTGTGCTCTGAACAGGACG
AACTGGATTTCCTCATGGAAGCCCTGATCATCAGCAAATTCAACCACCAGAACATTGTTCGCTGCATTGG
GGTGAGCCTGCAATCCCTGCCCCGGTTCATCCTGCTGGAGCTCATGGCGGGGGGAGACCTCAAGTCCTTC
CTCCGAGAGACCCGCCCTCGCCCGAGCCAGCCCTCCTCCCTGGCCATGCTGGACCTTCTGCACGTGGCTC
GGGACATTGCCTGTGGCTGTCAGTATTTGGAGGAAAACCACTTCATCCACCGAGACATTGCTGCCAGAAA
CTGCCTCTTGACCTGTCCAGGCCCTGGAAGAGTGGCCAAGATTGGAGACTTCGGGATGGCCCGAGACATC
TACAGGGCGAGCTACTATAGAAAGGGAGGCTGTGCCATGCTGCCAGTTAAGTGGATGCCCCCAGAGGCCT
TCATGGAAGGAATATTCACTTCTAAAACAGACACATGGTCCTTTGGAGTGCTGCTATGGGAAATCTTTTC
TCTTGGATATATGCCATACCCCAGCAAAAGCAACCAGGAAGTTCTGGAGTTTGTCACCAGTGGAGGCCGG
ATGGACCCACCCAAGAACTGCCCTGGGCCTGTATACCGGATAATGACTCAGTGCTGGCAACATCAGCCTG
AAGACAGGCCCAACTTTGCCATCATTTTGGAGAGGATTGAATACTGCACCCAGGACCCGGATGTAATCAA
CACCGCTTTGCCGATAGAATATGGTCCACTTGTGGAAGAGGAAGAGAAAGTGCCTGTGAGGCCCAAGGAC
CCTGAGGGGGTTCCTCCTCTCCTGGTCTCTCAACAGGCAAAACGGGAGGAGGAGCGCAGCCCAGCTGCCC
CACCACCTCTGCCTACCACCTCCTCTGGCAAGGCTGCAAAGAAACCCACAGCTGCAGAGGTCTCTGTTCG
AGTCCCTAGAGGGCCGGCCGTGGAAGGGGGACACGTGAATATGGCATTCTCTCAGTCCAACCCTCCTTCG
GAGTTGCACAAGGTCCACGGATCCAGAAACAAGCCCACCAGCTTGTGGAACCCAACGTACGGCTCCTGGT
TTACAGAGAAACCCACCAAAAAGAATAATCCTATAGCAAAGAAGGAGCCACACGACAGGGGTAACCTGGG
GCTGGAGGGAAGCTGTACTGTCCCACCTAACGTTGCAACTGGGAGACTTCCGGGGGCCTCACTGCTCCTA
GAGCCCTCTTCGCTGACTGCCAATATGAAGGAGGTACCTCTGTTCAGGCTACGTCACTTCCCTTGTGGGA
ATGTCAATTACGGCTACCAGCAACAGGGCTTGCCCTTAGAAGCCGCTACTGCCCCTGGAGCTGGTCATTA
CGAGGATACCATTCTGAAAAGCAAGAATAGCATGAACCAGCCTGGGCCCTGAGCTCGGTCGCACACTCA

The disclosed methods and arrays also include detecting wild-type (full-length) EML4 and ALK nucleic acids. Exemplary nucleic acid sequences of EML4 and ALK detected in at least some embodiments are as follows:

ALK (6267 nt; GenBank Accession No. NM_004304)
(SEQ ID NO: 11)
AGCTGCAAGTGGCGGGCGCCCAGGCAGATGCGATCCAGCGGCTCTGGGGGCGGCAGCGGTGGTAGCAGCT
GGTACCTCCCGCCGCCTCTGTTCGGAGGGTCGCGGGGCACCGAGGTGCTTTCCGGCCGCCCTCTGGTCGG
CCACCCAAAGCCGCGGGCGCTGATGATGGGTGAGGAGGGGGCGGCAAGATTTCGGGCGCCCCTGCCCTGA
ACGCCCTCAGCTGCTGCCGCCGGGGCCGCTCCAGTGCCTGCGAACTCTGAGGAGCCGAGGCGCCGGTGAG
AGCAAGGACGCTGCAAACTTGCGCAGCGCGGGGGCTGGGATTCACGCCCAGAAGTTCAGCAGGCAGACAG
TCCGAAGCCTTCCCGCAGCGGAGAGATAGCTTGAGGGTGCGCAAGACGGCAGCCTCCGCCCTCGGTTCCC
GCCCAGACCGGGCAGAAGAGCTTGGAGGAGCCAAAAGGAACGCAAAAGGCGGCCAGGACAGCGTGCAGCA
GCTGGGAGCCGCCGTTCTCAGCCTTAAAAGTTGCAGAGATTGGAGGCTGCCCCGAGAGGGGACAGACCCC
AGCTCCGACTGCGGGGGGCAGGAGAGGACGGTACCCAACTGCCACCTCCCTTCAACCATAGTAGTTCCTC
TGTACCGAGCGCAGCGAGCTACAGACGGGGGCGCGGCACTCGGCGCGGAGAGCGGGAGGCTCAAGGTCCC
AGCCAGTGAGCCCAGTGTGCTTGAGTGTCTCTGGACTCGCCCCTGAGCTTCCAGGTCTGTTTCATTTAGA
CTCCTGCTCGCCTCCGTGCAGTTGGGGGAAAGCAAGAGACTTGCGCGCACGCACAGTCCTCTGGAGATCA
GGTGGAAGGAGCCGCTGGGTACCAAGGACTGTTCAGAGCCTCTTCCCATCTCGGGGAGAGCGAAGGGTGA
GGCTGGGCCCGGAGAGCAGTGTAAACGGCCTCCTCCGGCGGGATGGGAGCCATCGGGCTCCTGTGGCTCC
TGCCGCTGCTGCTTTCCACGGCAGCTGTGGGCTCCGGGATGGGGACCGGCCAGCGCGCGGGCTCCCCAGC
TGCGGGGCCGCCGCTGCAGCCCCGGGAGCCACTCAGCTACTCGCGCCTGCAGAGGAAGAGTCTGGCAGTT
GACTTCGTGGTGCCCTCGCTCTTCCGTGTCTACGCCCGGGACCTACTGCTGCCACCATCCTCCTCGGAGC
TGAAGGCTGGCAGGCCCGAGGCCCGCGGCTCGCTAGCTCTGGACTGCGCCCCGCTGCTCAGGTTGCTGGG
GCCGGCGCCGGGGGTCTCCTGGACCGCCGGTTCACCAGCCCCGGCAGAGGCCCGGACGCTGTCCAGGGTG
CTGAAGGGCGGCTCCGTGCGCAAGCTCCGGCGTGCCAAGCAGTTGGTGCTGGAGCTGGGCGAGGAGGCGA
TCTTGGAGGGTTGCGTCGGGCCCCCCGGGGAGGCGGCTGTGGGGCTGCTCCAGTTCAATCTCAGCGAGCT
GTTCAGTTGGTGGATTCGCCAAGGCGAAGGGCGACTGAGGATCCGCCTGATGCCCGAGAAGAAGGCGTCG
GAAGTGGGCAGAGAGGGAAGGCTGTCCGCGGCAATTCGCGCCTCCCAGCCCCGCCTTCTCTTCCAGATCT
TCGGGACTGGTCATAGCTCCTTGGAATCACCAACAAACATGCCTTCTCCTTCTCCTGATTATTTTACATG
GAATCTCACCTGGATAATGAAAGACTCCTTCCCTTTCCTGTCTCATCGCAGCCGATATGGTCTGGAGTGC
AGCTTTGACTTCCCCTGTGAGCTGGAGTATTCCCCTCCACTGCATGACCTCAGGAACCAGAGCTGGTCCT
GGCGCCGCATCCCCTCCGAGGAGGCCTCCCAGATGGACTTGCTGGATGGGCCTGGGGCAGAGCGTTCTAA
GGAGATGCCCAGAGGCTCCTTTCTCCTTCTCAACACCTCAGCTGACTCCAAGCACACCATCCTGAGTCCG
TGGATGAGGAGCAGCAGTGAGCACTGCACACTGGCCGTCTCGGTGCACAGGCACCTGCAGCCCTCTGGAA
GGTACATTGCCCAGCTGCTGCCCCACAACGAGGCTGCAAGAGAGATCCTCCTGATGCCCACTCCAGGGAA
GCATGGTTGGACAGTGCTCCAGGGAAGAATCGGGCGTCCAGACAACCCATTTCGAGTGGCCCTGGAATAC
ATCTCCAGTGGAAACCGCAGCTTGTCTGCAGTGGACTTCTTTGCCCTGAAGAACTGCAGTGAAGGAACAT
CCCCAGGCTCCAAGATGGCCCTGCAGAGCTCCTTCACTTGTTGGAATGGGACAGTCCTCCAGCTTGGGCA
GGCCTGTGACTTCCACCAGGACTGTGCCCAGGGAGAAGATGAGAGCCAGATGTGCCGGAAACTGCCTGTG
GGTTTTTACTGCAACTTTGAAGATGGCTTCTGTGGCTGGACCCAAGGCACACTGTCACCCCACACTCCTC
AATGGCAGGTCAGGACCCTAAAGGATGCCCGGTTCCAGGACCACCAAGACCATGCTCTATTGCTCAGTAC
CACTGATGTCCCCGCTTCTGAAAGTGCTACAGTGACCAGTGCTACGTTTCCTGCACCGATCAAGAGCTCT
CCATGTGAGCTCCGAATGTCCTGGCTCATTCGTGGAGTCTTGAGGGGAAACGTGTCCTTGGTGCTAGTGG
AGAACAAAACCGGGAAGGAGCAAGGCAGGATGGTCTGGCATGTCGCCGCCTATGAAGGCTTGAGCCTGTG
GCAGTGGATGGTGTTGCCTCTCCTCGATGTGTCTGACAGGTTCTGGCTGCAGATGGTCGCATGGTGGGGA
CAAGGATCCAGAGCCATCGTGGCTTTTGACAATATCTCCATCAGCCTGGACTGCTACCTCACCATTAGCG
GAGAGGACAAGATCCTGCAGAATACAGCACCCAAATCAAGAAACCTGTTTGAGAGAAACCCAAACAAGGA
GCTGAAACCCGGGGAAAATTCACCAAGACAGACCCCCATCTTTGACCCTACAGTTCATTGGCTGTTCACC
ACATGTGGGGCCAGCGGGCCCCATGGCCCCACCCAGGCACAGTGCAACAACGCCTACCAGAACTCCAACC
TGAGCGTGGAGGTGGGGAGCGAGGGCCCCCTGAAAGGCATCCAGATCTGGAAGGTGCCAGCCACCGACAC
CTACAGCATCTCGGGCTACGGAGCTGCTGGCGGGAAAGGCGGGAAGAACACCATGATGCGGTCCCACGGC
GTGTCTGTGCTGGGCATCTTCAACCTGGAGAAGGATGACATGCTGTACATCCTGGTTGGGCAGCAGGGAG
AGGACGCCTGCCCCAGTACAAACCAGTTAATCCAGAAAGTCTGCATTGGAGAGAACAATGTGATAGAAGA
AGAAATCCGTGTGAACAGAAGCGTGCATGAGTGGGCAGGAGGCGGAGGAGGAGGGGGTGGAGCCACCTAC
GTATTTAAGATGAAGGATGGAGTGCCGGTGCCCCTGATCATTGCAGCCGGAGGTGGTGGCAGGGCCTACG
GGGCCAAGACAGACACGTTCCACCCAGAGAGACTGGAGAATAACTCCTCGGTTCTAGGGCTAAACGGCAA
TTCCGGAGCCGCAGGTGGTGGAGGTGGCTGGAATGATAACACTTCCTTGCTCTGGGCCGGAAAATCTTTG
CAGGAGGGTGCCACCGGAGGACATTCCTGCCCCCAGGCCATGAAGAAGTGGGGGTGGGAGACAAGAGGGG
GTTTCGGAGGGGGTGGAGGGGGGTGCTCCTCAGGTGGAGGAGGCGGAGGATATATAGGCGGCAATGCAGC
CTCAAACAATGACCCCGAAATGGATGGGGAAGATGGGGTTTCCTTCATCAGTCCACTGGGCATCCTGTAC
ACCCCAGCTTTAAAAGTGATGGAAGGCCACGGGGAAGTGAATATTAAGCATTATCTAAACTGCAGTCACT
GTGAGGTAGACGAATGTCACATGGACCCTGAAAGCCACAAGGTCATCTGCTTCTGTGACCACGGGACGGT
GCTGGCTGAGGATGGCGTCTCCTGCATTGTGTCACCCACCCCGGAGCCACACCTGCCACTCTCGCTGATC
CTCTCTGTGGTGACCTCTGCCCTCGTGGCCGCCCTGGTCCTGGCTTTCTCCGGCATCATGATTGTGTACC
GCCGGAAGCACCAGGAGCTGCAAGCCATGCAGATGGAGCTGCAGAGCCCTGAGTACAAGCTGAGCAAGCT
CCGCACCTCGACCATCATGACCGACTACAACCCCAACTACTGCTTTGCTGGCAAGACCTCCTCCATCAGT
GACCTGAAGGAGGTGCCGCGGAAAAACATCACCCTCATTCGGGGTCTGGGCCATGGCGCCTTTGGGGAGG
TGTATGAAGGCCAGGTGTCCGGAATGCCCAACGACCCAAGCCCCCTGCAAGTGGCTGTGAAGACGCTGCC
TGAAGTGTGCTCTGAACAGGACGAACTGGATTTCCTCATGGAAGCCCTGATCATCAGCAAATTCAACCAC
CAGAACATTGTTCGCTGCATTGGGGTGAGCCTGCAATCCCTGCCCCGGTTCATCCTGCTGGAGCTCATGG
CGGGGGGAGACCTCAAGTCCTTCCTCCGAGAGACCCGCCCTCGCCCGAGCCAGCCCTCCTCCCTGGCCAT
GCTGGACCTTCTGCACGTGGCTCGGGACATTGCCTGTGGCTGTCAGTATTTGGAGGAAAACCACTTCATC
CACCGAGACATTGCTGCCAGAAACTGCCTCTTGACCTGTCCAGGCCCTGGAAGAGTGGCCAAGATTGGAG
ACTTCGGGATGGCCCGAGACATCTACAGGGCGAGCTACTATAGAAAGGGAGGCTGTGCCATGCTGCCAGT
TAAGTGGATGCCCCCAGAGGCCTTCATGGAAGGAATATTCACTTCTAAAACAGACACATGGTCCTTTGGA
GTGCTGCTATGGGAAATCTTTTCTCTTGGATATATGCCATACCCCAGCAAAAGCAACCAGGAAGTTCTGG
AGTTTGTCACCAGTGGAGGCCGGATGGACCCACCCAAGAACTGCCCTGGGCCTGTATACCGGATAATGAC
TCAGTGCTGGCAACATCAGCCTGAAGACAGGCCCAACTTTGCCATCATTTTGGAGAGGATTGAATACTGC
ACCCAGGACCCGGATGTAATCAACACCGCTTTGCCGATAGAATATGGTCCACTTGTGGAAGAGGAAGAGA
AAGTGCCTGTGAGGCCCAAGGACCCTGAGGGGGTTCCTCCTCTCCTGGTCTCTCAACAGGCAAAACGGGA
GGAGGAGCGCAGCCCAGCTGCCCCACCACCTCTGCCTACCACCTCCTCTGGCAAGGCTGCAAAGAAACCC
ACAGCTGCAGAGATCTCTGTTCGAGTCCCTAGAGGGCCGGCCGTGGAAGGGGGACACGTGAATATGGCAT
TCTCTCAGTCCAACCCTCCTTCGGAGTTGCACAAGGTCCACGGATCCAGAAACAAGCCCACCAGCTTGTG
GAACCCAACGTACGGCTCCTGGTTTACAGAGAAACCCACCAAAAAGAATAATCCTATAGCAAAGAAGGAG
CCACACGACAGGGGTAACCTGGGGCTGGAGGGAAGCTGTACTGTCCCACCTAACGTTGCAACTGGGAGAC
TTCCGGGGGCCTCACTGCTCCTAGAGCCCTCTTCGCTGACTGCCAATATGAAGGAGGTACCTCTGTTCAG
GCTACGTCACTTCCCTTGTGGGAATGTCAATTACGGCTACCAGCAACAGGGCTTGCCCTTAGAAGCCGCT
ACTGCCCCTGGAGCTGGTCATTACGAGGATACCATTCTGAAAAGCAAGAATAGCATGAACCAGCCTGGGC
CCTGAGCTCGGTCGCACACTCACTTCTCTTCCTTGGGATCCCTAAGACCGTGGAGGAGAGAGAGGCAATG
GCTCCTTCACAAACCAGAGACCAAATGTCACGTTTTGTTTTGTGCCAACCTATTTTGAAGTACCACCAAA
AAAGCTGTATTTTGAAAATGCTTTAGAAAGGTTTTGAGCATGGGTTCATCCTATTCTTTCGAAAGAAGAA
AATATCATAAAAATGAGTGATAAATACAAGGCCCAGATGTGGTTGCATAAGGTTTTTATGCATGTTTGTT
GTATACTTCCTTATGCTTCTTTCAAATTGTGTGTGCTCTGCTTCAATGTAGTCAGAATTAGCTGCTTCTA
TGTTTCATAGTTGGGGTCATAGATGTTTCCTTGCCTTGTTGATGTGGACATGAGCCATTTGAGGGGAGAG
GGAACGGAAATAAAGGAGTTATTTGTAATGACTAAAA
EML4 (5565 nt; GenBank Accession No. NM_019063)
(SEQ ID NO: 12)
GGCGCGGCGCTCGCGGCTGCTGCCTGGGAGGGAGGCCGGGCAGGCGGCTGAGCGGCGCGGCTCTCAACGT
GACGGGGAAGTGGTTCGGGCGGCCGCGGCTTACTACCCCAGGGCGAACGGACGGACGACGGAGGCGGGAG
CCGGTAGCCGAGCCGGGCGACCTAGAGAACGAGCGGGTCAGGCTCAGCGTCGGCCACTCTGTCGGTCCGC
TGAATGAAGTGCCCGCCCCTCTAAGCCCGGAGCCCGGCGCTTTCCCCGCAAGATGGACGGTTTCGCCGGC
AGTCTCGATGATAGTATTTCTGCTGCAAGTACTTCTGATGTTCAAGATCGCCTGTCAGCTCTTGAGTCAC
GAGTTCAGCAACAAGAAGATGAAATCACTGTGCTAAAGGCGGCTTTGGCTGATGTTTTGAGGCGTCTTGC
AATCTCTGAAGATCATGTGGCCTCAGTGAAAAAATCAGTCTCAAGTAAAGGCCAACCAAGCCCTCGAGCA
GTTATTCCCATGTCCTGTATAACCAATGGAAGTGGTGCAAACAGAAAACCAAGTCATACCAGTGCTGTCT
CAATTGCAGGAAAAGAAACTCTTTCATCTGCTGCTAAAAGTGGTACAGAAAAAAAGAAAGAAAAACCACA
AGGACAGAGAGAAAAAAAAGAGGAATCTCATTCTAATGATCAAAGTCCACAAATTCGAGCATCACCTTCT
CCCCAGCCCTCTTCACAACCTCTCCAAATACACAGACAAACTCCAGAAAGCAAGAATGCTACTCCCACCA
AAAGCATAAAACGACCATCACCAGCTGAAAAGTCACATAATTCTTGGGAAAATTCAGATGATAGCCGTAA
TAAATTGTCGAAAATACCTTCAACACCCAAATTAATACCAAAAGTTACCAAAACTGCAGACAAGCATAAA
GATGTCATCATCAACCAAGAAGGAGAATATATTAAAATGTTTATGCGCGGTCGGCCAATTACCATGTTCA
TTCCTTCCGATGTTGACAACTATGATGACATCAGAACGGAACTGCCTCCTGAGAAGCTCAAACTGGAGTG
GGCATATGGTTATCGAGGAAAGGACTGTAGAGCTAATGTTTACCTTCTTCCGACCGGGGAAATAGTTTAT
TTCATTGCATCAGTAGTAGTACTATTTAATTATGAGGAGAGAACTCAGCGACACTACCTGGGCCATACAG
ACTGTGTGAAATGCCTTGCTATACATCCTGACAAAATTAGGATTGCAACTGGACAGATAGCTGGCGTGGA
TAAAGATGGAAGGCCTCTACAACCCCACGTCAGAGTGTGGGATTCTGTTACTCTATCCACACTGCAGATT
ATTGGACTTGGCACTTTTGAGCGTGGAGTAGGATGCCTGGATTTTTCAAAAGCAGATTCAGGTGTTCATT
TATGTGTTATTGATGACTCCAATGAGCATATGCTTACTGTATGGGACTGGCAGAAGAAAGCAAAAGGAGC
AGAAATAAAGACAACAAATGAAGTTGTTTTGGCTGTGGAGTTTCACCCAACAGATGCAAATACCATAATT
ACATGCGGTAAATCTCATATTTTCTTCTGGACCTGGAGCGGCAATTCACTAACAAGAAAACAGGGAATTT
TTGGGAAATATGAAAAGCCAAAATTTGTGCAGTGTTTAGCATTCTTGGGGAATGGAGATGTTCTTACTGG
AGACTCAGGTGGAGTCATGCTTATATGGAGCAAAACTACTGTAGAGCCCACACCTGGGAAAGGACCTAAA
GGTGTATATCAAATCAGCAAACAAATCAAAGCTCATGATGGCAGTGTGTTCACACTTTGTCAGATGAGAA
ATGGGATGTTATTAACTGGAGGAGGGAAAGACAGAAAAATAATTCTGTGGGATCATGATCTGAATCCTGA
AAGAGAAATAGAGGTTCCTGATCAGTATGGCACAATCAGAGCTGTAGCAGAAGGAAAGGCAGATCAATTT
TTAGTAGGCACATCACGAAACTTTATTTTACGAGGAACATTTAATGATGGCTTCCAAATAGAAGTACAGG
GTCATACAGATGAGCTTTGGGGTCTTGCCACACATCCCTTCAAAGATTTGCTCTTGACATGTGCTCAGGA
CAGGCAGGTGTGCCTGTGGAACTCAATGGAACACAGGCTGGAATGGACCAGGCTGGTAGATGAACCAGGA
CACTGTGCAGATTTTCATCCAAGTGGCACAGTGGTGGCCATAGGAACGCACTCAGGCAGGTGGTTTGTTC
TGGATGCAGAAACCAGAGATCTAGTTTCTATCCACACAGACGGGAATGAACAGCTCTCTGTGATGCGCTA
CTCAATAGATGGTACCTTCCTGGCTGTAGGATCTCATGACAACTTTATTTACCTCTATGTAGTCTCTGAA
AATGGAAGAAAATATAGCAGATATGGAAGGTGCACTGGACATTCCAGCTACATCACACACCTTGACTGGT
CCCCAGACAACAAGTATATAATGTCTAACTCGGGAGACTATGAAATATTGTACTGGGACATTCCAAATGG
CTGCAAACTAATCAGGAATCGATCGGATTGTAAGGACATTGATTGGACGACATATACCTGTGTGCTAGGA
TTTCAAGTATTTGGTGTCTGGCCAGAAGGATCTGATGGGACAGATATCAATGCACTGGTGCGATCCCACA
ATAGAAAGGTGATAGCTGTTGCCGATGACTTTTGTAAAGTCCATCTGTTTCAGTATCCCTGCTCCAAAGC
AAAGGCTCCCAGTCACAAGTACAGTGCCCACAGCAGCCATGTCACCAATGTCAGTTTTACTCACAATGAC
AGTCACCTGATATCAACTGGTGGAAAAGACATGAGCATCATTCAGTGGAAACTTGTGGAAAAGTTATCTT
TGCCTCAGAATGAGACTGTAGCGGATACTACTCTAACCAAAGCCCCCGTCTCTTCCACTGAAAGTGTCAT
CCAATCTAATACTCCCACACCGCCTCCTTCTCAGCCCTTAAATGAGACAGCTGAAGAGGAAAGTAGAATA
AGCAGTTCTCCCACACTTCTGGAGAACAGCCTGGAACAAACTGTGGAGCCAAGTGAAGACCACAGCGAGG
AGGAGAGTGAAGAGGGCAGCGGAGACCTTGGTGAGCCTCTTTATGAAGAGCCATGCAACGAGATAAGCAA
GGAGCAGGCCAAAGCCACCCTTCTGGAGGACCAGCAAGACCCTTCGCCCTCGTCCTAACACCCTGGCTTC
AGTGCAACTCTTTTCCTTCAGCTGCATGTGATTTTGTGATAAAGTTCAGGTAACAGGATGGGCAGTGATG
GAGAATCACTGTTGATTGAGATTTTGGTTTCCATGTGATTTGTTTTCTTCAATAGTCTTATTTTCAGTCT
CTCAAATACAGCCAACTTAAAGTTTTAGTTTGGTGTTTATTGAAAATTAACCAAACTTAATACTAGGAGA
AGACTGAATCATTAATGATGTCTCACAAATTACTGTGTACCTAAGTGGTGTGATGTAAATACTGGAAACA
AAAACAGCAGTTGCATTGATTTTGAAAACAAACCCCCTTGTTATCTGAACATGTTTTCTTCAGGAACAAC
CAGAGGTATCACAAACACTGTTACTCATCTACTGGCTCAGACTGTACTACTTTTTTTTTTTTTTTTCCTG
AAAAAGAAACCAGAAAAAAATGTACTCTTACTGAGATACCCTCTCACCCCAAATGTGTAATGGAAAATTT
TTAATTAAGAAAAACTTCAGTTTTGCCAAGTGCAATGGTGTTGCCTTCTTTAAAAAATGCCGTTTTCTTA
CACTACCAGTGGATGTCCAGACATGCTCTTAGTCTACTAGAGAGGTGCTGCCTTTTCTAAGTCATAATGA
GGAACAGTCCCTTAATTTCTTGTGTGCAACTCTGTTTTATCCTAGAACTAAGAGAGCATTGGTTTGTTAA
AGAGCTTTCAATGTATATTAAAACCTTCAATACTCAGAAATGATGGATTCCTCCAAGGAGTCCTTTACTA
GCCTAAACATTCTCAAATGTTTGAGATTCAAGTGAATGGAAGGAAAACCACATGCCTTTAAAACTAAACT
GTAATAATTACCTGGCTAATTTCAGCTAAGCCTTCATCATAATTTGTTCCCTCAGTAATAGGAGAAATAT
AAATACAGTAAGTTTAGATTATTGAATTGGTGCTTGAAATTTATTGGTTTTGTTGTAATTTTATACAGAT
TATATGAGGGATAAGATACTCATCAAATTGCAAATTCTTTTTTTTACAGAAGTGTGGGTAACAGTCACAG
CAGTTTTTTTTACCAACAGCATACTTAACAGACTTGCTGTGTAGCAGTTTTTTTCTGGTGGAGTTGCTGT
AAGTCTTGTAAGTCTAATGTGGCTATCCTACTCTTTTGGGCAATGCATGTATTATGCATTGGAAAGGTAT
TTTTTTTAAGTTCTGTTGGCTAGCTATGGTTTTCAGTACATTTCCTACTTTAAGAGTAATTACTGACAAA
TATGTATTTCCTATATGTTTATACTTTGATTATAAAAAAGTATTTTGTTTTGATTTTTTAACTTGCTGCA
TTGTTTTGATACTTTCTATTTTTTTGGTCAAATCATGTTTAGAAACTTTGGATGAGTTAAGAAGTCTTAA
GTATGCAGGCGTTTACGTGATTGTGCCATTCCAAAGTGCATCAGAACTGTCATTCCCTTCTAATATCTTC
TCAGGAGTAATACAAATCAGGTATTTCATCATCATTTGGTAATATGAAAACTCCAGTGAACTCCCAAGGA
CATTTACAACATTTATATTCACACGCTGTATGGAAGGGTGTGGGTGTGTGTGAAGGGGCGAGTGGAGACA
CTGTGTGTATCTCTAGATAAGAAGATATGCACCACGTTGAAAATACTCAGTGTAGATCTCTATGTGTATA
GGTATCTGTATATCTTTCCTTTTGTTTACAACTGTTAAAAAACCTCAAAATAGTTCTCTTCAAAAGAAGA
GAGATTCCAAGCAACCCATCTTTCTTCAGTATGTATGTTCTGTACATACTTATCGGAGCGCGCCAGTAAG
TATCAGGCATATATATCTGTCTGTTAGCAATGATTATTACATCATCAGATCAGCATGTGCTATACTCCCT
GCAAGAAATATACTGACATGAACAGGCAGTTCTTGGAGAAGAAAGAGCATTTCTTTAAGTACCTGGGGAA
TACAGCTCTCAGTGATCAGCAGGGAGTTTATTTGAGGACATCAGTCACCTTTGGGGTTGCCATGTACAAT
GAGATTTATAATCATGATACTCTTCGGTGGTAGTTTCAAAAGACACTACTAATACGCAGGAAGCGTTCCA
GCTATTTAATGCTGGCAACTACTGTTTAATGGTCAGTTAAATCTGTGATAATGGTTGGAAGTGGGTGGGG
TTATGAAATTGTAGATGTTTTTAGAAAAACTTGTGAATGAAAATGAATCCAAGTGTTTCATGTGAAGATG
TTGAGCCATTGCTATCATGCATTCCTGTCTCATGGCAGAAAATTTTGAAGATTAAAAAATAAAATAATCA
AAATGTTTCCTCTTTCTAAAAAAAAAAAAAAAAAA

In some embodiments, the disclosed methods and arrays include detecting presence of one or more EZR-ROS, LRIG1-ROS, SLC34A2-ROS, CD74-ROS, SDC4-ROS, and TPM3-ROS gene fusions in a sample from a subject (see, e.g., Rikova et al., Cell 131:1190-1203, 2007). Exemplary nucleic acid sequences of ROS1 fusions detected in at least some embodiments are as follows:

SLC34A2(e4)-ROS(e32) (2175 nt; GenBank Accession No. EU236946)
(SEQ ID NO: 13)
ATGGCTCCCTGGCCTGAATTGGGAGATGCCCAGCCCAACCCCGATAAGTACCTCGAAGGGGCCGCAGGTC
AGCAGCCCACTGCCCCTGATAAAAGCAAAGAGACCAACAAAACAGATAACACTGAGGCACCTGTAACCAA
GATTGAACTTCTGCCGTCCTACTCCACGGCTACACTGATAGATGAGCCCACTGAGGTGGATGACCCCTGG
AACCTACCCACTCTTCAGGACTCGGGGATCAAGTGGTCAGAGAGAGACACCAAAGGGAAGATTCTCTGTT
TCTTCCAAGGGATTGGGAGATTGATTTTACTTCTCGGATTTCTCTACTTTTTCGTGTGCTCCCTGGATAT
TCTTAGTAGCGCCTTCCAGCTGGTTGGAGCTGGAGTCCCAAATAAACCAGGCATTCCCAAATTACTAGAA
GGGAGTAAAAATTCAATACAGTGGGAGAAAGCTGAAGATAATGGATGTAGAATTACATACTATATCCTTG
AGATAAGAAAGAGCACTTCAAATAATTTACAGAACCAGAATTTAAGGTGGAAGATGACATTTAATGGATC
CTGCAGTAGTGTTTGCACATGGAAGTCCAAAAACCTGAAAGGAATATTTCAGTTCAGAGTAGTAGCTGCA
AATAATCTAGGGTTTGGTGAATATAGTGGAATCAGTGAGAATATTATATTAGTTGGAGATGATTTTTGGA
TACCAGAAACAAGTTTCATACTTACTATTATAGTTGGAATATTTCTGGTTGTTACAATCCCACTGACCTT
TGTCTGGCATAGAAGATTAAAGAATCAAAAAAGTGCCAAGGAAGGGGTGACAGTGCTTATAAACGAAGAC
AAAGAGTTGGCTGAGCTGCGAGGTCTGGCAGCCGGAGTAGGCCTGGCTAATGCCTGCTATGCAATACATA
CTCTTCCAACCCAAGAGGAGATTGAAAATCTTCCTGCCTTCCCTCGGGAAAAACTGACTCTGCGTCTCTT
GCTGGGAAGTGGAGCCTTTGGAGAAGTGTATGAAGGAACAGCAGTGGACATCTTAGGAGTTGGAAGTGGA
GAAATCAAAGTAGCAGTGAAGACTTTGAAGAAGGGTTCCACAGACCAGGAGAAGATTGAATTCCTGAAGG
AGGCACATCTGATGAGCAAATTTAATCATCCCAACATTCTGAAGCAGCTTGGAGTTTGTCTGCTGAATGA
ACCCCAATACATTATCCTGGAACTGATGGAGGGAGGAGACCTTCTTACTTATTTGCGTAAAGCCCGGATG
GCAACGTTTTATGGTCCTTTACTCACCTTGGTTGACCTTGTAGACCTGTGTGTAGATATTTCAAAAGGCT
GTGTCTACTTGGAACGGATGCATTTCATTCACAGGGATCTGGCAGCTAGAAATTGCCTTGTTTCCGTGAA
AGACTATACCAGTCCACGGATAGTGAAGATTGGAGACTTTGGACTCGCCAGAGACATCTATAAAAATGAT
TACTATAGAAAGAGAGGGGAAGGCCTGCTCCCAGTTCGGTGGATGGCTCCAGAAAGTTTGATGGATGGAA
TCTTCACTACTCAATCTGATGTATGGTCTTTTGGAATTCTGATTTGGGAGATTTTAACTCTTGGTCATCA
GCCTTATCCAGCTCATTCCAACCTTGATGTGTTAAACTATGTGCAAACAGGAGGGAGACTGGAGCCACCA
AGAAATTGTCCTGATGATCTGTGGAATTTAATGACCCAGTGCTGGGCTCAAGAACCCGACCAAAGACCTA
CTTTTCATAGAATTCAGGACCAACTTCAGTTATTCAGAAATTTTTTCTTAAATAGCATTTATAAGTCCAG
AGATGAAGCAAACAACAGTGGAGTCATAAATGAAAGCTTTGAAGGTGAAGATGGCGATGTGATTTGTTTG
AATTCAGATGACATTATGCCAGTTGCTTTAATGGAAACGAAGAACCGAGAAGGGTTAAACTATATGGTAC
TTGCTACAGAATGTGGCCAAGGTGAAGAAAAGTCTGAGGGTCCTCTAGGCTCCCAGGAATCTGAATCTTG
TGGTCTGAGGAAAGAAGAGAAGGAACCACATGCAGACAAAGATTTCTGCCAAGAAAAACAAGTGGCTTAC
TGCCCTTCTGGCAAGCCTGAAGGCCTGAACTATGCCTGTCTCACTCACAGTGGATATGGAGATGGGTCTG
ATTAA
SLC34A2(E13)-ROS(E32) (1866 nt; GenBank Accession No. EU236947)
(SEQ ID NO: 14)
ATGGCTCCCTGGCCTGAATTGGGAGATGCCCAGCCCAACCCCGATAAGTACCTCGAAGGGGCCGCAGGTA
GCAGCCCACTGCCCCTGATAAAAGCAAAGAGACCAACAAAACAGATAACACTGAGGCACCTGTAACCAAG
ATTGAACTTCTGCCGTCCTACTCCACGGCTACACTGATAGATGAGCCCACTGAGGTGGATGACCCCTGGA
ACCTACCCACTCTTCAGGACTCGGGGATCAAGTGGTCAGAGAGAGACACCAAAGGGAAGATTCTCTGTTT
CTTCCAAGGGATTGGGAGATTGATTTTACTTCTCGGATTTCTCTACTTTTTCGTGTGCTCCCTGGATATT
CTTAGTAGCGCCTTCCAGCTGGTTGGAGATGATTTTTGGATACCAGAAACAAGTTTCATACTTACTATTA
TAGTTGGAATATTTCTGGTTGTTACAATCCCACTGACCTTTGTCTGGCATAGAAGATTAAAGAATCAAAA
AAGTGCCAAGGAAGGGGTGACAGTGCTTATAAACGAAGACAAAGAGTTGGCTGAGCTGCGAGGTCTGGCA
GCCGGAGTAGGCCTGGCTAATGCCTGCTATGCAATACATACTCTTCCAACCCAAGAGGAGATTGAAAATC
TTCCTGCCTTCCCTCGGGAAAAACTGACTCTGCGTCTCTTGCTGGGAAGTGGAGCCTTTGGAGAAGTGTA
TGAAGGAACAGCAGTGGACATCTTAGGAGTTGGAAGTGGAGAAATCAAAGTAGCAGTGAAGACTTTGAAG
AAGGGTTCCACAGACCAGGAGAAGATTGAATTCCTGAAGGAGGCACATCTGATGAGCAAATTTAATCATC
CCAACATTCTGAAGCAGCTTGGAGTTTGTCTGCTGAATGAACCCCAATACATTATCCTGGAACTGATGGA
GGGAGGAGACCTTCTTACTTATTTGCGTAAAGCCCGGATGGCAACGTTTTATGGTCCTTTACTCACCTTG
GTTGACCTTGTAGACCTGTGTGTAGATATTTCAAAAGGCTGTGTCTACTTGGAACGGATGCATTTCATTC
ACAGGGATCTGGCAGCTAGAAATTGCCTTGTTTCCGTGAAAGACTATACCAGTCCACGGATAGTGAAGAT
TGGAGACTTTGGACTCGCCAGAGACATCTATAAAAATGATTACTATAGAAAGAGAGGGGAAGGCCTGCTC
CCAGTTCGGTGGATGGCTCCAGAAAGTTTGATGGATGGAATCTTCACTACTCAATCTGATGTATGGTCTT
TTGGAATTCTGATTTGGGAGATTTTAACTCTTGGTCATCAGCCTTATCCAGCTCATTCCAACCTTGATGT
GTTAAACTATGTGCAAACAGGAGGGAGACTGGAGCCACCAAGAAATTGTCCTGATGATCTGTGGAATTTA
ATGACCCAGTGCTGGGCTCAAGAACCCGACCAAAGACCTACTTTTCATAGAATTCAGGACCAACTTCAGT
TATTCAGAAATTTTTTCTTAAATAGCATTTATAAGTCCAGAGATGAAGCAAACAACAGTGGAGTCATAAA
TGAAAGCTTTGAAGGTGAAGATGGCGATGTGATTTGTTTGAATTCAGATGACATTATGCCAGTTGCTTTA
ATGGAAACGAAGAACCGAGAAGGGTTAAACTATATGGTACTTGCTACAGAATGTGGCCAAGGTGAAGAAA
AGTCTGAGGGTCCTCTAGGCTCCCAGGAATCTGAATCTTGTGGTCTGAGGAAAGAAGAGAAGGAACCACA
TGCAGACAAAGATTTCTGCCAAGAAAAACAAGTGGCTTACTGCCCTTCTGGCAAGCCTGAAGGCCTGAAC
TATGCCTGTCTCACTCACAGTGGATATGGAGATGGGTCTGATTAA
CD74(e6)-ROS(e34) (2112 nt; GenBank Accession No. EU236945)
(SEQ ID NO: 15)
ATGCACAGGAGGAGAAGCAGGAGCTGTCGGGAAGATCAGAAGCCAGTCATGGATGACCAGCGCGACCTTA
TCTCCAACAATGAGCAACTGCCCATGCTGGGCCGGCGCCCTGGGGCCCCGGAGAGCAAGTGCAGCCGCGG
AGCCCTGTACACAGGCTTTTCCATCCTGGTGACTCTGCTCCTCGCTGGCCAGGCCACCACCGCCTACTTC
CTGTACCAGCAGCAGGGCCGGCTGGACAAACTGACAGTCACCTCCCAGAACCTGCAGCTGGAGAACCTGC
GCATGAAGCTTCCCAAGCCTCCCAAGCCTGTGAGCAAGATGCGCATGGCCACCCCGCTGCTGATGCAGGC
GCTGCCCATGGGAGCCCTGCCCCAGGGGCCCATGCAGAATGCCACCAAGTATGGCAACATGACAGAGGAC
CATGTGATGCACCTGCTCCAGAATGCTGACCCCCTGAAGGTGTACCCGCCACTGAAGGGGAGCTTCCCGG
AGAACCTGAGACACCTTAAGAACACCATGGAGACCATAGACTGGAAGGTCTTTGAGAGCTGGATGCACCA
TTGGCTCCTGTTTGAAATGAGCAGGCACTCCTTGGAGCAAAAGCCCACTGACGCTCCACCGAAAGATGAT
TTTTGGATACCAGAAACAAGTTTCATACTTACTATTATAGTTGGAATATTTCTGGTTGTTACAATCCCAC
TGACCTTTGTCTGGCATAGAAGATTAAAGAATCAAAAAAGTGCCAAGGAAGGGGTGACAGTGCTTATAAA
CGAAGACAAAGAGTTGGCTGAGCTGCGAGGTCTGGCAGCCGGAGTAGGCCTGGCTAATGCCTGCTATGCA
ATACATACTCTTCCAACCCAAGAGGAGATTGAAAATCTTCCTGCCTTCCCTCGGGAAAAACTGACTCTGC
GTCTCTTGCTGGGAAGTGGAGCCTTTGGAGAAGTGTATGAAGGAACAGCAGTGGACATCTTAGGAGTTGG
AAGTGGAGAAATCAAAGTAGCAGTGAAGACTTTGAAGAAGGGTTCCACAGACCAGGAGAAGATTGAATTC
CTGAAGGAGGCACATCTGATGAGCAAATTTAATCATCCCAACATTCTGAAGCAGCTTGGAGTTTGTCTGC
TGAATGAACCCCAATACATTATCCTGGAACTGATGGAGGGAGGAGACCTTCTTACTTATTTGCGTAAAGC
CCGGATGGCAACGTTTTATGGTCCTTTACTCACCTTGGTTGACCTTGTAGACCTGTGTGTAGATATTTCA
AAAGGCTGTGTCTACTTGGAACGGATGCATTTCATTCACAGGGATCTGGCAGCTAGAAATTGCCTTGTTT
CCGTGAAAGACTATACCAGTCCACGGATAGTGAAGATTGGAGACTTTGGACTCGCCAGAGACATCTATAA
AAATGATTACTATAGAAAGAGAGGGGAAGGCCTGCTCCCAGTTCGGTGGATGGCTCCAGAAAGTTTGATG
GATGGAATCTTCACTACTCAATCTGATGTATGGTCTTTTGGAATTCTGATTTGGGAGATTTTAACTCTTG
GTCATCAGCCTTATCCAGCTCATTCCAACCTTGATGTGTTAAACTATGTGCAAACAGGAGGGAGACTGGA
GCCACCAAGAAATTGTCCTGATGATCTGTGGAATTTAATGACCCAGTGCTGGGCTCAAGAACCCGACCAA
AGACCTACTTTTCATAGAATTCAGGACCAACTTCAGTTATTCAGAAATTTTTTCTTAAATAGCATTTATA
AGTCCAGAGATGAAGCAAACAACAGTGGAGTCATAAATGAAAGCTTTGAAGGTGAAGATGGCGATGTGAT
TTGTTTGAATTCAGATGACATTATGCCAGTTGCTTTAATGGAAACGAAGAACCGAGAAGGGTTAAACTAT
ATGGTACTTGCTACAGAATGTGGCCAAGGTGAAGAAAAGTCTGAGGGTCCTCTAGGCTCCCAGGAATCTG
AATCTTGTGGTCTGAGGAAAGAAGAGAAGGAACCACATGCAGACAAAGATTTCTGCCAAGAAAAACAAGT
GGCTTACTGCCCTTCTGGCAAGCCTGAAGGCCTGAACTATGCCTGTCTCACTCACAGTGGATATGGAGAT
GGGTCTGATTAA

The disclosed methods and arrays also include detecting wild-type (full-length) ROS1 nucleic acids. An exemplary nucleic acid sequence of ROS1 detected in at least some embodiments is as follows:

ROS1 (7368 nt; GenBank Accession No. NM_002944)
(SEQ ID NO: 16)
CAAGCTTTCAAGCATTCAAAGGTCTAAATGAAAAAGGCTAAGTATTATTTCAAAAGGCAAGTATATCCTA
ATATAGCAAAACAAACAAAGCAAAATCCATCAGCTACTCCTCCAATTGAAGTGATGAAGCCCAAATAATT
CATATAGCAAAATGGAGAAAATTAGACCGGCCATCTAAAAATCTGCCATTGGTGAAGTGATGAAGAACAT
TTACTGTCTTATTCCGAAGCTTGTCAATTTTGCAACTCTTGGCTGCCTATGGATTTCTGTGGTGCAGTGT
ACAGTTTTAAATAGCTGCCTAAAGTCGTGTGTAACTAATCTGGGCCAGCAGCTTGACCTTGGCACACCAC
ATAATCTGAGTGAACCGTGTATCCAAGGATGTCACTTTTGGAACTCTGTAGATCAGAAAAACTGTGCTTT
AAAGTGTCGGGAGTCGTGTGAGGTTGGCTGTAGCAGCGCGGAAGGTGCATATGAAGAGGAAGTACTGGAA
AATGCAGACCTACCAACTGCTCCCTTTGCTTCTTCCATTGGAAGCCACAATATGACATTACGATGGAAAT
CTGCAAACTTCTCTGGAGTAAAATACATCATTCAGTGGAAATATGCACAACTTCTGGGAAGCTGGACTTA
TACTAAGACTGTGTCCAGACCGTCCTATGTGGTCAAGCCCCTGCACCCCTTCACTGAGTACATTTTCCGA
GTGGTTTGGATCTTCACAGCGCAGCTGCAGCTCTACTCCCCTCCAAGTCCCAGTTACAGGACTCATCCTC
ATGGAGTTCCTGAAACTGCACCTTTGATTAGGAATATTGAGAGCTCAAGTCCCGACACTGTGGAAGTCAG
CTGGGATCCACCTCAATTCCCAGGTGGACCTATTTTGGGTTATAACTTAAGGCTGATCAGCAAAAATCAA
AAATTAGATGCAGGGACACAGAGAACCAGTTTCCAGTTTTACTCCACTTTACCAAATACTATCTACAGGT
TTTCTATTGCAGCAGTAAATGAAGTTGGTGAGGGTCCAGAAGCAGAATCTAGTATTACCACTTCATCTTC
AGCAGTTCAACAAGAGGAACAGTGGCTCTTTTTATCCAGAAAAACTTCTCTAAGAAAGAGATCTTTAAAA
CATTTAGTAGATGAAGCACATTGCCTTCGGTTGGATGCTATATACCATAATATTACAGGAATATCTGTTG
ATGTCCACCAGCAAATTGTTTATTTCTCTGAAGGAACTCTCATATGGGCGAAGAAGGCTGCCAACATGTC
TGATGTATCTGACCTGAGAATTTTTTACAGAGGTTCAGGATTAATTTCTTCTATCTCCATAGATTGGCTT
TATCAAAGAATGTATTTCATCATGGATGAACTGGTATGTGTCTGTGATTTAGAGAACTGCTCAAACATCG
AGGAAATTACTCCACCCTCTATTAGTGCACCTCAAAAAATTGTGGCTGATTCATACAATGGGTATGTCTT
TTACCTCCTGAGAGATGGCATTTATAGAGCAGACCTTCCTGTACCATCTGGCCGGTGTGCAGAAGCTGTG
CGTATTGTGGAGAGTTGCACGTTAAAGGACTTTGCAATCAAGCCACAAGCCAAGCGAATCATTTACTTCA
ATGACACTGCCCAAGTCTTCATGTCAACATTTCTGGATGGCTCTGCTTCCCATCTCATCCTACCTCGCAT
CCCCTTTGCTGATGTGAAAAGTTTTGCTTGTGAAAACAATGACTTTCTTGTCACAGATGGCAAGGTCATT
TTCCAACAGGATGCTTTGTCTTTTAATGAATTCATCGTGGGATGTGACCTGAGTCACATAGAAGAATTTG
GGTTTGGTAACTTGGTCATCTTTGGCTCATCCTCCCAGCTGCACCCTCTGCCAGGCCGCCCGCAGGAGCT
TTCGGTGCTGTTTGGCTCTCACCAGGCTCTTGTTCAATGGAAGCCTCCTGCCCTTGCCATAGGAGCCAAT
GTCATCCTGATCAGTGATATTATTGAACTCTTTGAATTAGGCCCTTCTGCCTGGCAGAACTGGACCTATG
AGGTGAAAGTATCCACCCAAGACCCTCCTGAAGTCACTCATATTTTCTTGAACATAAGTGGAACCATGCT
GAATGTACCTGAGCTGCAGAGTGCTATGAAATACAAGGTTTCTGTGAGAGCAAGTTCTCCAAAGAGGCCA
GGCCCCTGGTCAGAGCCCTCAGTGGGTACTACCCTGGTGCCAGCTAGTGAACCACCATTTATCATGGCTG
TGAAAGAAGATGGGCTTTGGAGTAAACCATTAAATAGCTTTGGCCCAGGAGAGTTCTTATCCTCTGATAT
AGGAAATGTGTCAGACATGGATTGGTATAACAACAGCCTCTACTACAGTGACACGAAAGGCGACGTTTTT
GTGTGGCTGCTGAATGGGACGGATATCTCAGAGAATTATCACCTACCCAGCATTGCAGGAGCAGGGGCTT
TAGCTTTTGAGTGGCTGGGTCACTTTCTCTACTGGGCTGGAAAGACATATGTGATACAAAGGCAGTCTGT
GTTGACGGGACACACAGACATTGTTACCCACGTGAAGCTATTGGTGAATGACATGGTGGTGGATTCAGTT
GGTGGATATCTCTACTGGACCACACTCTATTCAGTGGAAAGCACCAGACTAAATGGGGAAAGTTCCCTTG
TACTACAGACACAGCCTTGGTTTTCTGGGAAAAAGGTAATTGCTCTAACTTTAGACCTCAGTGATGGGCT
CCTGTATTGGTTGGTTCAAGACAGTCAATGTATTCACCTGTACACAGCTGTTCTTCGGGGACAGAGCACT
GGGGATACCACCATCACAGAATTTGCAGCCTGGAGTACTTCTGAAATTTCCCAGAATGCACTGATGTACT
ATAGTGGTCGGCTGTTCTGGATCAATGGCTTTAGGATTATCACAACTCAAGAAATAGGTCAGAAAACCAG
TGTCTCTGTTTTGGAACCAGCCAGATTTAATCAGTTCACAATTATTCAGACATCCCTTAAGCCCCTGCCA
GGGAACTTTTCCTTTACCCCTAAGGTTATTCCAGATTCTGTTCAAGAGTCTTCATTTAGGATTGAAGGAA
ATGCTTCAAGTTTTCAAATCCTGTGGAATGGTCCCCCTGCGGTAGACTGGGGTGTAGTTTTCTACAGTGT
AGAATTTAGTGCTCATTCTAAGTTCTTGGCTAGTGAACAACACTCTTTACCTGTATTTACTGTGGAAGGA
CTGGAACCTTATGCCTTATTTAATCTTTCTGTCACTCCTTATACCTACTGGGGAAAGGGCCCCAAAACAT
CTCTGTCACTTCGAGCACCTGAAACAGTTCCATCAGCACCAGAGAACCCCAGAATATTTATATTACCAAG
TGGAAAATGCTGCAACAAGAATGAAGTTGTGGTGGAATTTAGGTGGAACAAACCTAAGCATGAAAATGGG
GTGTTAACAAAATTTGAAATTTTCTACAATATATCCAATCAAAGTATTACAAACAAAACATGTGAAGACT
GGATTGCTGTCAATGTCACTCCCTCAGTGATGTCTTTTCAACTTGAAGGCATGAGTCCCAGATGCTTTAT
TGCCTTCCAGGTTAGGGCCTTTACATCTAAGGGGCCAGGACCATATGCTGACGTTGTAAAGTCTACAACA
TCAGAAATCAACCCATTTCCTCACCTCATAACTCTTCTTGGTAACAAGATAGTTTTTTTAGATATGGATC
AAAATCAAGTTGTGTGGACGTTTTCAGCAGAAAGAGTTATCAGTGCCGTTTGCTACACAGCTGATAATGA
GATGGGATATTATGCTGAAGGGGACTCACTCTTTCTTCTGCACTTGCACAATCGCTCTAGCTCTGAGCTT
TTCCAAGATTCACTGGTTTTTGATATCACAGTTATTACAATTGACTGGATTTCAAGGCACCTCTACTTTG
CACTGAAAGAATCACAAAATGGAATGCAAGTATTTGATGTTGATCTTGAACACAAGGTGAAATATCCCAG
AGAGGTGAAGATTCACAATAGGAATTCAACAATAATTTCTTTTTCTGTATATCCTCTTTTAAGTCGCTTG
TATTGGACAGAAGTTTCCAATTTTGGCTACCAGATGTTCTACTACAGTATTATCAGTCACACCTTGCACC
GAATTCTGCAACCCACAGCTACAAACCAACAAAACAAAAGGAATCAATGTTCTTGTAATGTGACTGAATT
TGAGTTAAGTGGAGCAATGGCTATTGATACCTCTAACCTAGAGAAACCATTGATATACTTTGCCAAAGCA
CAAGAGATCTGGGCAATGGATCTGGAAGGCTGTCAGTGTTGGAGAGTTATCACAGTACCTGCTATGCTCG
CAGGAAAAACCCTTGTTAGCTTAACTGTGGATGGAGATCTTATATACTGGATCATCACAGCAAAGGACAG
CACACAGATTTATCAGGCAAAGAAAGGAAATGGGGCCATCGTTTCCCAGGTGAAGGCCCTAAGGAGTAGG
CATATCTTGGCTTACAGTTCAGTTATGCAGCCTTTTCCAGATAAAGCGTTTCTGTCTCTAGCTTCAGACA
CTGTGGAACCAACTATACTTAATGCCACTAACACTAGCCTCACAATCAGATTACCTCTGGCCAAGACAAA
CCTCACATGGTATGGCATCACCAGCCCTACTCCAACATACCTGGTTTATTATGCAGAAGTTAATGACAGG
AAAAACAGCTCTGACTTGAAATATAGAATTCTGGAATTTCAGGACAGTATAGCTCTTATTGAAGATTTAC
AACCATTTTCAACATACATGATACAGATAGCTGTAAAAAATTATTATTCAGATCCTTTGGAACATTTACC
ACCAGGAAAAGAGATTTGGGGAAAAACTAAAAATGGAGTACCAGAGGCAGTGCAGCTCATTAATACAACT
GTGCGGTCAGACACCAGCCTCATTATATCTTGGAGAGAATCTCACAAGCCAAATGGACCTAAAGAATCAG
TCCGTTATCAGTTGGCAATCTCACACCTGGCCCTAATTCCTGAAACTCCTCTAAGACAAAGTGAATTTCC
AAATGGAAGGCTCACTCTCCTTGTTACTAGACTGTCTGGTGGAAATATTTATGTGTTAAAGGTTCTTGCC
TGCCACTCTGAGGAAATGTGGTGTACAGAGAGTCATCCTGTCACTGTGGAAATGTTTAACACACCAGAGA
AACCTTATTCCTTGGTTCCAGAGAACACTAGTTTGCAATTTAATTGGAAGGCTCCATTGAATGTTAACCT
CATCAGATTTTGGGTTGAGCTACAGAAGTGGAAATACAATGAGTTTTACCATGTTAAAACTTCATGCAGC
CAAGGTCCTGCTTATGTCTGTAATATCACAAATCTACAACCTTATACTTCATATAATGTCAGAGTAGTGG
TGGTTTATAAGACGGGAGAAAATAGCACCTCACTTCCAGAAAGCTTTAAGACAAAAGCTGGAGTCCCAAA
TAAACCAGGCATTCCCAAATTACTAGAAGGGAGTAAAAATTCAATACAGTGGGAGAAAGCTGAAGATAAT
GGATGTAGAATTACATACTATATCCTTGAGATAAGAAAGAGCACTTCAAATAATTTACAGAACCAGAATT
TAAGGTGGAAGATGACATTTAATGGATCCTGCAGTAGTGTTTGCACATGGAAGTCCAAAAACCTGAAAGG
AATATTTCAGTTCAGAGTAGTAGCTGCAAATAATCTAGGGTTTGGTGAATATAGTGGAATCAGTGAGAAT
ATTATATTAGTTGGAGATGATTTTTGGATACCAGAAACAAGTTTCATACTTACTATTATAGTTGGAATAT
TTCTGGTTGTTACAATCCCACTGACCTTTGTCTGGCATAGAAGATTAAAGAATCAAAAAAGTGCCAAGGA
AGGGGTGACAGTGCTTATAAACGAAGACAAAGAGTTGGCTGAGCTGCGAGGTCTGGCAGCCGGAGTAGGC
CTGGCTAATGCCTGCTATGCAATACATACTCTTCCAACCCAAGAGGAGATTGAAAATCTTCCTGCCTTCC
CTCGGGAAAAACTGACTCTGCGTCTCTTGCTGGGAAGTGGAGCCTTTGGAGAAGTGTATGAAGGAACAGC
AGTGGACATCTTAGGAGTTGGAAGTGGAGAAATCAAAGTAGCAGTGAAGACTTTGAAGAAGGGTTCCACA
GACCAGGAGAAGATTGAATTCCTGAAGGAGGCACATCTGATGAGCAAATTTAATCATCCCAACATTCTGA
AGCAGCTTGGAGTTTGTCTGCTGAATGAACCCCAATACATTATCCTGGAACTGATGGAGGGAGGAGACCT
TCTTACTTATTTGCGTAAAGCCCGGATGGCAACGTTTTATGGTCCTTTACTCACCTTGGTTGACCTTGTA
GACCTGTGTGTAGATATTTCAAAAGGCTGTGTCTACTTGGAACGGATGCATTTCATTCACAGGGATCTGG
CAGCTAGAAATTGCCTTGTTTCCGTGAAAGACTATACCAGTCCACGGATAGTGAAGATTGGAGACTTTGG
ACTCGCCAGAGACATCTATAAAAATGATTACTATAGAAAGAGAGGGGAAGGCCTGCTCCCAGTTCGGTGG
ATGGCTCCAGAAAGTTTGATGGATGGAATCTTCACTACTCAATCTGATGTATGGTCTTTTGGAATTCTGA
TTTGGGAGATTTTAACTCTTGGTCATCAGCCTTATCCAGCTCATTCCAACCTTGATGTGTTAAACTATGT
GCAAACAGGAGGGAGACTGGAGCCACCAAGAAATTGTCCTGATGATCTGTGGAATTTAATGACCCAGTGC
TGGGCTCAAGAACCCGACCAAAGACCTACTTTTCATAGAATTCAGGACCAACTTCAGTTATTCAGAAATT
TTTTCTTAAATAGCATTTATAAGTCCAGAGATGAAGCAAACAACAGTGGAGTCATAAATGAAAGCTTTGA
AGGTGAAGATGGCGATGTGATTTGTTTGAATTCAGATGACATTATGCCAGTTGCTTTAATGGAAACGAAG
AACCGAGAAGGGTTAAACTATATGGTACTTGCTACAGAATGTGGCCAAGGTGAAGAAAAGTCTGAGGGTC
CTCTAGGCTCCCAGGAATCTGAATCTTGTGGTCTGAGGAAAGAAGAGAAGGAACCACATGCAGACAAAGA
TTTCTGCCAAGAAAAACAAGTGGCTTACTGCCCTTCTGGCAAGCCTGAAGGCCTGAACTATGCCTGTCTC
ACTCACAGTGGATATGGAGATGGGTCTGATTAATAGCGTTGTTTGGGAAATAGAGAGTTGAGATAAACAC
TCTCATTCAGTAGTTACTGAAAGAAAACTCTGCTAGAATGATAAATGTCATGGTGGTCTATAACTCCAAA
TAAACAATGCAACGTTCC

The disclosed methods utilize fusion probes that are complementary to sequences spanning the fusion point of two genes and also include probes that are complementary to the wild-type (full-length) genes, for example fusion point flanking probes. In some examples, a probe is an oligonucleotide of no more than 100 nucleotides in length, such as about 8 to 100 nucleotides in length (for example, about 15 to 100, 20 to 80, 25 to 75, or 25 to 50, such as about 15, 20, 25, 30, 35, 40, 45, 50, 55, 60, 65, 70, 75, 80, 85, 90, 95, or 100 nucleotides). In one non-limiting example, the probe is about 50 nucleotides in length. Exemplary probes of use in the disclosed methods include those shown in Table 1, or the reverse complement thereof.

TABLE 1
Exemplary ALK and ROS fusion and flanking probes and control probes

| | | SEQ |
|---|---|---|
| | | ID |
| Gene | Probe (5′ -> 3′) | NO: |
| EML4-ALK-v5a | CTTGCAGCTCCTGGTGCTTCCGGCGGTACACTTTACTTGAGACT | 17 |
| | GATTTT | |
| EML4-ALK-v6 | CTGGTGCTTCCGGCGGTACACTATTGAGTAGCGCATCACAGAG | 18 |
| | AGCTGTT | |
| EML4-ALK-v4 | TCAGGGCTCATCCAGCATATCTCTATTTCTCTTTCAGGATTCAG | 19 |
| | ATCATG | |
| EML4-ALK-v3a | CCTGGTGCTTCCGGCGGTACACTTGGTTGATGATGACATCTTTA | 20 |
| | TGCTTG | |
| EML4-ALK-v2 | CTGGTGCTTCCGGCGGTACAAGTACAATATTTCATAGTCTCCCG | 21 |
| | AGTTAG | |
| EZR(e9)-ROS(e34) | TTCTGGTATCCAAAAATCATCCAGCTGCTTCTGATGCTTCTCCTC | 22 |
| | CCGGG | |
| EML4 WT | GCACAGTGATTTCATCTTCTTGTTGCTGAACTCGTGACTCAAGA | 23 |
| | GCTGAC | |
| LRIG1(e16)- | CTTTAATCTTCTATGCCAGACATTGCTCTCAATGTGCCCATTGG | 24 |
| ROS(e35) | CCTGAG | |
| TFG-ALK | CTGGTGCTTCCGGCGGTACACATTTTCAGGAATATTGGTGGAAG | 25 |
| | GTCCTG | |
| KIF5B-ALK | CTGGTGCTTCCGGCGGTACACAATCTGTGCAGAATGCCCTCTTC | 26 |
| | TGGCCA | |
| SLC34A2(e4)- | CCTGGTTTATTTGGGACTCCAGCTCCAACCAGCTGGAAGGCGCT | 27 |
| ROS(e32) | ACTAAG | |
| CD74(e6)- | CCTGGTTTATTTGGGACTCCAGCTGCCAGGACCTCCGTTCTCTC | 28 |
| ROS-(e32) | AAAGAT | |
| EML4-ALK-v1 | CTGGTGCTTCCGGCGGTACACTTTAGGTCCTTTCCCAGGTGTGG | 29 |
| | GCTCTA | |
| SDC4(e2)- | CTGGTTTATTTGGGACTCCAGCCAGATCTCCAGAGCCAGACAGC | 30 |
| ROS(e32) | TCAAAG | |
| TPM3(e8)- | CTTTAATCTTCTATGCCAGACTTCTCCGCCTGAGCCTCAAGAGA | 31 |
| ROS(e35) | CTTGAG | |
| ALK-5′ | CAACTGCACGGAGGCGAGCAGGAGTCTAAATGAAACAGACCTG | 32 |
| | GAAGCTC | |
| ALK-3′ | TATTTCCGTTCCCTCTCCCCTCAAATGGCTCATGTCCACATCAAC | 33 |
| | AAGGC | |
| ROS1-3′ | TTCCCGAGGGAAGGCAGGAAGATTTTCAATCTCCTCTTGGGTTG | 34 |
| | GAAGAG | |
| ROS1-3′-2 | CGTTGCCATCCGGGCTTTACGCAAATAAGTAAGAAGGTCTCCTC | 35 |
| | CCTCCA | |
| CD74(e6)- | GAAACTTGTTTCTGGTATCCAAAAATCATCTTTCGGTGGAGCGT | 36 |
| ROS(e34)-2 | CAGTGG | |
| SLC34A2(e13)- | GAATGCCTGGTTTATTTGGGACTCCAGCCTGAGCCTCTCTGCTA | 37 |
| ROS(e32) | ATGGTT | |
| EML4-ALK-v5b-3 | GCCTCCCTGGATCTCCATATCCTCCCCTGAGCTCTGAACCTTTA | 38 |
| | CTTGAG | |
| EML4-ALK-v3b-3 | AGCTCCTGGTGCTTCCGGCGGTACACTTGGCTGTTTTTTTCGCG | 39 |
| | AGTTGA | |
| DDX5 | GATAAGGGCCCTGCCCTACTTCCTCCAAATCGAGGTGCACCAA | 40 |
| | ACCCTCG | |
| ANT | GTGAGAGCCAGTGATGCAGCTAGATTGTGACCCAGGGCTCATG | 41 |
| | GATAAGC | |
| GAPDH | GACCAGGCGCCCAATACGACCAAATCCGTTGACTCCGACCTTC | 42 |
| | ACCTTCC | |
| FBN1 | GGTCCCACGATGATCCCACTTCCATAAGGACATATCTGGCGGA | 43 |
| | AGGCCTC | |

### V. Arrays

Disclosed herein are arrays that can be used to detect ALK gene fusions (such as one or more of EML4-ALK, TFG-ALK, KIF5B-ALK gene fusions, or combinations of two or more thereof) in a sample, for example for use in diagnosing, prognosing, and/or predicting response of a tumor to an ALK inhibitor, as discussed in Section III, above. In some embodiments, the disclosed arrays can also be used to detect presence of ROS gene fusions (such as one or more of EZR(e9)—ROS(e34), LRIG1(e16)—ROS(e35), SLC34A2(e4)—ROS(e32), SLC34A2(e13)—ROS(e32), CD74(e6)—ROS(e32), CD74(e6)-ROS(e34), SDC4(e2)—ROS(e32), TPM(e8)—ROS(e35), or a combination of two or more thereof).

In some embodiments an array can include a solid surface including oligonucleotides capable of specifically hybridizing to each of EML4-ALK variant 1, EML4-ALK variant 2, EML4-ALK variant 3a, EML4-ALK variant 3b, EML4-ALK variant 4, EML4-ALK variant 5a, EML4-ALK variant 5b, EML4-ALK variant 6, EML4 wild type, ALK wild type, TFG-ALK, KIF5B-ALK, EZR(e9)—ROS(e34), LRIG1(e16)—ROS(e35), SLC34A2(e4)—ROS(e32), SLC34A2(e13)—ROS(e32), CD74(e6)—ROS(e32), CD74(e6)—ROS(e34), SDC4(e2)—ROS(e32), TPM(e8)—ROS(e35), and ROS1 wild type.

In other embodiments, the array can include a solid surface including oligonucleotides capable of specifically hybridizing to each of EML4-ALK variant 1, EML4-ALK variant 2, EML4-ALK variant 3a, EML4-ALK variant 3b, EML4-ALK variant 4, EML4-ALK variant 5a, EML4-ALK variant 5b, EML4-ALK variant 6, EML4 wild type, ALK wild type, TFG-ALK, and KIF5B-ALK.

A. Array Substrates

The solid support of the array can be formed from an organic polymer. Suitable materials for the solid support include, but are not limited to: polypropylene, polyethylene, polybutylene, polyisobutylene, polybutadiene, polyisoprene, polyvinylpyrrolidine, polytetrafluoroethylene, polyvinylidene difluoroide, polyfluoroethylene-propylene, polyethylenevinyl alcohol, polymethylpentene, polycholorotrifluoroethylene, polysulfornes, hydroxylated biaxially oriented polypropylene, aminated biaxially oriented polypropylene, thiolated biaxially oriented polypropylene, ethyleneacrylic acid, thylene methacrylic acid, and blends of copolymers thereof (see U.S. Pat. No. 5,985,567).

In general, suitable characteristics of the material that can be used to form the solid support surface include: being amenable to surface activation such that upon activation, the surface of the support is capable of stably (e.g., covalently, electrostatically, reversibly, irreversibly, or permanently) attaching a biomolecule such as an oligonucleotide thereto; amenability to “in situ” synthesis of biomolecules; being chemically inert such that at the areas on the support not occupied by the oligonucleotides or proteins (such as antibodies) are not amenable to non-specific binding, or when non-specific binding occurs, such materials can be readily removed from the surface without removing the oligonucleotides or proteins (such as antibodies).

In another example, a surface activated organic polymer is used as the solid support surface. One example of a surface activated organic polymer is a polypropylene material aminated via radio frequency plasma discharge. Other reactive groups can also be used, such as carboxylated, hydroxylated, thiolated, or active ester groups.

B. Array Formats

A wide variety of array formats can be employed in accordance with the present disclosure. One example includes a linear array of oligonucleotide bands, generally referred to in the art as a dipstick. Another suitable format includes a two-dimensional pattern of discrete cells (such as 4096 squares in a 64 by 64 array). As is appreciated by those skilled in the art, other array formats including, but not limited to slot (rectangular) and circular arrays are equally suitable for use (see U.S. Pat. No. 5,981,185). In some examples, the array is a multi-well plate (such as a 96-well plate). In one example, the array is formed on a polymer medium, which is a thread, membrane or film. An example of an organic polymer medium is a polypropylene sheet having a thickness on the order of about 1 mil. (0.001 inch) to about 20 mil., although the thickness of the film is not critical and can be varied over a fairly broad range. The array can include biaxially oriented polypropylene (BOPP) films, which in addition to their durability, exhibit low background fluorescence.

The array formats of the present disclosure can be included in a variety of different types of formats. A “format” includes any format to which the solid support can be affixed, such as microtiter plates (e.g., multi-well plates), test tubes, inorganic sheets, dipsticks, and the like. For example, when the solid support is a polypropylene thread, one or more polypropylene threads can be affixed to a plastic dipstick-type device; polypropylene membranes can be affixed to glass slides. The particular format is, in and of itself, unimportant. All that is necessary is that the solid support can be affixed thereto without affecting the functional behavior of the solid support or any biopolymer absorbed thereon, and that the format (such as the dipstick or slide) is stable to any materials into which the device is introduced (such as clinical samples and hybridization solutions).

The arrays of the present disclosure can be prepared by a variety of approaches. In one example, oligonucleotide sequences are synthesized separately and then attached to a solid support (see U.S. Pat. No. 6,013,789). In another example, sequences are synthesized directly onto the support to provide the desired array (see U.S. Pat. No. 5,554,501). Suitable methods for coupling oligonucleotides to a solid support and for directly synthesizing the oligonucleotides onto the support are known to those working in the field; a summary of suitable methods can be found in Matson et al., Anal. Biochem. 217:306-10, 1994. In one example, the oligonucleotides are synthesized onto the support using conventional chemical techniques for preparing oligonucleotides on solid supports (such as PCT applications WO 85/01051 and WO 89/10977, or U.S. Pat. No. 5,554,501).

A suitable array can be produced using automated means to synthesize oligonucleotides in the cells of the array by laying down the precursors for the four bases in a predetermined pattern. Briefly, a multiple-channel automated chemical delivery system is employed to create oligonucleotide probe populations in parallel rows (corresponding in number to the number of channels in the delivery system) across the substrate. Following completion of oligonucleotide synthesis in a first direction, the substrate can then be rotated by 90° to permit synthesis to proceed within a second set of rows that are now perpendicular to the first set. This process creates a multiple-channel array whose intersection generates a plurality of discrete cells.

The oligonucleotides can be bound to the polypropylene support by either the 3′ end of the oligonucleotide or by the 5′ end of the oligonucleotide. In one example, the oligonucleotides are bound to the solid support by the 3′ end. However, one of skill in the art can determine whether the use of the 3′ end or the 5′ end of the oligonucleotide is suitable for bonding to the solid support. In general, the internal complementarity of an oligonucleotide probe in the region of the 3′ end and the 5′ end determines binding to the support.

C. ALK or ROS Gene Fusion Arrays

In some embodiments the array includes or consists essentially of oligonucleotides that include at least a portion that is complementary to one or more of EML4-ALK variant 1, EML4-ALK variant 2, EML4-ALK variant 3a, EML4-ALK variant 3b, EML4-ALK variant 4, EML4-ALK variant 5a, EML4-ALK variant 5b, EML4-ALK variant 6, EML4 wild type, ALK, TFG-ALK, KIF5B-ALK, EZR(e9)-ROS(e34), LRIG1(e16)—ROS(e35), SLC34A2(e4)—ROS(e32), SLC34A2(e13)-ROS(e32), CD74(e6)—ROS(e32), CD74(e6)—ROS(e34), SDC4(e2)—ROS(e32), TPM(e8)-ROS(e35), ROS1, or a combination of two or more thereof. In some examples, the array further includes one or more control oligonucleotides (such as 1, 2, 3, 4, 5, 6, 7, 8, 9, 10, or more control oligonucleotides), for example, one or more positive and/or negative controls. In some examples, the control oligonucleotides are complementary to one or more of DEAD box polypeptide 5 (DDX5), glyceraldehyde-3-phosphate dehydrogenase (GAPDH), fibrillin 1 (FBN1), or Arabidopsis thaliana AP2-like ethylene-responsive transcription factor (ANT).

In some embodiments, the array can include a surface having spatially discrete regions, each region including an anchor stably (e.g., covalently) attached to the surface and a bifunctional linker (“programming linker” or “capture probe”) which has a first portion complementary to the anchor and a second portion complementary to a target nucleic acid. In some examples, an anchor is an oligonucleotide of no more than 500 nucleotides in length, such as about 8 to 500 nucleotides in length (for example, about 10 to 250, 15 to 100, 20 to 80, 25 to 75, or 25 to 50, such as about 15, 20, 25, 30, 35, 40, 45, 50, 55, 60, 65, 70, 75, 80, 85, 90, 95, 100, 110, 120, 130, 140, 150, 200, 225, 250, 275, 300, 325, 350, 375, 400, 425, 450, 475, or 500 nucleotides). In one non-limiting example, the anchor is about 25 nucleotides in length. In some examples, bifunctional linker is an oligonucleotide of no more than 500 nucleotides in length, such as about 8 to 500 nucleotides in length (for example, about 10 to 250, 15 to 100, 20 to 80, 25 to 75, or 25 to 50, such as about 15, 20, 25, 30, 35, 40, 45, 50, 55, 60, 65, 70, 75, 80, 85, 90, 95, 100, 110, 120, 130, 140, 150, 200, 225, 250, 275, 300, 325, 350, 375, 400, 425, 450, 475, or 500 nucleotides). In one non-limiting example, the bifunctional linker is about 50 nucleotides in length. In some examples, the bifunctional linker includes SEQ ID NOs: 44-66.

In some examples, a collection of up to 36 different anchor oligonucleotides can be spotted onto the surface at spatially distinct locations and stably associated with (e.g., covalently attached to) the derivatized surface. For any particular assay, a given set of linkers can be used to program the surface of each well to be specific for as many as 36 different targets or assay types of interest, and different test samples can be applied to each of the 96 wells in each plate. The same set of anchors can be used multiple times to re-program the surface of the wells for other targets and assays of interest.

In other embodiments, the array includes at least two surfaces (for example, 2, 3, 4, 5, 6, 7, 8, 9, 10, 11, 12, 13, 14, 15, 16, 17, 18, 19, 20, 21, 22, 23, 24, 25, or more surfaces), such as a population of beads or other particles or microfluidic channels, wherein each surface (such as each bead or sub-population of beads within a mixed bead population) includes at least one anchor stably attached to the surface and a bifunctional linker including a first portion complementary to the anchor and a second portion complementary to a target nucleic acid. The array can include 2-100 surfaces, such as 2-50 surfaces, 10-100 surfaces, 2-25 surfaces, 5-50 surfaces, or any number of surfaces between 2 and 100. In some examples, the bifunctional linker comprises any one of SEQ ID NOs: 44-66. In some embodiments, each surface included in the array includes substantially similar anchors (for example, the same anchor), which are substantially different from the anchors on the other surfaces in the array. The array can include substantially similar first anchors stably attached to a first surface and substantially similar second anchors attached to a second surface, wherein the first and second anchors are substantially different from each other. The array also includes a first bifunctional linker having a portion complementary to the first anchor and a second portion complementary to a first target nucleic acid (such as any one of SEQ ID NOs: 44-66) and a second bifunctional linker which has a first portion complementary to the second anchor and a second portion complementary to a second target nucleic acid (such as any one of SEQ ID NOs: 44-66). In some embodiments the array may also include substantially similar third anchors attached to a third surface and a third bifunctional linker, substantially similar fourth anchors attached to a fourth surface and a fourth bifunctional linker, and so on, wherein each of the anchors is substantially different from each other.

In some examples, the array includes bifunctional linkers in which the first portion is complementary to an anchor and the second portion is complementary to EML4-ALK variant 1, EML4-ALK variant 2, EML4-ALK variant 3a, EML4-ALK variant 3b, EML4-ALK variant 4, EML4-ALK variant 5a, EML4-ALK variant 5b, EML4-ALK variant 6, EML4 wild type, ALK, TFG-ALK, KIF5B-ALK, EZR(e9)-ROS(e34), LRIG1(e16)—ROS(e35), SLC34A2(e4)—ROS(e32), SLC34A2(e13)-ROS(e32), CD74(e6)—ROS(e32), CD74(e6)—ROS(e34), SDC4(e2)—ROS(e32), TPM(e8)-ROS(e35), or ROS1. In one example, the array includes bifunctional linkers having the nucleic acid sequences of SEQ ID NOs: 44-66 (programming linkers in Table 2) or bifunctional linkers having the nucleic acid sequences of the reverse complement of SEQ ID NOs: 44-66. In another example, the array further includes bifunctional linkers in which the second portion of the bifunctional linker is complementary to a control gene (such as DDX5, GAPDH, FBN1, or ANT). In some examples, the array further includes bifunctional linkers having the nucleic acid sequences of SEQ ID NOs: 67-70 (control gene programming linkers in Table 4) or bifunctional linkers having the nucleic acid sequences of the reverse complement of SEQ ID NOs: 67-70. In one example, the array includes bifunctional linkers consisting of SEQ ID NOs: 44-70 or bifunctional linkers having the nucleic acid sequences of the reverse complement of SEQ ID NOs: 44-70.

TABLE 2
Exemplary ALK and ROS fusion and wild type programming linkers

| | | SEQ |
|---|---|---|
| Programming | | ID |
| Linker | Sequence (5′ -> 3′) | NO: |
| EML4-ALK-v5a | GCAGCGCACGTGCTCAGCCGTAGTGAAAATCAGTCTCAAGTAAAGTGTAC | 44 |
| EML4-ALK-v6 | TGGCTGTAGAACACGCGAGCGGTTCAACAGCTCTCTGTGATGCGCTACTC | 45 |
| EML4-ALK-v4 | CTGGCAGCCACGGACGCGGAACGAGCATGATCTGAATCCTGAAAGAGAAA | 46 |
| EML4-ALK-v3a | CGAAGAGATGCATAACGCGGCGCGCCAAGCATAAAGATGTCATCATCAAC | 47 |
| EML4-ALK-v2 | GGAAGAGCTGGCCGACGGACTGACGCTAACTCGGGAGACTATGAAATATT | 48 |
| EZR(e9)- | GGTACTAGCATGTGGTTAACTGGATCCCGGGAGGAGAAGCATCAGAAGCA | 49 |
| ROS(e34) | | |
| EML4 WT | GGCTATGAACCTCGGCCAACGCTAAGTCAGCTCTTGAGTCACGAGTTCAG | 50 |
| LRIG1(e16)- | AGTTGCCGGGCGTTCCAGACCGAGACTCAGGCCAATGGGCACATTGAGAG | 51 |
| ROS(e35) | | |
| TFG-ALK | GCCACCGACCGAAGACTTACATGATCAGGACCTTCCACCAATATTCCTGA | 52 |
| KIF5B-ALK | GCCACGTAGGCACCGGAGGACTCAGTGGCCAGAAGAGGGCATTCTGCACA | 53 |
| SLC34A2(e4)- | CAAGGACTCTACCGGATCATATGCGCTTAGTAGCGCCTTCCAGCTGGTTG | 54 |
| ROS(e32) | | |
| CD74(e6)- | AACACGTACGGAGCCGGCCCTGTCAATCTTTGAGAGAACGGAGGTCCTGG | 55 |
| ROS-(e32) | | |
| EML4-ALK-v1 | AGGAGCTCCGCGAGGGACATGGTAGTAGAGCCCACACCTGGGAAAGGACC | 56 |
| SDC4(e2)- | ACCTGATAACCACAGTTTCTCCCGCCTTTGAGCTGTCTGGCTCTGGAGAT | 57 |
| ROS(e32) | | |
| TPM3(e8)- | GAACACATACCAGGGCGACAGTCGCCTCAAGTCTCTTGAGGCTCAGGCGG | 58 |
| ROS(e35) | | |
| ALK-5′ | GATGATTTAGGTTGCGCCGCACGAGGAGCTTCCAGGTCTGTTTCATTTAG | 59 |
| ALK-3′ | AAACCCACATAGGGACGCAGCGGATGCCTTGTTGATGTGGACATGAGCCA | 60 |
| ROS1-3′ | CCAGTTGAAGCTATCGCGAAGCCGACTCTTCCAACCCAAGAGGAGATTGA | 61 |
| ROS1-3′-2 | CTTCTTTCACCACGGGCTGGTTCGATGGAGGGAGGAGACCTTCTTACTTA | 62 |
| CD74(e6)- | ACAATGTGGTTCGGAGTGCCGTTCCCCACTGACGCTCCACCGAAAGATGA | 63 |
| ROS(e34) | | |
| SLC34A2(e13)- | TCTGATCTTCCACCGCTCCCGAAAGAACCATTAGCAGAGAGGCTCAGGCT | 64 |
| ROS(e32) | | |
| EML4-ALK-v5b | CAGGGATCAATCTTCCCATACGCGCCTCAAGTAAAGGTTCAGAGCTCAGG | 65 |
| EML4-ALK-v3b | CAGGGTTGCTACGGATTGTGGCAGATCAACTCGCGAAAAAAACAGCCAAG | 66 |

In other embodiments the array includes or consists essentially of oligonucleotides that are complementary to EML4-ALK variant 1, EML4-ALK variant 2, EML4-ALK variant 3a, EML4-ALK variant 3b, EML4-ALK variant 4, EML4-ALK variant 5a, EML4-ALK variant 5b, EML4-ALK variant 6, EML4 wild type, ALK, TFG-ALK, and KIF5B-ALK. In some examples, the array further includes one or more control oligonucleotides (such as 1, 2, 3, 4, 5, 6, 7, 8, 9, 10, or more control oligonucleotides) for example, one or more positive and/or negative controls. In some examples, the control oligonucleotides are complementary to one or more of DEAD box polypeptide 5 (DDX5), glyceraldehyde-3-phosphate dehydrogenase (GAPDH), fibrillin 1 (FBN1), or Arabidopsis thaliana AP2-like ethylene-responsive transcription factor (ANT).

In some embodiments, the array can include a surface including spatially discrete regions, each region including an anchor stably (e.g., covalently) attached to the surface and a bifunctional linker (“programming linker” or “capture probe”) which has a first portion complementary to the anchor and a second portion complementary to a target nucleic acid. In some examples, the array includes bifunctional linkers in which the second portion of the bifunctional linker is complementary to EML4-ALK variant 1, EML4-ALK variant 2, EML4-ALK variant 3a, EML4-ALK variant 3b, EML4-ALK variant 4, EML4-ALK variant 5a, EML4-ALK variant 5b, EML4-ALK variant 6, EML4 wild type, ALK, TFG-ALK, or KIF5B-ALK. In one example, the array includes bifunctional linkers having the nucleic acid sequences of SEQ ID NOs: 44-48, 50, 52-53, 56, 59-60, and 65-66 (programming linkers in Table 3) or bifunctional linkers having the nucleic acid sequences of the reverse complement thereof. In another example, the array further includes bifunctional linkers in which the second portion of the bifunctional linker is complementary to a control gene (such as DDX5, GAPDH, FBN1, or ANT). In some examples, the array further includes bifunctional linkers having the nucleic acid sequences of SEQ ID NOs: 67-70 (control programming linkers in Table 4) or bifunctional linkers having the nucleic acid sequences of the reverse complement of SEQ ID NOs: 67-70. In one example, the array includes bifunctional linkers consisting of SEQ ID NOs: 44-48, 50, 52-53, 56, 59-60, and 65-70 or bifunctional linkers having the nucleic acid sequences of the reverse complement of SEQ ID NOs: 44-48, 50, 52-53, 56, 59-60, and 65-70.

TABLE 3
Exemplary ALK fusion and wild type programming linkers

| | | SEQ |
|---|---|---|
| Programming | | ID |
| Linker | Sequence (5′ -> 3′) | NO: |
| EML4-ALK-v5a | GCAGCGCACGTGCTCAGCCGTAGTGAAAATCAGTCTCAAGTAAAGTGTAC | 44 |
| EML4-ALK-v6 | TGGCTGTAGAACACGCGAGCGGTTCAACAGCTCTCTGTGATGCGCTACTC | 45 |
| EML4-ALK-v4 | CTGGCAGCCACGGACGCGGAACGAGCATGATCTGAATCCTGAAAGAGAAA | 46 |
| EML4-ALK-v3a | CGAAGAGATGCATAACGCGGCGCGCCAAGCATAAAGATGTCATCATCAAC | 47 |
| EML4-ALK-v2 | GGAAGAGCTGGCCGACGGACTGACGCTAACTCGGGAGACTATGAAATATT | 48 |
| EML4 WT | GGCTATGAACCTCGGCCAACGCTAAGTCAGCTCTTGAGTCACGAGTTCAG | 50 |
| TFG-ALK | GCCACCGACCGAAGACTTACATGATCAGGACCTTCCACCAATATTCCTGA | 52 |
| KIF5B-ALK | GCCACGTAGGCACCGGAGGACTCAGTGGCCAGAAGAGGGCATTCTGCACA | 53 |
| EML4-ALK-v1 | AGGAGCTCCGCGAGGGACATGGTAGTAGAGCCCACACCTGGGAAAGGACC | 56 |
| ALK-5′ | GATGATTTAGGTTGCGCCGCACGAGGAGCTTCCAGGTCTGTTTCATTTAG | 59 |
| ALK-3′ | AAACCCACATAGGGACGCAGCGGATGCCTTGTTGATGTGGACATGAGCCA | 60 |
| EML4-ALK-v5b | CAGGGATCAATCTTCCCATACGCGCCTCAAGTAAAGGTTCAGAGCTCAGG | 65 |
| EML4-ALK-v3b | CAGGGTTGCTACGGATTGTGGCAGATCAACTCGCGAAAAAAACAGCCAAG | 66 |

TABLE 4
Exemplary programming linkers for controls

| | | SEQ |
|---|---|---|
| Programming | | ID |
| Linker | Sequence (5′ -> 3′) | NO: |
| DDX5 | GCGGACTGTGGTACCATGCCGACCGCGAGGGTTTGGTGCACCTCGATTTG | 67 |
| ANT | GGACGCCGTCCGGTCCTCACGTGGAGCTTATCCATGAGCCCTGGGTCACA | 68 |
| GAPDH | GCGCTCCCACAACGCTCGACCGGCGGGAAGGTGAAGGTCGGAGTCAACGG | 69 |
| FBN1 | CGTCAGTGAGGAAGAGCGCGATGTGGAGGCCTTCCGCCAGATATGTCCTT | 70 |

### VI. Kits

Also disclosed herein are kits for detecting expression of ALK or ROS1 or gene fusions including ALK or ROS1, or combinations thereof. In some embodiments, the kits include probes for detection of ALK, ROS1, or gene fusions including ALK or ROS1. In one example, the kit includes one or more probes of SEQ ID NOs: 17-39. In some examples, the kit can further include one or more control probes, such as SEQ ID NOs: 40-43. In some embodiments, the kits include an array for detecting expression of ALK, ROS1, or gene fusions including ALK or ROS1, for example one or more arrays disclosed in Section V, above.

In some examples, the kit includes probes for detection of ALK, ROS1, or gene fusions including ALK or ROS1 (for example, one or more of SEQ ID NOs: 17-39) and an array for detecting expression of ALK, ROS1, or gene fusions including ALK or ROS1, such as an array described in Section V, above. The kits may further include additional components, such as one or more programming linkers, such as ALK and/or ROS1 wild type and gene fusion programming linkers described in section V (for example, one or more of SEQ ID NOs: 44-66). The kits may also include control probes (such as SEQ ID NOs: 40-43) and/or control programming linkers (such as SEQ ID NOs: 67-70). In further examples, the kits may also include detection linkers (such as one or more of SEQ ID NOs: 71-93) and/or control detection linkers (such as SEQ ID NOs: 94-97).

The kits may further include additional components such as instructional materials and additional reagents, for example detection reagents, such as an enzyme-based detection system (for example, detection reagents including horseradish peroxidase or alkaline phosphatase and appropriate substrate). The kits may also include additional components to facilitate the particular application for which the kit is designed (for example microtiter plates). Such kits and appropriate contents are well known to those of ordinary skill in the art. The instructional materials may be written, in an electronic form (such as a computer diskette or compact disk) or may be visual (such as video files).

This disclosure also includes methods utilizing integrated systems for high-throughput screening. The systems typically include a robotic armature that transfers fluid from a source to a destination, a controller that controls the robotic armature, a detector, a data storage unit that records detection, and an assay component such as a microtiter plate, for example including one or more programming linkers.

The disclosure is further illustrated by the following non-limiting Examples.

### EXAMPLES

### Example 1

### Detection of ALK Fusions

A quantitative nuclease protection assay is performed in 96-well plates, with a starting sample volume of 30 μl per well in HTG Lysis Buffer containing an appropriate amount of sample to be tested. Probes for all fusions plus controls to be assayed (e.g., the probes shown in Table 1) are included at a starting concentration of 167 μM each, then 70 μl of mineral oil is added per well to prevent evaporation. The plate is heated at 95° C. for 10 minutes, followed by incubation at 60° C. for 6-24 hours. Each well receives 20 μl of S1 enzyme solution and is allowed to incubate at 50° C. for 60-90 minutes. Contents of the plate are transferred to a fresh 96-well plate containing S1 Stop solution and heated at 95° C. for 15-20 minutes to inactivate the enzyme and hydrolyze the protected RNA fragments. Neutralization Solution is added to each well to adjust the pH to about 7.

The contents of each well are transferred into the wells of an ARRAYPLATE which is programmed with the programming linker oligonucleotides shown in Tables 2 and 4. Processed probes are allowed to be captured on the ARRAYPLATE overnight at 60° C. Following washing, detection linker oligonucleotides (e.g., the detection linkers shown in Table 5) are applied and hybridized at 60° C. for 60-90 minutes. The plate is detected using a generic biotinylated detection probe oligonucleotide, and avidin-peroxidase was used to generate a luminescent signal. Plate images are captured on the OMIX HD™, a high resolution CCD imager. The digital images are analyzed with VueScript software that reports the signal intensity of each element on the ARRAYPLATE after correcting for local background. The ratio of signal for the ALK 3′ probe to the ALK 5′ probe is calculated for each sample.

TABLE 5
Detection linkers

| | | SEQ |
|---|---|---|
| Detection | | ID |
| Linker | Sequence (5′ -> 3′) | NO: |
| EML4-ALK-v5a | CGCCGGAAGCACCAGGAGCTGCAAGTGCTCTCCTTCACTGTTTGGAGGTG | 71 |
| EML4-ALK-v6 | AATAGTGTACCGCCGGAAGCACCAGTGCTCTCCTTCACTGTTTGGAGGTG | 72 |
| EML4-ALK-v4 | TAGAGATATGCTGGATGAGCCCTGATGCTCTCCTTCACTGTTTGGAGGTG | 73 |
| EML4-ALK-v3a | CAAGTGTACCGCCGGAAGCACCAGGTGCTCTCCTTCACTGTTTGGAGGTG | 74 |
| EML4-ALK-v2 | GTACTTGTACCGCCGGAAGCACCAGTGCTCTCCTTCACTGTTTGGAGGTG | 75 |
| EZR(e9)- | GCTGGATGATTTTTGGATACCAGAATGCTCTCCTTCACTGTTTGGAGGTG | 76 |
| ROS(e34) | | |
| EML4 WT | CAACAAGAAGATGAAATCACTGTGCTGCTCTCCTTCACTGTTTGGAGGTG | 77 |
| LRIG1(e16)- | CAATGTCTGGCATAGAAGATTAAAGTGCTCTCCTTCACTGTTTGGAGGTG | 78 |
| ROS(e35) | | |
| TFG-ALK | AAATGTGTACCGCCGGAAGCACCAGTGCTCTCCTTCACTGTTTGGAGGTG | 79 |
| KIF5B-ALK | GATTGTGTACCGCCGGAAGCACCAGTGCTCTCCTTCACTGTTTGGAGGTG | 80 |
| SLC34A2(e4)- | GAGCTGGAGTCCCAAATAAACCAGGTGCTCTCCTTCACTGTTTGGAGGTG | 81 |
| ROS(e32) | | |
| CD74(e6)- | CAGCTGGAGTCCCAAATAAACCAGGTGCTCTCCTTCACTGTTTGGAGGTG | 82 |
| ROS-(e32) | | |
| EML4-ALK-v1 | TAAAGTGTACCGCCGGAAGCACCAGTGCTCTCCTTCACTGTTTGGAGGTG | 83 |
| SDC4(e2)- | CTGGCTGGAGTCCCAAATAAACCAGTGCTCTCCTTCACTGTTTGGAGGTG | 84 |
| ROS(e32) | | |
| TPM3(e8)- | AGAAGTCTGGCATAGAAGATTAAAGTGCTCTCCTTCACTGTTTGGAGGTG | 85 |
| ROS(e35) | | |
| ALK-5′ | ACTCCTGCTCGCCTCCGTGCAGTTGTGCTCTCCTTCACTGTTTGGAGGTG | 86 |
| ALK-3′ | TTTGAGGGGAGAGGGAACGGAAATATGCTCTCCTTCACTGTTTGGAGGTG | 87 |
| ROS1-3′ | AAATCTTCCTGCCTTCCCTCGGGAATGCTCTCCTTCACTGTTTGGAGGTG | 88 |
| ROS1-3′-2 | TTTGCGTAAAGCCCGGATGGCAACGTGCTCTCCTTCACTGTTTGGAGGTG | 89 |
| CD74(e6)- | TTTTTGGATACCAGAAACAAGTTTCTGCTCTCCTTCACTGTTTGGAGGTG | 90 |
| ROS(e34) | | |
| SLC34A2(e13)- | GGAGTCCCAAATAAACCAGGCATTCTGCTCTCCTTCACTGTTTGGAGGTG | 91 |
| ROS(e32) | | |
| EML4-ALK-v5b | GGAGGATATGGAGATCCAGGGAGGCTGCTCTCCTTCACTGTTTGGAGGTG | 92 |
| EML4-ALK-v3b | TGTACCGCCGGAAGCACCAGGAGCTTGCTCTCCTTCACTGTTTGGAGGTG | 93 |
| GAPDH | ATTTGGTCGTATTGGGCGCCTGGTCTGCTCTCCTTCACTGTTTGGAGGTG | 94 |
| DDX5 | GAGGAAGTAGGGCAGGGCCCTTATCTGCTCTCCTTCACTGTTTGGAGGTG | 95 |
| FBN1 | ATGGAAGTGGGATCATCGTGGGACCTGCTCTCCTTCACTGTTTGGAGGTG | 96 |
| ANT | ATCTAGCTGCATCACTGGCTCTCACTGCTCTCCTTCACTGTTTGGAGGTG | 97 |

### Example 2

### Predicting Response of a Tumor to ALK Inhibitors

This example describes exemplary methods for predicting response of a tumor to an ALK inhibitor utilizing a quantitative nuclease protection assay and microarray format. However, one skilled in the art will appreciate that methods that deviate from these specific methods can also be used to successfully predict responsiveness of a tumor to an ALK inhibitor.

Lysis buffer (20% formamide, 3×SSC, 0.05% SDS, 1 μg/ml tRNA, and 1 mg/ml Phenol Red), mineral oil (to prevent evaporation), and 167 μM final concentration of one or more fusion probes and/or flanking probes (e.g., the probes shown in Table 1) are added to a sample including tumor cells (such as a fixed or frozen tumor biopsy sample). The sample is heated at 95° C. for 10-15 minutes and then incubated at 60° C. for 6-16 hours for RNA-probe hybridization. If the sample is FFPE tissue or cells, the sample can be treated with 1 mg/ml proteinase K at 50° C. prior to incubation at 60° C. S1 nuclease is diluted 1:40 in S1 nuclease buffer (0.25 M sodium acetate, pH 4.5, 1.4 M NaCl, 0.225 M ZnSO₄, 0.05% KATHON) and added to the sample. The sample is incubated at 50° C. for 60-90 minutes to digest unbound nucleic acids.

An ARRAYPLATE (HTG Molecular) including capture probes at spatially distinct locations (programming linkers) having a portion complementary to a portion of the probe is prepared by diluting 20× wash solution (20×SSC, 0.95% TWEEN-20, 0.05% KATHON), then heated to 50° C. by 1:20, adding 250 μl per well to the ARRAYPLATE, incubated for 10-50 seconds, and emptying the wells. This is repeated for six cycles. After the last wash, 40 μl of programming solution including 5 nM of each capture probe (programming linkers, e.g., Table 2 or Table 3) is added per well and the plate is incubated at 60° C. for 60-90 minutes and then washed.

A Stop plate is prepared with 10 μl S1 stop solution (1.6 N NaOH, 0.135 M EDTA, pH 8.0) and the entire sample (120 μl) is transferred to the stop plate following nuclease incubation. The stop plate is incubated at 95° C. for 15-20 minutes to inactivate the S1 nuclease and hydrolyze bound RNA. The plate is allowed to cool at room temperature for 5-10 minutes and 10 μl neutralization solution (1 M HEPES, pH 7.5, 6×SSC, 1.6 N HCl) is added to the lower aqueous phase of the Stop Plate and mixed.

The wash solution is removed from the ARRAYPLATE and 60 μl of the lower aqueous phase is transferred from the Stop plate to the ARRAYPLATE. The remaining 70 μl of the upper oil phase of the Stop plate is transferred to the ARRAYPLATE and the plate is incubated at 50° C. for 16-24 hours to allow probe hybridization to the plate. The ARRAYPLATE is then washed with wash solution and 40 μl of detection linker solution (5 nM; e.g., Table 5) is added and incubated for 60-90 minutes at 60° C. to allow detection linker hybridization. Following washing, 40 μl of biotinylated detection probe (5 nM) is added to the plate and incubated for 60-90 minutes at 50° C. Following washing, 40 μl of avidin-horseradish peroxidase solution is added to the plate and incubated at 37° C. for 60 minutes. The plate is washed and incubated at room temperature with shaking for 15-30 minutes. After washing, 50 μl of luminescent solution is added and overlaid with 100 μl of imaging oil (99.9% Norpar 15, 0.1% Oil Red 0 Dye) and imaged using an OMIX, OMIX HD, CAPELLA, or SUPERCAPELLA imager. Signal intensity indicates presence and amount of probe hybridization, indicating presence of the target gene fusion (if a fusion probe is used), or presence of full length and/or gene fusion (if flanking probes are used). A ratio of flanking probes (such as a ratio of 3′-ALK probe to 5′-ALK probe) is calculated to determine the presence of an ALK gene fusion in the sample. A subject is identified as having a tumor predicted to respond to an ALK inhibitor such as those disclosed herein if an ALK gene fusion (such as an EML4-ALK, TFG-ALK, or KIF5B-ALK fusion) is present in the sample.

### Example 3

### Determining Prognosis of a Subject

This example describes exemplary methods for determining or predicting prognosis of a subject with a tumor utilizing a quantitative nuclease protection assay and microarray format. However, one skilled in the art will appreciate that methods that deviate from these specific methods can also be used to successfully determine prognosis of a subject with a tumor

Lysis buffer (20% formamide, 3×SSC, 0.05% SDS, 1 μg/ml tRNA, and 1 mg/ml Phenol Red), mineral oil (to prevent evaporation), and 167 μM final concentration of one or more fusion probes and/or flanking probes (e.g., the probes shown in Table 1) are added to a sample including tumor cells (such as a fixed or frozen tumor biopsy sample). The sample is heated at 95° C. for 10-15 minutes and then incubated at 60° C. for 6-16 hours for RNA-probe hybridization. If the sample is FFPE tissue or cells, the sample can be treated with 1 mg/ml proteinase K at 50° C. prior to incubation at 60° C. S1 nuclease is diluted 1:40 in S1 nuclease buffer (0.25 M sodium acetate, pH 4.5, 1.4 M NaCl, 0.225 M ZnSO₄, 0.05% KATHON) and added to the sample. The sample is incubated at 50° C. for 60-90 minutes to digest unbound nucleic acids.

An ARRAYPLATE (HTG Molecular) including capture probes at spatially distinct locations (programming linkers) having a portion complementary to a portion of the probe is prepared by diluting 20× wash solution (20×SSC, 0.95% TWEEN-20, 0.05% KATHON), then heated to 50° C. by 1:20, adding 250 μl per well to the ARRAYPLATE, incubated for 10-50 seconds, and emptying the wells. This is repeated for six cycles. After the last wash, 40 μl of programming solution including 5 nM of each capture probe (programming linkers, e.g., Table 2 or Table 3) is added per well and the plate is incubated at 60° C. for 60-90 minutes and then washed.

A Stop plate is prepared with 10 μl S1 stop solution (1.6 N NaOH, 0.135 M EDTA, pH 8.0) and the entire sample (120 μl) is transferred to the stop plate following nuclease incubation. The stop plate is incubated at 95° C. for 15-20 minutes to inactivate the S1 nuclease and hydrolyze bound RNA. The plate is allowed to cool at room temperature for 5-10 minutes and 10 μl neutralization solution (1 M HEPES, pH 7.5, 6×SSC, 1.6 N HCl) is added to the lower aqueous phase of the Stop Plate and mixed.

The wash solution is removed from the ARRAYPLATE and 60 μl of the lower aqueous phase is transferred from the Stop plate to the ARRAYPLATE. The remaining 70 μl of the upper oil phase of the Stop plate is transferred to the ARRAYPLATE and the plate is incubated at 50° C. for 16-24 hours to allow probe hybridization to the plate. The ARRAYPLATE is then washed with wash solution and 40 μl of detection linker solution (5 nM; e.g., Table 5) is added and incubated for 60-90 minutes at 60° C. to allow detection linker hybridization. Following washing, 40 μl of biotinylated detection probe (5 nM) is added to the plate and incubated for 60-90 minutes at 50° C. Following washing, 40 μl of avidin-horseradish peroxidase solution is added to the plate and incubated at 37° C. for 60 minutes. The plate is washed and incubated at room temperature with shaking for 15-30 minutes. After washing, 50 μl of luminescent solution is added and overlaid with 100 μl of imaging oil (99.9% Norpar 15, 0.1% Oil Red 0 Dye) and imaged using an OMIX, OMIX HD, CAPELLA, or SUPERCAPELLA imager. Signal intensity indicates presence and amount of probe hybridization, indicating presence of the target gene fusion (if a fusion probe is used), or presence of full length and/or gene fusion (if flanking probes are used). A ratio of flanking probes (such as a ratio of 3′-ALK probe to 5′-ALK probe) is calculated to determine the presence of an ALK gene fusion in the sample. A subject is identified as having a poor prognosis if at least one ALK gene fusion (such as an EML4-ALK, TFG-ALK, or KIF5B-ALK fusion) is present in the sample.

### Example 4

### Diagnosis of a Subject

This example describes exemplary methods for diagnosing a subject with a tumor utilizing a quantitative nuclease protection assay and microarray format. However, one skilled in the art will appreciate that methods that deviate from these specific methods can also be used to successfully diagnose a subject with a tumor

Lysis buffer (20% formamide, 3×SSC, 0.05% SDS, 1 μg/ml tRNA, and 1 mg/ml Phenol Red), mineral oil (to prevent evaporation), and 167 μM final concentration of one or more fusion probes and/or flanking probes (e.g., the probes shown in Table 1) are added to a sample including tumor cells (such as a fixed or frozen tumor biopsy sample). The sample is heated at 95° C. for 10-15 minutes and then incubated at 60° C. for 6-16 hours for RNA-probe hybridization. If the sample is FFPE tissue or cells, the sample can be treated with 1 mg/ml proteinase K at 50° C. prior to incubation at 60° C. S1 nuclease is diluted 1:40 in S1 nuclease buffer (0.25 M sodium acetate, pH 4.5, 1.4 M NaCl, 0.225 M ZnSO₄, 0.05% KATHON) and added to the sample. The sample is incubated at 50° C. for 60-90 minutes to digest unbound nucleic acids.

An ARRAYPLATE (HTG Molecular) including capture probes at spatially distinct locations (programming linkers) having a portion complementary to a portion of the probe is prepared by diluting 20× wash solution (20×SSC, 0.95% TWEEN-20, 0.05% KATHON), then heated to 50° C. by 1:20, adding 250 μl per well to the ARRAYPLATE, incubated for 10-50 seconds, and emptying the wells. This is repeated for six cycles. After the last wash, 40 μl of programming solution including 5 nM of each capture probe (programming linkers, e.g., Table 2 or Table 3) is added per well and the plate is incubated at 60° C. for 60-90 minutes and then washed.

A Stop plate is prepared with 10 μl S1 stop solution (1.6 N NaOH, 0.135 M

EDTA, pH 8.0) and the entire sample (120 μl) is transferred to the stop plate following nuclease incubation. The stop plate is incubated at 95° C. for 15-20 minutes to inactivate the S1 nuclease and hydrolyze bound RNA. The plate is allowed to cool at room temperature for 5-10 minutes and 10 μl neutralization solution (1 M HEPES, pH 7.5, 6×SSC, 1.6 N HCl) is added to the lower aqueous phase of the Stop Plate and mixed.

The wash solution is removed from the ARRAYPLATE and 60 μl of the lower aqueous phase is transferred from the Stop plate to the ARRAYPLATE. The remaining 70 μl of the upper oil phase of the Stop plate is transferred to the ARRAYPLATE and the plate is incubated at 50° C. for 16-24 hours to allow probe hybridization to the plate. The ARRAYPLATE is then washed with wash solution and 40 μl of detection linker solution (5 nM; e.g., Table 5) is added and incubated for 60-90 minutes at 60° C. to allow detection linker hybridization. Following washing, 40 μl of biotinylated detection probe (5 nM) is added to the plate and incubated for 60-90 minutes at 50° C. Following washing, 40 μl of avidin-horseradish peroxidase solution is added to the plate and incubated at 37° C. for 60 minutes. The plate is washed and incubated at room temperature with shaking for 15-30 minutes. After washing, 50 μl of luminescent solution is added and overlaid with 100 μl of imaging oil (99.9% Norpar 15, 0.1% Oil Red 0 Dye) and imaged using an OMIX, OMIX HD, CAPELLA, or SUPERCAPELLA imager. Signal intensity indicates presence and amount of probe hybridization, indicating presence of the target gene fusion (if a fusion probe is used), or presence of full length and/or gene fusion (if flanking probes are used). A ratio of flanking probes (such as a ratio of 3′-ALK probe to 5′-ALK probe) is calculated to determine the presence of an ALK gene fusion in the sample. A subject is identified as having a malignant tumor if at least one ALK gene fusion (such as an EML4-ALK, TFG-ALK, or KIF5B-ALK fusion) is present in the sample.

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

We claim:

1. An array comprising a surface comprising spatially discrete regions, each region comprising:
an anchor stably attached to the surface; and
a bifunctional linker which has a first portion complementary to the anchor and a second portion complementary to a target nucleic acid, wherein the bifunctional linker comprises any one of SEQ ID NOs: 44-66.

2. The array of claim of 1, comprising at least two spatially discrete regions, wherein the anchors in each spatially discrete region are (i) substantially the same to each other, and (ii) substantially different from the anchors in other spatially discrete regions.

3. The array of claim 1, wherein the bifunctional linker is no more than 500 base pairs in length.

4. The array of claim 1, wherein the anchor is no more than 500 base pairs in length.

5. The array of claim 1, comprising at least two surfaces, each surface comprising substantially similar anchors, which anchors are substantially different from the anchors on other surfaces.

6. The array of claim 1, comprising at least eight spatially discrete regions, and the bifunctional linkers comprise SEQ ID NO: 44 (EML4-ALK variant 5a), SEQ ID NO: 46 (EML4-ALK variant 4), SEQ ID NO: 47 (EML4-ALK variant 3a), SEQ ID NO: 48 (EML4-ALK variant 2), SEQ ID NO: 50 (EML4 wild type), SEQ ID NO: 56 (EML4-ALK variant 1), SEQ ID NO: 65 (EML4-ALK variant 5b) and SEQ ID NO: 66 (EML4-ALK variant 3b).

7. The array of claim 5 comprising at least eight surfaces, and the bifunctional linkers comprise SEQ ID NO: 44 (EML4-ALK variant 5a), SEQ ID NO: 46 (EML4-ALK variant 4), SEQ ID NO: 47 (EML4-ALK variant 3a), SEQ ID NO: 48 (EML4-ALK variant 2), SEQ ID NO: 50 (EML4 wild type), SEQ ID NO: 56 (EML4-ALK variant 1), SEQ ID NO: 65 (EML4-ALK variant 5b) and SEQ ID NO: 66 (EML4-ALK variant 3b).

8. The array of claim 1, comprising at least 10 spatially discrete regions or surfaces, wherein the target nucleic acid and the bifunctional linker are selected from the group consisting of:
(i) EML4-ALK variant 1, wherein the bifunctional linker comprises SEQ ID NO: 56;
(ii) EML4-ALK variant 2, wherein the bifunctional linker comprises SEQ ID NO: 48;
(iii) EML4-ALK variant 3a, wherein the bifunctional linker comprises SEQ ID NO: 47;
(iv) EML4-ALK variant 3b, wherein the bifunctional linker comprises SEQ ID NO: 66;
(v) EML4-ALK variant 4, wherein the bifunctional linker comprises SEQ ID NO: 46;
(vi) EML4-ALK variant 5a, wherein the bifunctional linker comprises SEQ ID NO: 44;
(vii) EML4-ALK variant 5b, wherein the bifunctional linker comprises SEQ ID NO: 65;
(viii) EML4 wild type, wherein the bifunctional linker comprises SEQ ID NO: 50,
(ix) ALK wild type, wherein the bifunctional linker comprises SEQ ID NO: 59 or 60;
(x) TFG-ALK, wherein the bifunctional linker comprises SEQ ID NO: 52;
(xi) KIF5B-ALK, wherein the bifunctional linker comprises SEQ ID NO: 53;
(xii) EZR(e9)—ROS(e34), wherein the bifunctional linker comprises SEQ ID NO: 49;
(xiii) LRIG1(e16)—ROS(e35), wherein the bifunctional linker comprises SEQ ID NO: 51;
(xiv) SLC34A2(e4)—ROS(e32), wherein the bifunctional linker comprises SEQ ID NO: 54;
(xv) SLC34A2(e13)—ROS(e32), wherein the bifunctional linker comprises SEQ ID NO: 64;
(xvi) CD74(e6)—ROS(e32), wherein the bifunctional linker comprises SEQ ID NO: 55;
(xvii) CD74(e6)—ROS(e34), wherein the bifunctional linker comprises SEQ ID NO: 63;
(xviii) SDC4(e2)—ROS(e32), wherein the bifunctional linker comprises SEQ ID NO: 57;
(xix) TPM(e8)—ROS(e35), wherein the bifunctional linker comprises SEQ ID NO: 58;
(xx) ROS1, wherein the bifunctional linker comprises SEQ ID NO: 61 or 62;
(xxi) EML4-ALK variant 6, wherein the bifunctional linker comprises SEQ ID NO: 45;
and a combination of two or more thereof.

9. An array comprising:
substantially similar first anchors stably attached to a first surface, and substantially similar second anchors attached to a second surface, wherein the first anchors and second anchors are substantially different from each other; and
a first bifunctional linker that has a first portion complementary to the first anchor and a second portion complementary to a first target nucleic acid, wherein the bifunctional linker comprises any one of SEQ ID NOs: 44-66; and
a second bifunctional linker which has a first portion complementary to the second anchor and a second portion complementary to a second target nucleic acid, wherein the bifunctional linker comprises any one of SEQ ID NOs: 44-66, wherein the first target nucleic acid and the second target nucleic acid are substantially different from each other.

10. The array of claim 9, wherein the first surface and second surface are beads or microfluidic channels.

11. The array of claim 1, further comprising at least one bifunctional linker which has a first portion complementary to an anchor and a second portion complementary to a control nucleic acid.

12. The array of claim 11, wherein the control nucleic acid comprises one or more of ANT, GAPDH, DDX5, and FBN1.

13. A method of using the array of claim 1 to detect EML4-ALK variant 1, EML4-ALK variant 2, EML4-ALK variant 3a, EML4-ALK variant 3b, EML4-ALK variant 4, EML4-ALK variant 5a, EML4-ALK variant 5b, EML4 wild type, ALK, TFG-ALK, KIF5B-ALK, EZR(e9)—ROS(e34), LRIG1(e16)—ROS(e35), SLC34A2(e4)—ROS(e32), SLC34A2(e13)—ROS(e32), CD74(e6)—ROS(e32), CD74(e6)—ROS(e34), SDC4(e2)-ROS(e32), TPM(e8)—ROS(e35), and/or ROS1 in a biological sample.

14. A method of detecting a target in a biological sample, comprising contacting the sample with a nucleic acid probe comprising any one of SEQ ID NOs: 17-43, wherein the probe is no more than 100 nucleotides in length, and detecting the specific binding of the probe to a target in the sample.

15. The method of claim 14, wherein the probe consists of any one of SEQ ID NOs: 17-43.

16. A method of predicting response of a tumor in a subject to treatment with a therapeutically effective amount of an anaplastic lymphoma kinase (ALK) inhibitor, comprising:
detecting presence of one or more gene fusions in a sample from the subject using the array of claim 1; and
identifying the tumor as responsive to an ALK inhibitor if EML4-ALK, TFG-ALK, KIF5B-ALK, or a combination of two or more thereof is present in the sample.

17. The method of claim 16, further comprising administering a therapeutically effective amount of an ALK inhibitor to the subject if the tumor is identified as responsive to an ALK inhibitor.

18. The method of claim 16, wherein the ALK inhibitor comprises ASP3026.

19. A method of determining prognosis of a subject with a tumor, comprising:
detecting presence of one or more gene fusions in a sample from the subject using the array of claim 1; and
identifying the subject as having a poor prognosis if one or more gene fusions are present in the sample from the subject.

20. The method of claim 19, wherein the poor prognosis comprises decreased overall survival, decreased relapse-free survival, or decreased metastasis-free survival.

21. A method of determining diagnosis of a subject with a tumor, comprising:
detecting presence of one or more gene fusions in a sample from the subject using the array of claim 1; and
diagnosing the subject as having a malignant tumor if the one or more gene fusions are present in the sample from the subject.

22. The method of claim 16, wherein the tumor comprises a lung tumor, a head and neck tumor, a breast tumor, a gastric tumor, or a lymphoma.

23. The method of claim 16, wherein the sample comprises a tumor biopsy, blood, sputum, or bronchoalveolar lavage.
